SEQUENCE LISTING (704 sequences)

SEQ ID NO: 1 - DNA, 20 bp, Artificial Sequence
  primer
cagcttgtccaactggtcgg

SEQ ID NO: 2 - DNA, 20 bp, artificial
  primer
agcacacaagcacgttaccc

SEQ ID NO: 3 - DNA, 20 bp, artificial
  primer
ggactgagagctgttgacac

SEQ ID NO: 4 - DNA, 17 bp, artificial
  primer
gaccgcgtctcacaccg

SEQ ID NO: 5 - DNA, 26 bp, artificial
  primer
ctgcgctccacgagcccgacgcaatg

SEQ ID NO: 6 - DNA, 26 bp, artificial
  primer
ctggcgtggtcgcgtgctcggcaatg

SEQ ID NO: 7 - DNA, 20 bp, artificial
  primer
gatcagggccgtctcgaaac

SEQ ID NO: 8 - DNA, 20 bp, artificial
  primer
ggactatcatatgcttaccg

SEQ ID NO: 9 - DNA, 20 bp, artificial
  primer
gacccagaattctcgcattc

SEQ ID NO: 10 - DNA, 89 bp, artificial
  primer
aatgatacggcgaccaccgagatctacactatagcctacactctttccctacacgacgctcttccgatcttgtggaaaggacgaaacac

SEQ ID NO: 11 - DNA, 86 bp, artificial
  primer
caagcagaagacggcatacgagatggagatgagtgactggagttcagacgtgtgctcttccgatcttgctaggaccggccttaaag

SEQ ID NO: 12 - DNA, 75 bp, artificial
  primer
aatgatacggcgaccaccgagatctacactctttccctacacgacgctcttccgatctgctggttccatggtgta

SEQ ID NO: 13 - DNA, 88 bp, artificial
  primer
caagcagaagacggcatacgagatcgagtaatgtgactggagttcagacgtgtgctcttccgatctccttagccgctaataggtgagc

SEQ ID NO: 14 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttcacatggatggttactgaagtttgnnngcaggtgcaatggcaacgtcaaatccgtcgacgcattgcacctgcnntatgcaccctcaccttctacccagccgtttgaagagcgtttaaacgctcttcgccctgcccacaaagtatggcggtgtttcgagacggccctga

SEQ ID NO: 15 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcaatacagggatacccactagtttgnnngcaggtgcaatgaccattcgaacctttcggacgcattgcacctgcnntatgcatcatcctgcatgctagaccagtttgaagagcgtttaaacgctcttcgccctgttgagatcgccctttagcgtttcgagacggccctga

SEQ ID NO: 16 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctagatcatacagctgcccaacgtttgnnngcaggtgcaatgccaggcccaaaaagtagcagccattgcacctgcnntatgcaggagagccaacccctaccaggtttgaagagcgtttaaacgctcttcgccctcatcctgcatgctagaccagtttcgagacggccctga

SEQ ID NO: 17 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttgtgatggtgaatattcacagtttgnnngcaggtgcaatgcgcgaggcaacatataaaccccattgcacctgcnntatgcagattccttccaataacacaagtttgaagagcgtttaaacgctcttcgcccctgaaattagacatcaaagcgtttcgagacggccctga

SEQ ID NO: 18 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctaccttgaatgaccagggtacctgcgtttgnnngcaggtgcaatgttccatgacattcccagtgcccattgcacctgcnntatgcatgcccacaaagtatggcggtgtttgaagagcgtttaaacgctcttcgcccagcacacaagcacgttacccgtttcgagacggccctga

SEQ ID NO: 19 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctagcacacaagcacgttacccgtttgnnngcaggtgcaatgctcaacccacgtatgacttcccattgcacctgcnntatgcaagctgtagtagagtccccatgtttgaagagcgtttaaacgctcttcgccccaatacagggatacccactagtttcgagacggccctga

SEQ ID NO: 20 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctacacaacactgatgaggtccgtttgnnngcaggtgcaatgcatacgcgcccattaacttgccattgcacctgcnntatgcagttacactctatctacacctgtttgaagagcgtttaaacgctcttcgccccagtgcaacttactattacggtttcgagacggccctga

SEQ ID NO: 21 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctaccttgcaagccttacctgtagcggtttgnnngcaggtgcaatgctgaatgctgatagacacccccattgcacctgcnntatgcactccacaaagacctgcggcggtttgaagagcgtttaaacgctcttcgccctcatcctgcatgctagaccagtttcgagacggccctga

SEQ ID NO: 22 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctcccaccaggatacgtatgatgtttgnnngcaggtgcaatgcgacccatttccttacccatccattgcacctgcnntatgcactccacaaagacctgcggcggtttgaagagcgtttaaacgctcttcgcccggaagggacactaacctggtgtttcgagacggccctga

SEQ ID NO: 23 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctcgtcaccaagccattgtaccgtttgnnngcaggtgcaatgactcccgcctattcgagttagcattgcacctgcnntatgcactgctagccaacaccactgagtttgaagagcgtttaaacgctcttcgcccttgttacagccttattacacgtttcgagacggccctga

SEQ ID NO: 24 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctactttccatgtcagccatacgtttgnnngcaggtgcaatgtttcccaatcgtccacttcggcattgcacctgcnntatgcacctatcatacgtatcctggtgtttgaagagcgtttaaacgctcttcgcccctgaaattagacatcaaagcgtttcgagacggccctga

SEQ ID NO: 25 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctaccttctgggcatgtgggtccggcgtttgnnngcaggtgcaatgagtctccagtaccccttgaaccattgcacctgcnntatgcatgtgatggtgaatattcacagtttgaagagcgtttaaacgctcttcgcccggagagccaacccctaccaggtttcgagacggccctga

SEQ ID NO: 26 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctacctatcatacgtatcctgggtttgnnngcaggtgcaatgctctatcctgcactgtccagtcattgcacctgcnntatgcatgttgagatcgccctttagcgtttgaagagcgtttaaacgctcttcgccctctgggcatgtgggtccggcgtttcgagacggccctga

SEQ ID NO: 27 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctagatcatacagctgcccaacgtttgnnngcaggtgcaatgcacagtgacgctatcccttaccattgcacctgcnntatgcacaaagacctgcggcgagggcgtttgaagagcgtttaaacgctcttcgcccgccctttagctggtatctgcgtttcgagacggccctga

SEQ ID NO: 28 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctatcagttctgagcaactcgtgtttgnnngcaggtgcaatggaacatagaaccgaccgaccacattgcacctgcnntatgcaacctatcatacgtatcctgggtttgaagagcgtttaaacgctcttcgccctgtgatggtgaatattcacagtttcgagacggccctga

SEQ ID NO: 29 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttcacccataggagccacagcgtttgnnngcaggtgcaatgatgaccaccccaaggaccttccattgcacctgcnntatgcaccctcaccttctacccagccgtttgaagagcgtttaaacgctcttcgccccaagcgcgttagcggtcgtcgtttcgagacggccctga

SEQ ID NO: 30 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctacaggtgacccaaccttcaggtttgnnngcaggtgcaatgaattccgctacgaagccacctcattgcacctgcnntatgcacagcttgtccaactggtcgggtttgaagagcgtttaaacgctcttcgccctcggtgcgctcgctaaaatcgtttcgagacggccctga

SEQ ID NO: 31 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctgtgacttggtgtaagcacaagtttgnnngcaggtgcaatggcaagatccttcaagctaggccattgcacctgcnntatgcacagcttgtccaactggtcgggtttgaagagcgtttaaacgctcttcgcccgagcgatatacgcattgacggtttcgagacggccctga

SEQ ID NO: 32 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctaccttctgggcatgtgggtccggcgtttgnnngcaggtgcaatgatccccgaaacggcaaaacgccattgcacctgcnntatgcattcatagacctaatgtgactgtttgaagagcgtttaaacgctcttcgccccggcgccgcggtcaggtattgtttcgagacggccctga

SEQ ID NO: 33 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctacatagacccctgttgtaaggtttgnnngcaggtgcaatgcgacgagctcaattctatgcccattgcacctgcnntatgcatcacccataggagccacagcgtttgaagagcgtttaaacgctcttcgccccgtatggttcgtggatcgccgtttcgagacggccctga

SEQ ID NO: 34 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctcagtgcaacttactattacggtttgnnngcaggtgcaatgcacaaccgttcaagcgaagctcattgcacctgcnntatgcagaggcggtcaggatggctgcgtttgaagagcgtttaaacgctcttcgccctcgtaccgacgcacattaaggtttcgagacggccctga

SEQ ID NO: 35 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctgattccttccaataacacaagtttgnnngcaggtgcaatgcacaaccacacaggtaggccacattgcacctgcnntatgcaggccatttatagtgttgaccgtttgaagagcgtttaaacgctcttcgcccttccccggcgacactcgcgcgtttcgagacggccctga

SEQ ID NO: 36 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctaccttgtgatggtgaatattcacagtttgnnngcaggtgcaatggacgtaaatgccacctatgcccattgcacctgcnntatgcagttacactctatctacacctgtttgaagagcgtttaaacgctcttcgcccggatacgttcggacgccatcgtttcgagacggccctga

SEQ ID NO: 37 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctctgctagccaacaccactgagtttgnnngcaggtgcaatgttgccaacagaacaacgggctcattgcacctgcnntatgcatcacatggatggttactgaagtttgaagagcgtttaaacgctcttcgcccgcgtcatactatcgttcgtcgtttcgagacggccctga

SEQ ID NO: 38 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctctccacaaagacctgcggcggtttgnnngcaggtgcaatgccacgatacttacacgctccacattgcacctgcnntatgcattcatagacctaatgtgactgtttgaagagcgtttaaacgctcttcgcccctatcaaggtacgtccgttcgtttcgagacggccctga

SEQ ID NO: 39 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctaccttgttgggcagctgtatgatcgtttgnnngcaggtgcaatgccgctttgctttggttctacgcattgcacctgcnntatgcatgcccacaaagtatggcggtgtttgaagagcgtttaaacgctcttcgccctcgcgaccccgatattcacggtttcgagacggccctga

SEQ ID NO: 40 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctctggaaggtgaggcctaggcgtttgnnngcaggtgcaatggccaccagaactgaattgcgacattgcacctgcnntatgcagccctttagctggtatctgcgtttgaagagcgtttaaacgctcttcgccccgtcgttacagtctaccgttgtttcgagacggccctga

SEQ ID NO: 41 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctctggaaggtgaggcctaggcgtttgnnngcaggtgcaatggaatgcaccattggtcaccctcattgcacctgcnntatgcaagaagttggtgacgttctgcgtttgaagagcgtttaaacgctcttcgcccgcgcacaccgcgtcgtaccggtttcgagacggccctga

SEQ ID NO: 42 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctacaggtgacccaaccttcaggtttgnnngcaggtgcaatgcccttaagtcacatcagcccacattgcacctgcnntatgcaccctcaccttctacccagccgtttgaagagcgtttaaacgctcttcgcccggtcggcgcggcatttcgacgtttcgagacggccctga

SEQ ID NO: 43 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctctgcagttgagctggcaatcgtttgnnngcaggtgcaatgaccctgacagttagcgcgtcacattgcacctgcnntatgcactccacaaagacctgcggcggtttgaagagcgtttaaacgctcttcgcccgcgcgacgtaaattcgccttgtttcgagacggccctga

SEQ ID NO: 44 - DNA, 236 bp, artificial
  oligo
  misc_feature (93)..(95): n is a, c, g, or t
  misc_feature (141)..(142): n is a, c, g, or t
gaccgcgtctcacaccgtgttgagatcgccctttagcgtttggagaccgtttaaacggtctctacctccctcaccttctacccagccgtttgnnngcaggtgcaatgaatctaaaaccggcaccgtcccattgcacctgcnntatgcactccacaaagacctgcggcggtttgaagagcgtttaaacgctcttcgccccgcactcgacctacgataccgtttcgagacggccctga

SEQ ID NO: 45 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctgtatcctggtgggatttacagtttgnnngcaggtgcaatgctaaaccacagaatgcgccgacattgcacctgcnntatgcagccctttagctggtatctgcgtttgaagagcgtttaaacgctcttcgcccctagcggcgcaatacgtgctgtttcgagacggccctga

SEQ ID NO: 46 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcaacttcttcgtggtatctcgtttgnnngcaggtgcaatgccaaagtgcaagaatcgcggccattgcacctgcnntatgcaacaggtgacccaaccttcaggtttgaagagcgtttaaacgctcttcgcccgataccgacctctcgccgcggtttcgagacggccctga

SEQ ID NO: 47 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctagcacacaagcacgttacccgtttgnnngcaggtgcaatgcaatttctagcagcgctcccacattgcacctgcnntatgcatgttgagatcgccctttagcgtttgaagagcgtttaaacgctcttcgccccgctcgaccgatatagttacgtttcgagacggccctga

SEQ ID NO: 48 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctagcacacaagcacgttacccgtttgnnngcaggtgcaatgacagtaagaagcaccgcacgtcattgcacctgcnntatgcagccctttagctggtatctgcgtttgaagagcgtttaaacgctcttcgcccctccgcgttagcattaccgcgtttcgagacggccctga

SEQ ID NO: 49 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctcagtgcaacttactattacggtttgnnngcaggtgcaatggtgcactcatttaaggtcccgcattgcacctgcnntatgcagtacatcggacttcgcgtaggtttgaagagcgtttaaacgctcttcgcccgcgatgcaagtacgattcgagtttcgagacggccctga

SEQ ID NO: 50 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcaggcacgatagatacaaaggtttgnnngcaggtgcaatgcattagcagcaacccgtcacccattgcacctgcnntatgcacccctgctcgtcgaggcgatgtttgaagagcgtttaaacgctcttcgcccccgaacctagtaccgcgttagtttcgagacggccctga

SEQ ID NO: 51 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctacatagacccctgttgtaaggtttgnnngcaggtgcaatgcaaaggagccccgactttagtcattgcacctgcnntatgcagtaccgtcaaccgtagcggtgtttgaagagcgtttaaacgctcttcgcccttacacgattaggcgagcacgtttcgagacggccctga

SEQ ID NO: 52 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctgttacactctatctacacctgtttgnnngcaggtgcaatggctcactcacaacgggtttagcattgcacctgcnntatgcagacgtgccattaacgctagggtttgaagagcgtttaaacgctcttcgcccaccgtagtcgtgcatccgtcgtttcgagacggccctga

SEQ ID NO: 53 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttcacatggatggttactgaagtttgnnngcaggtgcaatgttaccgcagcatcgttcactgcattgcacctgcnntatgcactacgcgctaaccgcattgtgtttgaagagcgtttaaacgctcttcgcccgaatttacggcggtcaccgggtttcgagacggccctga

SEQ ID NO: 54 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctctgcagttgagctggcaatcgtttgnnngcaggtgcaatgtacaccctcgaaaggtcaagccattgcacctgcnntatgcaattcgcgttatcgaacacgtgtttgaagagcgtttaaacgctcttcgcccccctacgttacggctccggggtttcgagacggccctga

SEQ ID NO: 55 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctccctcaccttctacccagccgtttgnnngcaggtgcaatggtaactgcggcaccatacttccattgcacctgcnntatgcattgtacgcttcgggcgcgatgtttgaagagcgtttaaacgctcttcgcccctaacggatccccgcgccaggtttcgagacggccctga

SEQ ID NO: 56 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctttgttacagccttattacacgtttgnnngcaggtgcaatggcaggaagatgaagcatcacgcattgcacctgcnntatgcaggtttgcgctaacgtgtggcgtttgaagagcgtttaaacgctcttcgccctagcgaccgtaccgccgattgtttcgagacggccctga

SEQ ID NO: 57 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctaccttctgggcatgtgggtccggcgtttgnnngcaggtgcaatgagcaacttggaactaggcccccattgcacctgcnntatgcatcaggcacggtcgatacgaggtttgaagagcgtttaaacgctcttcgccccgtgattcacgacgatagcggtttcgagacggccctga

SEQ ID NO: 58 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctggactgagagctgttgacacgtttgnnngcaggtgcaatgcagccttaacacgtactagcgcattgcacctgcnntatgcaccggtcatgcggcgaaaccggtttgaagagcgtttaaacgctcttcgccctatctcgcgagcgagttcacgtttcgagacggccctga

SEQ ID NO: 59 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctctggaaggtgaggcctaggcgtttgnnngcaggtgcaatgttccatgcgacaccctcacaacattgcacctgcnntatgcaatttgcgaatccgccacgacgtttgaagagcgtttaaacgctcttcgccctcggagccctattagcgcgagtttcgagacggccctga

SEQ ID NO: 60 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctcccaccaggatacgtatgatgtttgnnngcaggtgcaatgaccaccttccgttactctagccattgcacctgcnntatgcagtggcgtcgagcctatgcgtgtttgaagagcgtttaaacgctcttcgccccgtcgtaataggcattggcggtttcgagacggccctga

SEQ ID NO: 61 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctaatcccttgccccagcacatgtttgnnngcaggtgcaatgatccactaatacttcgcgccccattgcacctgcnntatgcacacctaactacgcccgcgacgtttgaagagcgtttaaacgctcttcgccctctaacgtcggcgctctatagtttcgagacggccctga

SEQ ID NO: 62 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttgtgatggtgaatattcacagtttgnnngcaggtgcaatgaccccgaacaaagaggtcagccattgcacctgcnntatgcagctacggtcgcgcaaaactagtttgaagagcgtttaaacgctcttcgccctgtcatattcgacgccgtacgtttcgagacggccctga

SEQ ID NO: 63 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctatcagttctgagcaactcgtgtttgnnngcaggtgcaatgtccagagccaccggaataatgcattgcacctgcnntatgcacgctacgttacgtccgggttgtttgaagagcgtttaaacgctcttcgccccaacgcgagatcgcatgcaagtttcgagacggccctga

SEQ ID NO: 64 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctggccgagtgcgtaaaaacgagtttgnnngcaggtgcaatggccataagatcctcttccagccattgcacctgcnntatgcagaggaagcttaacgcgcgctgtttgaagagcgtttaaacgctcttcgcccgacagcgcgaacgacctaacgtttcgagacggccctga

SEQ ID NO: 65 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctcggtacggttctccgctacggtttgnnngcaggtgcaatggaccccatagtcccgaattagcattgcacctgcnntatgcaccactcgcggtttataccgggtttgaagagcgtttaaacgctcttcgccctgcgcgatgatctaacgcacgtttcgagacggccctga

SEQ ID NO: 66 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctggttgaccgcgaccgttcgggtttgnnngcaggtgcaatgtcgcgaaagcaagacttagcccattgcacctgcnntatgcacccctcgccatacgcgtaaggtttgaagagcgtttaaacgctcttcgcccccgagtagaccgcgtatggtgtttcgagacggccctga

SEQ ID NO: 67 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctacgtatctcgtccaatcgcagtttgnnngcaggtgcaatgccagacgttagcacactaaggcattgcacctgcnntatgcagttcgaacatgctaccggcggtttgaagagcgtttaaacgctcttcgcccagccgtgccgtattgacgatgtttcgagacggccctga

SEQ ID NO: 68 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctctgtgggttaacgtcgcggtgtttgnnngcaggtgcaatgcccaactattccgcacgttcccattgcacctgcnntatgcaagcaacgaccgcgatcacgtgtttgaagagcgtttaaacgctcttcgcccgcgtcaccggcagactaaacgtttcgagacggccctga

SEQ ID NO: 69 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctgtcacggcggtatactgcgtgtttgnnngcaggtgcaatgtccgtataaagccagctcccacattgcacctgcnntatgcagtacatcgccccggtcgattgtttgaagagcgtttaaacgctcttcgccctcagcgggccgacatcgacggtttcgagacggccctga

SEQ ID NO: 70 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctccaagtaccttctccagcatgtttgnnngcaggtgcaatgcccctaaagggtacttagagccattgcacctgcnntatgcaggggcatgggccatgctgtagtttgaagagcgtttaaacgctcttcgcccgagaggtttccgaagactaagtttcgagacggccctga

SEQ ID NO: 71 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctatccccgtcgcagttgaactgtttgnnngcaggtgcaatggtatactgagcatcccactggcattgcacctgcnntatgcaaaccccaacatcagcaagcggtttgaagagcgtttaaacgctcttcgcccccaagtaccttctccagcatgtttcgagacggccctga

SEQ ID NO: 72 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctaaccccaacatcagcaagcggtttgnnngcaggtgcaatggtgcacgttgcatcacttggacattgcacctgcnntatgcattcctacaaccttctcgcaagtttgaagagcgtttaaacgctcttcgcccgtcacacctgagtaccactggtttcgagacggccctga

SEQ ID NO: 73 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctacctccacagtactcacttggtccgtttgnnngcaggtgcaatgcgaccttcagagtatactcgccattgcacctgcnntatgcaaagcactacccctgccaaccgtttgaagagcgtttaaacgctcttcgcccaaagcagtcgcccctgcttggtttcgagacggccctga

SEQ ID NO: 74 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctgtcacacctgagtaccactggtttgnnngcaggtgcaatgagaatgacgccaactccagagcattgcacctgcnntatgcacagcttcaggaccccgatccgtttgaagagcgtttaaacgctcttcgccctacgctgcaaagtctctggcgtttcgagacggccctga

SEQ ID NO: 75 - DNA, 236 bp, artificial
  oligo
  misc_feature (93)..(95): n is a, c, g, or t
  misc_feature (141)..(142): n is a, c, g, or t
gaccgcgtctcacaccgcagcttcaggaccccgatccgtttggagaccgtttaaacggtctctacctgcaaagccgatgtcagcgccgtttgnnngcaggtgcaatgccactttccacagaacctccgcattgcacctgcnntatgcagtgtctgtatgacccacccggtttgaagagcgtttaaacgctcttcgccctattgcatggcgacaaccgggtttcgagacggccctga

SEQ ID NO: 76 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctaccttaccttgagactccaccctcgtttgnnngcaggtgcaatgccaccatcagataccagcgtacattgcacctgcnntatgcagtgtctgtatgacccacccggtttgaagagcgtttaaacgctcttcgcccctaccgcggtgtagcgcgaagtttcgagacggccctga

SEQ ID NO: 77 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctttcttctcagttcaagtaacgtttgnnngcaggtgcaatgcatcgagcacggcataaagtccattgcacctgcnntatgcataaccatagtacccagttgtgtttgaagagcgtttaaacgctcttcgcccctcgtcgcatagcgggtgaggtttcgagacggccctga

SEQ ID NO: 78 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctaaccccaacatcagcaagcggtttgnnngcaggtgcaatgcggtattcacagacctcacctcattgcacctgcnntatgcaaagcactacccctgccaaccgtttgaagagcgtttaaacgctcttcgcccggcgccgctatcggataagtgtttcgagacggccctga

SEQ ID NO: 79 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgtggaagtctgtgtcgggaagtttgnnngcaggtgcaatgccaaccacatcgttcgattcgcattgcacctgcnntatgcagagaggtttccgaagactaagtttgaagagcgtttaaacgctcttcgcccagtcgttcggcccgtctagtgtttcgagacggccctga

SEQ ID NO: 80 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctggggcatgggccatgctgtagtttgnnngcaggtgcaatgggcagctaattacatagcccccattgcacctgcnntatgcattcctacaaccttctcgcaagtttgaagagcgtttaaacgctcttcgcccacgtagataaccccgcgcgggtttcgagacggccctga

SEQ ID NO: 81 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctccacagtactcacttggtccgtttgnnngcaggtgcaatgtatcaactagcgcacgatcgccattgcacctgcnntatgcataagctgctctctagacggagtttgaagagcgtttaaacgctcttcgcccctcgtatcggaattgggaccgtttcgagacggccctga

SEQ ID NO: 82 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctgtggaagtctgtgtcgggaagtttgnnngcaggtgcaatggatccctatcatcctcggaagcattgcacctgcnntatgcagtagaccaggcacccaacctgtttgaagagcgtttaaacgctcttcgccccggaccgtcgcgtcgtttaagtttcgagacggccctga

SEQ ID NO: 83 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctacctgtcacacctgagtaccactggtttgnnngcaggtgcaatgtaactgcatttaccgcgacgccattgcacctgcnntatgcattcttctcagttcaagtaacgtttgaagagcgtttaaacgctcttcgccctctaccgagggtagcgtccggtttcgagacggccctga

SEQ ID NO: 84 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctacctgagaagcactatgggggcatgtttgnnngcaggtgcaatgtttatgcgcacgatcccagagcattgcacctgcnntatgcatcccacagttgtaccttatcgtttgaagagcgtttaaacgctcttcgccccgtcgttgataggtcgagcggtttcgagacggccctga

SEQ ID NO: 85 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctatcttgcagatgctaatagcgtttgnnngcaggtgcaatgccctattctgaacatcctggccattgcacctgcnntatgcattggattcacgcgtacgagagtttgaagagcgtttaaacgctcttcgcccggtcggttcgctcgtttcgagtttcgagacggccctga

SEQ ID NO: 86 - DNA, 236 bp, artificial
  oligo
  misc_feature (93)..(95): n is a, c, g, or t
  misc_feature (141)..(142): n is a, c, g, or t
gaccgcgtctcacaccggagaggtttccgaagactaagtttggagaccgtttaaacggtctctacctgagccgctgtgatcataccagtttgnnngcaggtgcaatgacgcagccttattaacacggccattgcacctgcnntatgcatgcgcggacgctggtagcgagtttgaagagcgtttaaacgctcttcgccccgattttgggtcgaccgcccgtttcgagacggccctga

SEQ ID NO: 87 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaaccccaacatcagcaagcggtttgnnngcaggtgcaatgctgagccacacgatatgaacgcattgcacctgcnntatgcatcaatgcgccgatcgagatcgtttgaagagcgtttaaacgctcttcgcccacgaccatcgttagcggtgggtttcgagacggccctga

SEQ ID NO: 88 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttaaccatagtacccagttgtgtttgnnngcaggtgcaatgcacccgagaactagccgaaagcattgcacctgcnntatgcagtccccggacattcgacgcggtttgaagagcgtttaaacgctcttcgcccgcgattacgcgtccgtttcggtttcgagacggccctga

SEQ ID NO: 89 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctccacagtactcacttggtccgtttgnnngcaggtgcaatgccgcaaagaatatcacctcgccattgcacctgcnntatgcaagtcgtcgatctctgacgacgtttgaagagcgtttaaacgctcttcgcccgcgcgacacatgggcggttcgtttcgagacggccctga

SEQ ID NO: 90 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctaagcactacccctgccaaccgtttgnnngcaggtgcaatgcgccactgatactcccttaaccattgcacctgcnntatgcatcgattggccactaacggttgtttgaagagcgtttaaacgctcttcgcccacgcgcgttgacgaagtactgtttcgagacggccctga

SEQ ID NO: 91 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctaacatgactccccggaggccgtttgnnngcaggtgcaatggaccccttgatttcacaaccgcattgcacctgcnntatgcaccgttatgtagattggtcgtgtttgaagagcgtttaaacgctcttcgcccacgctacgttatcgccgcgagtttcgagacggccctga

SEQ ID NO: 92 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctttattcttgtccaggcaagggtttgnnngcaggtgcaatgccccaactcgcgaaatgatgacattgcacctgcnntatgcaactcaacgcgcgttggtctagtttgaagagcgtttaaacgctcttcgccccggtcaggccaatttcggcggtttcgagacggccctga

SEQ ID NO: 93 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttgctgtcgtgagtgccagccgtttgnnngcaggtgcaatgccaagactcccagccctaatccattgcacctgcnntatgcataggacgcgcatcgcgtggcgtttgaagagcgtttaaacgctcttcgcccacgatgcgatcggagcgtacgtttcgagacggccctga

SEQ ID NO: 94 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctttcttctcagttcaagtaacgtttgnnngcaggtgcaatgatgtatccagtaaggcccctccattgcacctgcnntatgcacgttcagtcgttagggtacggtttgaagagcgtttaaacgctcttcgccctacgacggaccgattcgatcgtttcgagacggccctga

SEQ ID NO: 95 - DNA, 236 bp, artificial
  oligo
  misc_feature (93)..(95): n is a, c, g, or t
  misc_feature (141)..(142): n is a, c, g, or t
gaccgcgtctcacaccgaaccccaacatcagcaagcggtttggagaccgtttaaacggtctctacctcctgcgatcgctcgtcgaaagtttgnnngcaggtgcaatggactacctcttaccaaggtcccattgcacctgcnntatgcagcccgcagcaatcgtacgcggtttgaagagcgtttaaacgctcttcgcccaccggaataactggcgcgtcgtttcgagacggccctga

SEQ ID NO: 96 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctgtgcaccgtataacgctcgcgtttgnnngcaggtgcaatgtacaccaagtaccaagcggtccattgcacctgcnntatgcacgacgagtttgtctcgataggtttgaagagcgtttaaacgctcttcgccctcccggataagtcggtcgtagtttcgagacggccctga

SEQ ID NO: 97 - DNA, 236 bp, artificial
  oligo
  misc_feature (93)..(95): n is a, c, g, or t
  misc_feature (141)..(142): n is a, c, g, or t
gaccgcgtctcacaccgccacagtactcacttggtccgtttggagaccgtttaaacggtctctacctcgcgcgttgtgtcggctctagtttgnnngcaggtgcaatgcgcaagacctagatcgtcaagcattgcacctgcnntatgcagtcgtggtaccgtttcgtcggtttgaagagcgtttaaacgctcttcgcccacgcgtagtccaggacgaccgtttcgagacggccctga

SEQ ID NO: 98 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctacctactaatccgaactgcgtccggtttgnnngcaggtgcaatgctacgaaaccgagagtatccccattgcacctgcnntatgcacccggacgcgcatcatccatgtttgaagagcgtttaaacgctcttcgccctcgagtccgtaatttaacgtgtttcgagacggccctga

SEQ ID NO: 99 - DNA, 236 bp, artificial
  oligo
  misc_feature (93)..(95): n is a, c, g, or t
  misc_feature (141)..(142): n is a, c, g, or t
gaccgcgtctcacaccgtacgctgcaaagtctctggcgtttggagaccgtttaaacggtctctacctcgtccggtctatcgactaccgtttgnnngcaggtgcaatgctcacagactgtcacttgacgcattgcacctgcnntatgcatgaacacgatacgcgcacgcgtttgaagagcgtttaaacgctcttcgcccataacgccgcgaccaacgtagtttcgagacggccctga

SEQ ID NO: 100 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctggccagaatacaggtcccgcgtttgnnngcaggtgcaatgcagaactcgctcagcacggtacattgcacctgcnntatgcagccttgagcccggtggggctgtttgaagagcgtttaaacgctcttcgccccggccatgcctgccagcttcgtttcgagacggccctga

SEQ ID NO: 101 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgccttgagcccggtggggctgtttgnnngcaggtgcaatgagtgtccaccaaggatctacgcattgcacctgcnntatgcaccgagttacctcgagctttcgtttgaagagcgtttaaacgctcttcgccctgacaacgcggatttggtcggtttcgagacggccctga

SEQ ID NO: 102 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctactcacggcagcgcacgcacgtttgnnngcaggtgcaatgtctcgattgacatcccactcccattgcacctgcnntatgcaaacggcacgtacgccatcgcgtttgaagagcgtttaaacgctcttcgcccccgttccacgacccccgttggtttcgagacggccctga

SEQ ID NO: 103 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctaccttgataagcacctggtgctccgtttgnnngcaggtgcaatgcattcgacattccgctctacgcattgcacctgcnntatgcaaagaggcttcttatgtcggcgtttgaagagcgtttaaacgctcttcgccccgagttgcggcttcggtaacgtttcgagacggccctga

SEQ ID NO: 104 - DNA, 236 bp, artificial
  oligo
  misc_feature (93)..(95): n is a, c, g, or t
  misc_feature (141)..(142): n is a, c, g, or t
gaccgcgtctcacaccgcgcccagggagcgcaaacacgtttggagaccgtttaaacggtctctaccttatccggttacctcatagtcgtttgnnngcaggtgcaatgtccctcttgtaaccgaaagcgcattgcacctgcnntatgcacattgtatacgccttcctgggtttgaagagcgtttaaacgctcttcgccctacgcagccttacgcggtttgtttcgagacggccctga

SEQ ID NO: 105 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctactgtcaggcagcaccactcgtttgnnngcaggtgcaatgatacgtaaccaccgcgaagcccattgcacctgcnntatgcagatcccgacgtatagcccgcgtttgaagagcgtttaaacgctcttcgcccgcggatcgcaatataacgaagtttcgagacggccctga

SEQ ID NO: 106 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctaccttcagaagcatgataagcaccgtttgnnngcaggtgcaatgagcacaactaaccgattggcccattgcacctgcnntatgcacatgagagcgacgatgcgtagtttgaagagcgtttaaacgctcttcgcccaccgccgaatcgagcctgcggtttcgagacggccctga

SEQ ID NO: 107 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctgcatccacattcttacaggcgtttgnnngcaggtgcaatggccccaatcacattcgttcaccattgcacctgcnntatgcagatatcgacggccgattgcggtttgaagagcgtttaaacgctcttcgcccatagggcgctcgattacgcagtttcgagacggccctga

SEQ ID NO: 108 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctttctcggaagtcgaggacaggtttgnnngcaggtgcaatgtcccgacgcaatatatcccggcattgcacctgcnntatgcataacggctactgccgcgcgtgtttgaagagcgtttaaacgctcttcgccctacgttaacgctatgcggcggtttcgagacggccctga

SEQ ID NO: 109 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctgcgccagtgtttgcgctcccgtttgnnngcaggtgcaatgcccaccagttgataaacctcccattgcacctgcnntatgcacgttggggcacgtcgtcgaagtttgaagagcgtttaaacgctcttcgccccgtggcgaatcgcggctaaagtttcgagacggccctga

SEQ ID NO: 110 - DNA, 236 bp, artificial
  oligo
  misc_feature (93)..(95): n is a, c, g, or t
  misc_feature (141)..(142): n is a, c, g, or t
gaccgcgtctcacaccgtacttgtacctgaaagctcggtttggagaccgtttaaacggtctctacctcattgtatacgccttcctgggtttgnnngcaggtgcaatgaccgacattagtcacatgcgccattgcacctgcnntatgcaggcgggcgattgtcgtcgtagtttgaagagcgtttaaacgctcttcgcccgtacaattagtcgttaacgtgtttcgagacggccctga

SEQ ID NO: 111 - DNA, 236 bp, artificial
  oligo
  misc_feature (93)..(95): n is a, c, g, or t
  misc_feature (141)..(142): n is a, c, g, or t
gaccgcgtctcacaccgtcgggggacggttatttctggtttggagaccgtttaaacggtctctacctgttgtgctatatctacgcgtgtttgnnngcaggtgcaatggtcaacatccggcaattccgtcattgcacctgcnntatgcagtggacttcgccgatccgtcgtttgaagagcgtttaaacgctcttcgccctcggacacccgagattgccggtttcgagacggccctga

SEQ ID NO: 112 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgggcggcgtatcgcgtactcgtttgnnngcaggtgcaatgacgaacctagccgtacgcaaacattgcacctgcnntatgcatcctacaaaggcgcgactccgtttgaagagcgtttaaacgctcttcgcccaacgctttaccgggtggaacgtttcgagacggccctga

SEQ ID NO: 113 - DNA, 236 bp, artificial
  oligo
  misc_feature (93)..(95): n is a, c, g, or t
  misc_feature (141)..(142): n is a, c, g, or t
gaccgcgtctcacaccgtgataagcacctggtgctccgtttggagaccgtttaaacggtctctacctgctatacgtcgcgctttaatgtttgnnngcaggtgcaatgcatcgcaggctaatcgctatgcattgcacctgcnntatgcagtataactagtcgcgtgcaagtttgaagagcgtttaaacgctcttcgcccgcttcgttgtcgacaagtcggtttcgagacggccctga

SEQ ID NO: 114 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctaacaaggcgccttagtcgtggtttgnnngcaggtgcaatgcctgataactaactagcgccccattgcacctgcnntatgcatcgagcgtaccgaggatactgtttgaagagcgtttaaacgctcttcgccccgcgttagcgtacgatagtagtttcgagacggccctga

SEQ ID NO: 115 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctagatcatacagctgcccaacgtttgnnngcaggtgcaatgcacgtacgaaccgagatgatgcattgcacctgcnntatgcaacatagacccctgttgtaaggtttgaagagcgtttaaacgctcttcgccctcacatggatggttactgaagtttcgagacggccctga

SEQ ID NO: 116 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctcaggcacgatagatacaaaggtttgnnngcaggtgcaatgcttcctgaacaccgagcacagcattgcacctgcnntatgcacaatacagggatacccactagtttgaagagcgtttaaacgctcttcgcccttgggactcctcggttatttgtttcgagacggccctga

SEQ ID NO: 117 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttgtcgatggcaatagccaaggtttgnnngcaggtgcaatgacagttcaatcagcacgtccccattgcacctgcnntatgcagctcaaaccattacagaagggtttgaagagcgtttaaacgctcttcgcccgtgacttggtgtaagcacaagtttcgagacggccctga

SEQ ID NO: 118 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctttgggactcctcggttatttgtttgnnngcaggtgcaatgtgggcaaccagtcgactatgccattgcacctgcnntatgcagttacactctatctacacctgtttgaagagcgtttaaacgctcttcgccctgcccacaaagtatggcggtgtttcgagacggccctga

SEQ ID NO: 119 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctagatcatacagctgcccaacgtttgnnngcaggtgcaatgcgagaatccctgattacacgccattgcacctgcnntatgcattcatagacctaatgtgactgtttgaagagcgtttaaacgctcttcgccccaacttcttcgtggtatctcgtttcgagacggccctga

SEQ ID NO: 120 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctaccttgttgagatcgccctttagcgtttgnnngcaggtgcaatgtcgcacgaaatggaccgagaacattgcacctgcnntatgcacagtgcaacttactattacggtttgaagagcgtttaaacgctcttcgccccaacttcttcgtggtatctcgtttcgagacggccctga

SEQ ID NO: 121 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctcagtgcaacttactattacggtttgnnngcaggtgcaatggcattctcaacttagctcccgcattgcacctgcnntatgcactgaaattagacatcaaagcgtttgaagagcgtttaaacgctcttcgccccccaccaggatacgtatgatgtttcgagacggccctga

SEQ ID NO: 122 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctcaggcacgatagatacaaaggtttgnnngcaggtgcaatgcactttagccaacgtgtccaccattgcacctgcnntatgcaggactgagagctgttgacacgtttgaagagcgtttaaacgctcttcgccctctttgaaccattgtgttatgtttcgagacggccctga

SEQ ID NO: 123 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctgaggcggtcaggatggctgcgtttgnnngcaggtgcaatgaatgccatagcgcacacagcgcattgcacctgcnntatgcagtgacttggtgtaagcacaagtttgaagagcgtttaaacgctcttcgcccagaagttggtgacgttctgcgtttcgagacggccctga

SEQ ID NO: 124 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttgggactcctcggttatttagtttgnnngcaggtgcaatgcgctttatactcaccacggagcattgcacctgcnntatgcacatcggcctgacccccatgcgtttgaagagcgtttaaacgctcttcgcccattcttctctgtgaatgtcggtttcgagacggccctga

SEQ ID NO: 125 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctggactgagagctgttgacacgtttgnnngcaggtgcaatgcccagcgaagtataacctgagcattgcacctgcnntatgcacccaccaggatacgtatgatgtttgaagagcgtttaaacgctcttcgcccctaaagggcgatctcaacaagtttcgagacggccctga

SEQ ID NO: 126 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctcctatcatacgtatcctggtgtttgnnngcaggtgcaatgtttccggaaacctatcgtcgccattgcacctgcnntatgcaacatagacccctgttgtaaggtttgaagagcgtttaaacgctcttcgcccccctcaccttctacccagccgtttcgagacggccctga

SEQ ID NO: 127 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctaccttcacccataggagccacagcgtttgnnngcaggtgcaatgtgtatatgtccagtcgggcggcattgcacctgcnntatgcaagatcatacagctgcccaacgtttgaagagcgtttaaacgctcttcgcccagctgtagtagagtccccatgtttcgagacggccctga

SEQ ID NO: 128 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctctgctagccaacaccactgagtttgnnngcaggtgcaatgccagtcctaactagcccgtttcattgcacctgcnntatgcacaacttcttcgtggtatctcgtttgaagagcgtttaaacgctcttcgccctgttgggcagctgtatgatcgtttcgagacggccctga

SEQ ID NO: 129 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctacctatcatacgtatcctgggtttgnnngcaggtgcaatgctcttacacccatgcactgcacattgcacctgcnntatgcagccctttagctggtatctgcgtttgaagagcgtttaaacgctcttcgcccagaagttggtgacgttctgcgtttcgagacggccctga

SEQ ID NO: 130 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctctgcagttgagctggcaatcgtttgnnngcaggtgcaatgcccgcctactaaggaacgaatcattgcacctgcnntatgcaacctatcatacgtatcctgggtttgaagagcgtttaaacgctcttcgcccggtcatcgatcgcctctagtgtttcgagacggccctga

SEQ ID NO: 131 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctggaagggacactaacctggtgtttgnnngcaggtgcaatgcgaccgacaccttaacccatgcattgcacctgcnntatgcatgttgggcagctgtatgatcgtttgaagagcgtttaaacgctcttcgccccctacgcagcgacgagtcttgtttcgagacggccctga

SEQ ID NO: 132 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctggaagggacactaacctggtgtttgnnngcaggtgcaatggcctaccaccgaaattcttcccattgcacctgcnntatgcaccctcaccttctacccagccgtttgaagagcgtttaaacgctcttcgcccgtcgcttacgaacgcttacggtttcgagacggccctga

SEQ ID NO: 133 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctcaaagacctgcggcgagggcgtttgnnngcaggtgcaatgcttggtcaaatctcgacgacccattgcacctgcnntatgcatgaatgaccagggtacctgcgtttgaagagcgtttaaacgctcttcgccctaggcgcccattcgacgacggtttcgagacggccctga

SEQ ID NO: 134 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctttcatagacctaatgtgactgtttgnnngcaggtgcaatggtgcaaacagtaaccgactcccattgcacctgcnntatgcactgctagccaacaccactgagtttgaagagcgtttaaacgctcttcgccccgtcggtcacgagtacatgcgtttcgagacggccctga

SEQ ID NO: 135 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctacctatcatacgtatcctgggtttgnnngcaggtgcaatgctactgtcaaccccggttaaccattgcacctgcnntatgcagattccttccaataacacaagtttgaagagcgtttaaacgctcttcgcccggttcgacagcgttaggcccgtttcgagacggccctga

SEQ ID NO: 136 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctgtgacttggtgtaagcacaagtttgnnngcaggtgcaatgcgtccgttaatccgactcactcattgcacctgcnntatgcacatcggcctgacccccatgcgtttgaagagcgtttaaacgctcttcgcccgcccgtatgcgagaatcgaggtttcgagacggccctga

SEQ ID NO: 137 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctaccttgcccacaaagtatggcggtgtttgnnngcaggtgcaatgcactttcactaccacagggaccattgcacctgcnntatgcattgttacagccttattacacgtttgaagagcgtttaaacgctcttcgcccagctcgggtaatgtccgttagtttcgagacggccctga

SEQ ID NO: 138 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctattcttctctgtgaatgtcggtttgnnngcaggtgcaatgccaacgcataaccgctatacccattgcacctgcnntatgcattgggactcctcggttatttgtttgaagagcgtttaaacgctcttcgccccgcggacgatatatcggctcgtttcgagacggccctga

SEQ ID NO: 139 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctttgttacagccttattacacgtttgnnngcaggtgcaatgccaacagacatgccccacagtcattgcacctgcnntatgcacagtgcaacttactattacggtttgaagagcgtttaaacgctcttcgccccggacgaatgaccaaccggggtttcgagacggccctga

SEQ ID NO: 140 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctggccatttatagtgttgaccgtttgnnngcaggtgcaatgagccaaaagatcgccgactctcattgcacctgcnntatgcacaatacagggatacccactagtttgaagagcgtttaaacgctcttcgccctgtcgtgggcagtagtatcggtttcgagacggccctga

SEQ ID NO: 141 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctgctcaaaccattacagaagggtttgnnngcaggtgcaatgcagcacagagagtacaaggcccattgcacctgcnntatgcaacctatcatacgtatcctgggtttgaagagcgtttaaacgctcttcgccccgcacgccgatatcattcgggtttcgagacggccctga

SEQ ID NO: 142 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctcaatacagggatacccactagtttgnnngcaggtgcaatgcacaaaaggaacgcacgactgcattgcacctgcnntatgcatcacccataggagccacagcgtttgaagagcgtttaaacgctcttcgccccgactaccccgggtgcgacagtttcgagacggccctga

SEQ ID NO: 143 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgttacactctatctacacctgtttgnnngcaggtgcaatgctaatcgcgaccacgaagcatcattgcacctgcnntatgcatgaatgaccagggtacctgcgtttgaagagcgtttaaacgctcttcgccctagttcgttcgacggagcgtgtttcgagacggccctga

SEQ ID NO: 144 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctctgcagttgagctggcaatcgtttgnnngcaggtgcaatgtcgcgcaacctcgtcaaatcccattgcacctgcnntatgcacaacttcttcgtggtatctcgtttgaagagcgtttaaacgctcttcgcccattttagtacccgcgccgacgtttcgagacggccctga

SEQ ID NO: 145 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgttgagatcgccctttagcgtttgnnngcaggtgcaatgacgttcttacaccagcgcatgcattgcacctgcnntatgcacgtcaccaagccattgtaccgtttgaagagcgtttaaacgctcttcgcccacccgctccgacgacccaaggtttcgagacggccctga

SEQ ID NO: 146 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctaccttgttgagatcgccctttagcgtttgnnngcaggtgcaatggctaacttgcacgaatcgagccattgcacctgcnntatgcaattcttctctgtgaatgtcggtttgaagagcgtttaaacgctcttcgcccaacgatccggtaggtcggccgtttcgagacggccctga

SEQ ID NO: 147 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctgtatcctggtgggatttacagtttgnnngcaggtgcaatgcgaagattgcacacggtaagccattgcacctgcnntatgcaagcacacaagcacgttacccgtttgaagagcgtttaaacgctcttcgcccatctgtattaacgcggtacagtttcgagacggccctga

SEQ ID NO: 148 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctcgtcaccaagccattgtaccgtttgnnngcaggtgcaatgcgactcccttactcagacgaccattgcacctgcnntatgcagccctttagctggtatctgcgtttgaagagcgtttaaacgctcttcgccctcccgccgcgtacctatgtagtttcgagacggccctga

SEQ ID NO: 149 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctattcttctctgtgaatgtcggtttgnnngcaggtgcaatggacctattgataaccccagcgcattgcacctgcnntatgcagaggcggtcaggatggctgcgtttgaagagcgtttaaacgctcttcgcccagattctcatcgcgcggattgtttcgagacggccctga

SEQ ID NO: 150 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctagatcatacagctgcccaacgtttgnnngcaggtgcaatggcacgaccaacatcctcgaatcattgcacctgcnntatgcaagttgaacgcgtcaccgcgtgtttgaagagcgtttaaacgctcttcgccctagacctcgacgaacgctttgtttcgagacggccctga

SEQ ID NO: 151 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttctttgaaccattgtgttatgtttgnnngcaggtgcaatggtccaacatgaacccgcttagcattgcacctgcnntatgcagcgagcttaccgactttccggtttgaagagcgtttaaacgctcttcgcccgcgagcgcggaaaattcggagtttcgagacggccctga

SEQ ID NO: 152 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctgattccttccaataacacaagtttgnnngcaggtgcaatgaccgacgttacatagacgacgcattgcacctgcnntatgcaaggggcgacatcgttttacggtttgaagagcgtttaaacgctcttcgccccgggctattataacgacggggtttcgagacggccctga

SEQ ID NO: 153 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctaatcccttgccccagcacatgtttgnnngcaggtgcaatgtcgtaacatacgcgcaatccccattgcacctgcnntatgcacgggcaacgaacgcgggcacgtttgaagagcgtttaaacgctcttcgccctagcatgcgctttgcggcgagtttcgagacggccctga

SEQ ID NO: 154 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctggaagggacactaacctggtgtttgnnngcaggtgcaatgcttcgaaaagccccagagctgcattgcacctgcnntatgcatacatgcacagcgcgcgacggtttgaagagcgtttaaacgctcttcgcccaattcgcgcgaattggcggggtttcgagacggccctga

SEQ ID NO: 155 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctaccttctgggcatgtgggtccggcgtttgnnngcaggtgcaatgcgatacagcaacgtccatgaccattgcacctgcnntatgcacgataggtacccgtagcgaagtttgaagagcgtttaaacgctcttcgcccttcccgcgcctactatcggcgtttcgagacggccctga

SEQ ID NO: 156 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctacacaacactgatgaggtccgtttgnnngcaggtgcaatgcagcagaggctacaattaccgcattgcacctgcnntatgcatctcgggcgacgccgaaatagtttgaagagcgtttaaacgctcttcgcccatctacgtaccgttagcggagtttcgagacggccctga

SEQ ID NO: 157 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctgttacactctatctacacctgtttgnnngcaggtgcaatgctacgtagaccaatgcaccaccattgcacctgcnntatgcagcgcggttccgcggatattcgtttgaagagcgtttaaacgctcttcgcccttacgcagcgacgtcttaatgtttcgagacggccctga

SEQ ID NO: 158 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctaccttctgggcatgtgggtccggcgtttgnnngcaggtgcaatgtcaatgaccggcacggaaacccattgcacctgcnntatgcacatcgggttaaggtcgtagtgtttgaagagcgtttaaacgctcttcgccccgcggaaatcgagcatcacggtttcgagacggccctga

SEQ ID NO: 159 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctggccatttatagtgttgaccgtttgnnngcaggtgcaatgcggcagacgaagaaaaggccacattgcacctgcnntatgcagcgctaaaaacggaccgttcgtttgaagagcgtttaaacgctcttcgccccaccgcgagctaatccgcgagtttcgagacggccctga

SEQ ID NO: 160 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctgaggcggtcaggatggctgcgtttgnnngcaggtgcaatgtaccttcccaccctactggaccattgcacctgcnntatgcacggcataactacgctcgctcgtttgaagagcgtttaaacgctcttcgcccgtcaacaccccgcgcgaatagtttcgagacggccctga

SEQ ID NO: 161 - DNA, 236 bp, artificial
  oligo
  misc_feature (93)..(95): n is a, c, g, or t
  misc_feature (141)..(142): n is a, c, g, or t
gaccgcgtctcacaccgcgtcaccaagccattgtaccgtttggagaccgtttaaacggtctctacctcctatcatacgtatcctggtgtttgnnngcaggtgcaatgacctacctctcaagtaccggccattgcacctgcnntatgcacgtaccgagaagtagggccggtttgaagagcgtttaaacgctcttcgcccgctacggtagacgtcgacaagtttcgagacggccctga

SEQ ID NO: 162 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctctgaaattagacatcaaagcgtttgnnngcaggtgcaatgccacactataaggcgtcacaccattgcacctgcnntatgcacgatacatgttcgtcggtaggtttgaagagcgtttaaacgctcttcgcccggtacgcgtaccatagtcgggtttcgagacggccctga

SEQ ID NO: 163 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctcaaagacctgcggcgagggcgtttgnnngcaggtgcaatgccccgacttaatgcaagagcgcattgcacctgcnntatgcatatcgactactacgaccgccgtttgaagagcgtttaaacgctcttcgcccctcgatcccgttcgacggccgtttcgagacggccctga

SEQ ID NO: 164 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctcatcggcctgacccccatgcgtttgnnngcaggtgcaatgagtatgccccagaccgatcaccattgcacctgcnntatgcatatcgcgaagtgccgtgagtgtttgaagagcgtttaaacgctcttcgccctcggtactttgaaaccgcgcgtttcgagacggccctga

SEQ ID NO: 165 - DNA, 236 bp, artificial
  oligo
  misc_feature (93)..(95): n is a, c, g, or t
  misc_feature (141)..(142): n is a, c, g, or t
gaccgcgtctcacaccggattccttccaataacacaagtttggagaccgtttaaacggtctctacctgttcgcgaagtgcccctagtgtttgnnngcaggtgcaatgagaacgggccgaagaatcgaacattgcacctgcnntatgcactcgcgcgcttagacaatttgtttgaagagcgtttaaacgctcttcgccccgcgacatgatggtacgcgtgtttcgagacggccctga

SEQ ID NO: 166 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctattgcgcgtttgaccgaatggtttgnnngcaggtgcaatgctctttcgcaggcccacaatacattgcacctgcnntatgcaccgaagtgtatcccgatccggtttgaagagcgtttaaacgctcttcgcccaatacattcgagcggcgtcggtttcgagacggccctga

SEQ ID NO: 167 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcgggtgcgtttactcgtctagtttgnnngcaggtgcaatgctaattcgtcgcaccatcgagcattgcacctgcnntatgcatcggtgcttcgattgtcggggtttgaagagcgtttaaacgctcttcgccccgcgcgcacataatgccttagtttcgagacggccctga

SEQ ID NO: 168 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctcgcattgaccgacccgtcgagtttgnnngcaggtgcaatgcctcacttacggaaaccccgacattgcacctgcnntatgcaatattcctcgctccgcgtaagtttgaagagcgtttaaacgctcttcgccctatcgcaatcgctccggaatgtttcgagacggccctga

SEQ ID NO: 169 - DNA, 236 bp, artificial
  oligo
  misc_feature (93)..(95): n is a, c, g, or t
  misc_feature (141)..(142): n is a, c, g, or t
gaccgcgtctcacaccggttacactctatctacacctgtttggagaccgtttaaacggtctctacctaatcttatcgattagcgggcgtttgnnngcaggtgcaatgcactcgctaaacgcagcactccattgcacctgcnntatgcatatggccgatgcaacgcaacgtttgaagagcgtttaaacgctcttcgcccggccccatatcggtacggcagtttcgagacggccctga

SEQ ID NO: 170 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctagtgaacggttgggtacgccgtttgnnngcaggtgcaatggcaacatgtcagtacccgttccattgcacctgcnntatgcagcgcgacgattatcacgttcgtttgaagagcgtttaaacgctcttcgccctccgcggaacatacctacgtgtttcgagacggccctga

SEQ ID NO: 171 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgagaagcactatgggggcatgtttgnnngcaggtgcaatgtgggtagagacttacaacgcccattgcacctgcnntatgcagtagaccaggcacccaacctgtttgaagagcgtttaaacgctcttcgcccaaccccaacatcagcaagcggtttcgagacggccctga

SEQ ID NO: 172 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctcaagggcgtgaacagcgacggtttgnnngcaggtgcaatgcctataccaacctcggcatcgcattgcacctgcnntatgcagtgcatacgtacttcgtccagtttgaagagcgtttaaacgctcttcgcccccacagtactcacttggtccgtttcgagacggccctga

SEQ ID NO: 173 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctatcttgcagatgctaatagcgtttgnnngcaggtgcaatgcaacctagacaagacctcccgcattgcacctgcnntatgcagcagtgaccgtcacgacctggtttgaagagcgtttaaacgctcttcgcccaacatgactccccggaggccgtttcgagacggccctga

SEQ ID NO: 174 - DNA, 236 bp, artificial
  oligo
  misc_feature (93)..(95): n is a, c, g, or t
  misc_feature (141)..(142): n is a, c, g, or t
gaccgcgtctcacaccggttgccaggtgagcccgctcgtttggagaccgtttaaacggtctctacctcaagggcgtgaacagcgacggtttgnnngcaggtgcaatgaccgtgacaaactgactagcccattgcacctgcnntatgcagtcacacctgagtaccactggtttgaagagcgtttaaacgctcttcgccctcccacagttgtaccttatcgtttcgagacggccctga

SEQ ID NO: 175 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgcagtgaccgtcacgacctggtttgnnngcaggtgcaatgtaacgacgaccaaggaccactcattgcacctgcnntatgcaccacagtactcacttggtccgtttgaagagcgtttaaacgctcttcgcccgcaaagccgatgtcagcgccgtttcgagacggccctga

SEQ ID NO: 176 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctgagaggtttccgaagactaagtttgnnngcaggtgcaatgtcggaaccacaacactgaccccattgcacctgcnntatgcataagctgctctctagacggagtttgaagagcgtttaaacgctcttcgcccatagcggggaacgcatcgtcgtttcgagacggccctga

SEQ ID NO: 177 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctgcaggtccttaccttgttccgtttgnnngcaggtgcaatgtgtgttaccacaatcgcgcgccattgcacctgcnntatgcaaagcactacccctgccaaccgtttgaagagcgtttaaacgctcttcgccccggccgttatcgagcaatatgtttcgagacggccctga

SEQ ID NO: 178 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgttcactacgagtgtattccgtttgnnngcaggtgcaatgacaccgacgacattaggaagccattgcacctgcnntatgcaaccaggctgacagttatcacgtttgaagagcgtttaaacgctcttcgcccatatggcggtcgaacaatgcgtttcgagacggccctga

SEQ ID NO: 179 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctaccttaccttgagactccaccctcgtttgnnngcaggtgcaatggcgcaggacatttacgcagctcattgcacctgcnntatgcaaaccccaacatcagcaagcggtttgaagagcgtttaaacgctcttcgcccccgtgacttacgctatacaggtttcgagacggccctga

SEQ ID NO: 180 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctaccaggcacccaacctcggcgtttgnnngcaggtgcaatgtctccatcgtgaaatcccgcacattgcacctgcnntatgcatggcattggggacctcgggtgtttgaagagcgtttaaacgctcttcgcccggtagcgcgttaaccttggtgtttcgagacggccctga

SEQ ID NO: 181 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctacctgtggtggttatggggcgtttgnnngcaggtgcaatgcctaaagtccgtcaaagaccccattgcacctgcnntatgcatcctcagctgccatatgtgcgtttgaagagcgtttaaacgctcttcgccctatgcggcgcggatcgtgacgtttcgagacggccctga

SEQ ID NO: 182 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctgcaaagccgatgtcagcgccgtttgnnngcaggtgcaatgttgacctcagcgatcaacgcccattgcacctgcnntatgcaaccaggcacccaacctcggcgtttgaagagcgtttaaacgctcttcgcccaaaccgcgacgcactcctaggtttcgagacggccctga

SEQ ID NO: 183 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctcagcttcaggaccccgatccgtttgnnngcaggtgcaatgcgagatgtgctaccaacctcgcattgcacctgcnntatgcagcttgtggtagatagcaacggtttgaagagcgtttaaacgctcttcgcccttgcccgcccgatactgcgagtttcgagacggccctga

SEQ ID NO: 184 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctccaagtaccttctccagcatgtttgnnngcaggtgcaatgatgcgccaccaaagcacgatacattgcacctgcnntatgcaaaagcagtcgcccctgcttggtttgaagagcgtttaaacgctcttcgccctagaccgtcatcggaatccggtttcgagacggccctga

SEQ ID NO: 185 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctgagaagcactatgggggcatgtttgnnngcaggtgcaatgtgttatccccgttcacaccgacattgcacctgcnntatgcatcacacttggagttacagcagtttgaagagcgtttaaacgctcttcgcccttaacacgtcccggcgccgtgtttcgagacggccctga

SEQ ID NO: 186 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctgctgacatcggctttgctgcgtttgnnngcaggtgcaatgttgcatgaccacactctgacgcattgcacctgcnntatgcacgatcgtgtggctcgtcgaggtttgaagagcgtttaaacgctcttcgccccgtagcggggcgaacttttagtttcgagacggccctga

SEQ ID NO: 187 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgttcactacgagtgtattccgtttgnnngcaggtgcaatgtccacgtactatctcgtcccccattgcacctgcnntatgcacgtcctacgacccgtggcgcgtttgaagagcgtttaaacgctcttcgcccaaccggcggtacgcgcgtttgtttcgagacggccctga

SEQ ID NO: 188 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctaccttaccttgagactccaccctcgtttgnnngcaggtgcaatggcctctaacactcactggaagcattgcacctgcnntatgcagtcccgcaccgacctttaatgtttgaagagcgtttaaacgctcttcgccccgctcgagaacacgtacatcgtttcgagacggccctga

SEQ ID NO: 189 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctgagaggtttccgaagactaagtttgnnngcaggtgcaatgagattcctctgccttatcccccattgcacctgcnntatgcatctcaacgtagcgcgacatcgtttgaagagcgtttaaacgctcttcgccctccactcgaacgagcggggtgtttcgagacggccctga

SEQ ID NO: 190 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctgtgcatacgtacttcgtccagtttgnnngcaggtgcaatgcttacacgtacagccactcgccattgcacctgcnntatgcagcgacgttagccctatcgcggtttgaagagcgtttaaacgctcttcgccctaccctcggcattcgaccgagtttcgagacggccctga

SEQ ID NO: 191 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctaccttgtgaaaaggactgcaggccgtttgnnngcaggtgcaatgcccatggatcattatccggcacattgcacctgcnntatgcacgccgaatattagcccgaacgtttgaagagcgtttaaacgctcttcgcccccacgccgaacgccgtcagtgtttcgagacggccctga

SEQ ID NO: 192 - DNA, 236 bp, artificial
  oligo
  misc_feature (93)..(95): n is a, c, g, or t
  misc_feature (141)..(142): n is a, c, g, or t
gaccgcgtctcacaccgtacgctgcaaagtctctggcgtttggagaccgtttaaacggtctctaccttaagctgctctctagacggagtttgnnngcaggtgcaatgcgcatcacatatctaggtccgcattgcacctgcnntatgcacggctatcggatagactagggtttgaagagcgtttaaacgctcttcgccccgacgacctacaatggcggtgtttcgagacggccctga

SEQ ID NO: 193 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaccaggcacccaacctcggcgtttgnnngcaggtgcaatgcctccttcaataaacgcgcaccattgcacctgcnntatgcacgcgataagatttcgagcacgtttgaagagcgtttaaacgctcttcgccccaatatcccggcgcgaagcggtttcgagacggccctga

SEQ ID NO: 194 - DNA, 236 bp, artificial
  oligo
  misc_feature (93)..(95): n is a, c, g, or t
  misc_feature (141)..(142): n is a, c, g, or t
gaccgcgtctcacaccgtcacacttggagttacagcagtttggagaccgtttaaacggtctctacctgcttgtggtagatagcaacggtttgnnngcaggtgcaatgcaaatccgaccaagcggtactcattgcacctgcnntatgcagacgccttaatcatcgagatgtttgaagagcgtttaaacgctcttcgcccgttccgtcgttcctcgcgtcgtttcgagacggccctga

SEQ ID NO: 195 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctctagcaagccataacaaaacgtttgnnngcaggtgcaatgatgcccgatccttaccacatccattgcacctgcnntatgcagctcgcgcattcttacgtcggtttgaagagcgtttaaacgctcttcgccccgatcgccccttttcggtaagtttcgagacggccctga

SEQ ID NO: 196 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgcgaggttcgccgtcgtgatgtttgnnngcaggtgcaatgcccattacccgcacttattgccattgcacctgcnntatgcacggatgttacctcggtagacgtttgaagagcgtttaaacgctcttcgccctcgtgtacactgatccggccgtttcgagacggccctga

SEQ ID NO: 197 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctaccttaccatccgacgcgctcgaagtttgnnngcaggtgcaatgtcatcgggaccacaacaaacgcattgcacctgcnntatgcaactcgttcgaccgcgtcgcggtttgaagagcgtttaaacgctcttcgcccgaccgcgtagcaattcgtatgtttcgagacggccctga

SEQ ID NO: 198 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctcgccaagtggattgccgtacgtttgnnngcaggtgcaatggaattcacagcccatccccttcattgcacctgcnntatgcatggaacggtcgacttcgggcgtttgaagagcgtttaaacgctcttcgcccctctcggcaattcgcgctcagtttcgagacggccctga

SEQ ID NO: 199 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctacctcggttcgaccgttacgttgtgtttgnnngcaggtgcaatgccgcatttcaaacgccttcaccattgcacctgcnntatgcatcgcgctccgtcgttttggtgtttgaagagcgtttaaacgctcttcgcccgtacgtttgtaactcggtaggtttcgagacggccctga

SEQ ID NO: 200 - DNA, 236 bp, artificial
  oligo
  misc_feature (93)..(95): n is a, c, g, or t
  misc_feature (141)..(142): n is a, c, g, or t
gaccgcgtctcacaccgatccccgtcgcagttgaactgtttggagaccgtttaaacggtctctacctatcagttcgagcgtgcgatggtttgnnngcaggtgcaatgactagcccctatacggaaccacattgcacctgcnntatgcaatattgccggcacgatagatgtttgaagagcgtttaaacgctcttcgccccccgaacggaataccccgtagtttcgagacggccctga

SEQ ID NO: 201 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctaagaggcttcttatgtcggcgtttgnnngcaggtgcaatgttcctcagagcaacgcctcttcattgcacctgcnntatgcatcgacaacccctacatcgtggtttgaagagcgtttaaacgctcttcgcccctcacggcagcgcacgcacagtttcgagacggccctga

SEQ ID NO: 202 - DNA, 236 bp, artificial
  oligo
  misc_feature (93)..(95): n is a, c, g, or t
  misc_feature (141)..(142): n is a, c, g, or t
gaccgcgtctcacaccggcatccacattcttacaggcgtttggagaccgtttaaacggtctctacctttctcggaagtcgaggacaggtttgnnngcaggtgcaatgaacaccgcagctataacagcccattgcacctgcnntatgcaaacggcacgtacgccatcgcgtttgaagagcgtttaaacgctcttcgccccgccggcgctctcaagtatcgtttcgagacggccctga

SEQ ID NO: 203 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctactcacggcagcgcacgcacgtttgnnngcaggtgcaatgtaatccaccagcggcacggaacattgcacctgcnntatgcacgcccagggagcgcaaacacgtttgaagagcgtttaaacgctcttcgcccgaactgtatacggctccgcggtttcgagacggccctga

SEQ ID NO: 204 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctaccttatccggttacctcatagtcgtttgnnngcaggtgcaatgttctagcccaaccctatctcccattgcacctgcnntatgcatcgggggacggttatttctggtttgaagagcgtttaaacgctcttcgcccatagccttgcgtcgagtatggtttcgagacggccctga

SEQ ID NO: 205 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctgtgacccgaggaaccggtgcgtttgnnngcaggtgcaatgcccccacatcattatagccctcattgcacctgcnntatgcacgcgcaccatagcatcacgcgtttgaagagcgtttaaacgctcttcgcccacgtgaaatcgatgccggatgtttcgagacggccctga

SEQ ID NO: 206 - DNA, 236 bp, artificial
  oligo
  misc_feature (93)..(95): n is a, c, g, or t
  misc_feature (141)..(142): n is a, c, g, or t
gaccgcgtctcacaccgctcacggcagcgcacgcacagtttggagaccgtttaaacggtctctacctcgcgcaccatagcatcacgcgtttgnnngcaggtgcaatgttatttgcccagacacctcggcattgcacctgcnntatgcatcgaacgcgaagggtcgcgtgtttgaagagcgtttaaacgctcttcgccccgacgccgacggccgatgcagtttcgagacggccctga

SEQ ID NO: 207 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctctcgtagcttgccacactgtgtttgnnngcaggtgcaatgaccctacctcaagaatgtcggcattgcacctgcnntatgcatcgtaatagatccgcgtctagtttgaagagcgtttaaacgctcttcgccctcgatagttgacccgttcaagtttcgagacggccctga

SEQ ID NO: 208 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctctcacggcagcgcacgcacagtttgnnngcaggtgcaatggcaccttccagaatgttcccacattgcacctgcnntatgcatatggcgcgctcgattcagggtttgaagagcgtttaaacgctcttcgcccctacgggtacatacgcgcgtgtttcgagacggccctga

SEQ ID NO: 209 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctgaagcgagagaatctcctcggtttgnnngcaggtgcaatgataaccatccacacacgggagcattgcacctgcnntatgcaccgcggcggataacgtgtttgtttgaagagcgtttaaacgctcttcgcccggccctattagcgtcgcgacgtttcgagacggccctga

SEQ ID NO: 210 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctcttctatggcagcatctcgcgtttgnnngcaggtgcaatgcacaagccaaaacatccgacccattgcacctgcnntatgcaggtcgtaattatgcccttcggtttgaagagcgtttaaacgctcttcgcccacgtcgacccttcgacgcttgtttcgagacggccctga

SEQ ID NO: 211 - DNA, 236 bp, artificial
  oligo
  misc_feature (93)..(95): n is a, c, g, or t
  misc_feature (141)..(142): n is a, c, g, or t
gaccgcgtctcacaccgggggcacgatggcctttaccgtttggagaccgtttaaacggtctctacctgtgacccgaggaaccggtgcgtttgnnngcaggtgcaatgcggctactcacaattcaggtgcattgcacctgcnntatgcaataagtccaatcgggcgccagtttgaagagcgtttaaacgctcttcgccctaaaacgccgcgtcggaatggtttcgagacggccctga

SEQ ID NO: 212 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctaaacgggacaacggccgcgtgtttgnnngcaggtgcaatggcacgtagacggacctatagccattgcacctgcnntatgcagcgagtcgaccgttggacgtgtttgaagagcgtttaaacgctcttcgcccggaacgcaatcgccggttgagtttcgagacggccctga

SEQ ID NO: 213 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctcgaacgatatccgaatgcacgtttgnnngcaggtgcaatgccgctgtttatatcaccgacgcattgcacctgcnntatgcaccgcccacgatattggcggagtttgaagagcgtttaaacgctcttcgccctcgcttcgttcgcacgtattgtttcgagacggccctga

SEQ ID NO: 214 - DNA, 236 bp, artificial
  oligo
  misc_feature (93)..(95): n is a, c, g, or t
  misc_feature (141)..(142): n is a, c, g, or t
gaccgcgtctcacaccgttctcggaagtcgaggacaggtttggagaccgtttaaacggtctctacctgagcgcgtcggtatgcgtcggtttgnnngcaggtgcaatgcgcactcacctcacgtttaaccattgcacctgcnntatgcatgacgttcaccgaatacgacgtttgaagagcgtttaaacgctcttcgcccctttccttcgcgcgtaatcggtttcgagacggccctga

SEQ ID NO: 215 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctaccttagtggtcgcctgcgcgaatgtttgnnngcaggtgcaatggcctcttgaccacgaactatgcattgcacctgcnntatgcacgcgaggcgtttagcctagtgtttgaagagcgtttaaacgctcttcgccctacgcacgaggatcgcctatgtttcgagacggccctga

SEQ ID NO: 216 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctcaatacagggatacccactagtttgnnngcaggtgcaatggcagaaatcatatcggccacccattgcacctgcnntatgcaacctatcatacgtatcctgggtttgaagagcgtttaaacgctcttcgcccacacaacactgatgaggtccgtttcgagacggccctga

SEQ ID NO: 217 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctttgttacagccttattacacgtttgnnngcaggtgcaatgccggacacagaaaactccgtacattgcacctgcnntatgcaggccatttatagtgttgaccgtttgaagagcgtttaaacgctcttcgccctcatcctgcatgctagaccagtttcgagacggccctga

SEQ ID NO: 218 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctcaacttcttcgtggtatctcgtttgnnngcaggtgcaatggagccactaggaaatcttccccattgcacctgcnntatgcatcatcctgcatgctagaccagtttgaagagcgtttaaacgctcttcgccccagcttgtccaactggtcgggtttcgagacggccctga

SEQ ID NO: 219 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctaccttgttgggcagctgtatgatcgtttgnnngcaggtgcaatgcaccgtcagcactttaatcgccattgcacctgcnntatgcatctttgaaccattgtgttatgtttgaagagcgtttaaacgctcttcgcccattcttctctgtgaatgtcggtttcgagacggccctga

SEQ ID NO: 220 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctagatcatacagctgcccaacgtttgnnngcaggtgcaatgaccacgccgagcatttatcctcattgcacctgcnntatgcaggaagggacactaacctggtgtttgaagagcgtttaaacgctcttcgcccactttccatgtcagccatacgtttcgagacggccctga

SEQ ID NO: 221 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcagcttgtccaactggtcgggtttgnnngcaggtgcaatgtccatgaacagtcccatgccacattgcacctgcnntatgcaggagagccaacccctaccaggtttgaagagcgtttaaacgctcttcgcccttgggactcctcggttatttgtttcgagacggccctga

SEQ ID NO: 222 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctacctggactgagagctgttgacacgtttgnnngcaggtgcaatgcgacttgtacaacgatcccgacattgcacctgcnntatgcactggaaggtgaggcctaggcgtttgaagagcgtttaaacgctcttcgcccgaggcggtcaggatggctgcgtttcgagacggccctga

SEQ ID NO: 223 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctgattccttccaataacacaagtttgnnngcaggtgcaatgacctcagcacagacagtgacccattgcacctgcnntatgcacctatcatacgtatcctggtgtttgaagagcgtttaaacgctcttcgccctgcccacaaagtatggcggtgtttcgagacggccctga

SEQ ID NO: 224 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctggagagccaacccctaccaggtttgnnngcaggtgcaatggacctcacggcatcacaacagcattgcacctgcnntatgcattgttacagccttattacacgtttgaagagcgtttaaacgctcttcgcccgattccttccaataacacaagtttcgagacggccctga

SEQ ID NO: 225 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctacaggtgacccaaccttcaggtttgnnngcaggtgcaatgcctttcacattccggccaactcattgcacctgcnntatgcagccctttagctggtatctgcgtttgaagagcgtttaaacgctcttcgcccggagagccaacccctaccaggtttcgagacggccctga

SEQ ID NO: 226 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctgtatcctggtgggatttacagtttgnnngcaggtgcaatgtgtgatggggtggacctgttgcattgcacctgcnntatgcagctcaaaccattacagaagggtttgaagagcgtttaaacgctcttcgcccaatcccttgccccagcacatgtttcgagacggccctga

SEQ ID NO: 227 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctctccacaaagacctgcggcggtttgnnngcaggtgcaatgccagcaggccgtatgtatagtcattgcacctgcnntatgcaggccatttatagtgttgaccgtttgaagagcgtttaaacgctcttcgcccacaggtgacccaaccttcaggtttcgagacggccctga

SEQ ID NO: 228 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctctaaagggcgatctcaacaagtttgnnngcaggtgcaatggccaatcgtccgactaaacgccattgcacctgcnntatgcacatcggcctgacccccatgcgtttgaagagcgtttaaacgctcttcgccctcacccataggagccacagcgtttcgagacggccctga

SEQ ID NO: 229 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctctgctagccaacaccactgagtttgnnngcaggtgcaatgtcacccggagttcactacccacattgcacctgcnntatgcagttacactctatctacacctgtttgaagagcgtttaaacgctcttcgcccgctcaaaccattacagaagggtttcgagacggccctga

SEQ ID NO: 230 - DNA, 236 bp, artificial
  oligo
  misc_feature (93)..(95): n is a, c, g, or t
  misc_feature (141)..(142): n is a, c, g, or t
gaccgcgtctcacaccgcgtcaccaagccattgtaccgtttggagaccgtttaaacggtctctacctctggaaggtgaggcctaggcgtttgnnngcaggtgcaatgatgcatacaccgtcgctaaggcattgcacctgcnntatgcaattcttctctgtgaatgtcggtttgaagagcgtttaaacgctcttcgcccagctgtagtagagtccccatgtttcgagacggccctga

SEQ ID NO: 231 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctccctcaccttctacccagccgtttgnnngcaggtgcaatgtccctcgaaaatccccacaagcattgcacctgcnntatgcacagtgcaacttactattacggtttgaagagcgtttaaacgctcttcgcccgtgcgatagaccgtcccgatgtttcgagacggccctga

SEQ ID NO: 232 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctcaatacagggatacccactagtttgnnngcaggtgcaatgctaaaccgatagacgtacccccattgcacctgcnntatgcaacatagacccctgttgtaaggtttgaagagcgtttaaacgctcttcgccctcgatagttgcgtcgcatacgtttcgagacggccctga

SEQ ID NO: 233 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctaccttctttgaaccattgtgttatgtttgnnngcaggtgcaatggaaatatcagcgtccgctcaccattgcacctgcnntatgcatgttgagatcgccctttagcgtttgaagagcgtttaaacgctcttcgccccgcgaaaacggctgtgcgtagtttcgagacggccctga

SEQ ID NO: 234 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttctttgaaccattgtgttatgtttgnnngcaggtgcaatgccccattgtaatgaccatccccattgcacctgcnntatgcacgtcaccaagccattgtaccgtttgaagagcgtttaaacgctcttcgcccggcgctcgataatactgtttgtttcgagacggccctga

SEQ ID NO: 235 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctaccttcacccataggagccacagcgtttgnnngcaggtgcaatggagaaacgttctgtccccaagcattgcacctgcnntatgcatcatcctgcatgctagaccagtttgaagagcgtttaaacgctcttcgccccgcgaaaagcgtccgatgatgtttcgagacggccctga

SEQ ID NO: 236 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctcagtgcaacttactattacggtttgnnngcaggtgcaatgcagacgaaagacccactgaagcattgcacctgcnntatgcatgcaagccttacctgtagcggtttgaagagcgtttaaacgctcttcgcccgcggtcgatgcgcaaacgacgtttcgagacggccctga

SEQ ID NO: 237 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttgggactcctcggttatttagtttgnnngcaggtgcaatgctattcccctcgagtttccaccattgcacctgcnntatgcacaacttcttcgtggtatctcgtttgaagagcgtttaaacgctcttcgcccgtcctaagccggtattcgatgtttcgagacggccctga

SEQ ID NO: 238 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctatcagttctgagcaactcgtgtttgnnngcaggtgcaatggcctaccggagatataagacgcattgcacctgcnntatgcatcatcctgcatgctagaccagtttgaagagcgtttaaacgctcttcgccctcgagacaaattcgtcgcgtgtttcgagacggccctga

SEQ ID NO: 239 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctcgtcaccaagccattgtaccgtttgnnngcaggtgcaatgcccgacttcactagttagacgcattgcacctgcnntatgcacagtgcaacttactattacggtttgaagagcgtttaaacgctcttcgcccagcttcgttacggttaaacggtttcgagacggccctga

SEQ ID NO: 240 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcaaagacctgcggcgagggcgtttgnnngcaggtgcaatggtagagaagcaacggcaaggacattgcacctgcnntatgcactaaagggcgatctcaacaagtttgaagagcgtttaaacgctcttcgccctgtcgcggcgagtatcaagagtttcgagacggccctga

SEQ ID NO: 241 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctctggaaggtgaggcctaggcgtttgnnngcaggtgcaatggactcaggacaactattccggcattgcacctgcnntatgcatgtgatggtgaatattcacagtttgaagagcgtttaaacgctcttcgccctttcgtcggcaatcgtgcgcgtttcgagacggccctga

SEQ ID NO: 242 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctgtatcctggtgggatttacagtttgnnngcaggtgcaatggcgattcaacgatcagatgcccattgcacctgcnntatgcagttacactctatctacacctgtttgaagagcgtttaaacgctcttcgcccaacacacgtagcgttacgcggtttcgagacggccctga

SEQ ID NO: 243 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctgctcaaaccattacagaagggtttgnnngcaggtgcaatgctgaaatctgcgaccaccgtacattgcacctgcnntatgcaggagagccaacccctaccaggtttgaagagcgtttaaacgctcttcgcccgcgccgaaaaacgtcggttggtttcgagacggccctga

SEQ ID NO: 244 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctgctcaaaccattacagaagggtttgnnngcaggtgcaatgccccgacatataatcttcggccattgcacctgcnntatgcatgtgatggtgaatattcacagtttgaagagcgtttaaacgctcttcgccctccgcaacgcttaacgtgtagtttcgagacggccctga

SEQ ID NO: 245 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctggactgagagctgttgacacgtttgnnngcaggtgcaatgtccgtttccacgcctaaacaccattgcacctgcnntatgcatctgggcatgtgggtccggcgtttgaagagcgtttaaacgctcttcgcccgcgcgcaataacctacccgggtttcgagacggccctga

SEQ ID NO: 246 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctctaaagggcgatctcaacaagtttgnnngcaggtgcaatgcgcaaatgcacgcttcgagatcattgcacctgcnntatgcacaatacagggatacccactagtttgaagagcgtttaaacgctcttcgccccgtatcgtatctgacgtgtggtttcgagacggccctga

SEQ ID NO: 247 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctaatcccttgccccagcacatgtttgnnngcaggtgcaatggcatcaatgcatagcacggcacattgcacctgcnntatgcactggaaggtgaggcctaggcgtttgaagagcgtttaaacgctcttcgcccaaacggtcggcccggcagtcgtttcgagacggccctga

SEQ ID NO: 248 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctaccttgtgatggtgaatattcacagtttgnnngcaggtgcaatgcggcagctagcataacaaagccattgcacctgcnntatgcactccacaaagacctgcggcggtttgaagagcgtttaaacgctcttcgccctgtcgtatgccgcttaacgcgtttcgagacggccctga

SEQ ID NO: 249 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctcgtcaccaagccattgtaccgtttgnnngcaggtgcaatgaacacacctttaacgtgcccccattgcacctgcnntatgcatgcaagccttacctgtagcggtttgaagagcgtttaaacgctcttcgcccggaacgtgtcgcgcattctagtttcgagacggccctga

SEQ ID NO: 250 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctagaagttggtgacgttctgcgtttgnnngcaggtgcaatgaccgtttaacagcccgccatccattgcacctgcnntatgcactgctagccaacaccactgagtttgaagagcgtttaaacgctcttcgccccgcacatccggcgtaggtatgtttcgagacggccctga

SEQ ID NO: 251 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgggactcctcggttatttagtttgnnngcaggtgcaatgaccacgacggaatctctcagacattgcacctgcnntatgcaacgtagatgccgttcggatagtttgaagagcgtttaaacgctcttcgccccgtcgtgctcgcacctgtaagtttcgagacggccctga

SEQ ID NO: 252 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctctggaaggtgaggcctaggcgtttgnnngcaggtgcaatgccgtagaaacatatcgcgcgtcattgcacctgcnntatgcactataccgatacggagtgacgtttgaagagcgtttaaacgctcttcgccccgatttgatattaccgcgatgtttcgagacggccctga

SEQ ID NO: 253 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctggaagggacactaacctggtgtttgnnngcaggtgcaatgtgatgtagcattagacgcccccattgcacctgcnntatgcagtaactcgtatacgcgcaaggtttgaagagcgtttaaacgctcttcgcccttaacatcgcacgtcgacgggtttcgagacggccctga

SEQ ID NO: 254 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctagctgtagtagagtccccatgtttgnnngcaggtgcaatgtccattaggcaaaacgaggcccattgcacctgcnntatgcagttgtgtcgcatcgacgagtgtttgaagagcgtttaaacgctcttcgcccacgccgagagttgttatgcggtttcgagacggccctga

SEQ ID NO: 255 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctgattccttccaataacacaagtttgnnngcaggtgcaatgccgacacccgtacattcacttcattgcacctgcnntatgcacggaacagacacgtccgaacgtttgaagagcgtttaaacgctcttcgcccataatgggcggtcgtaccgtgtttcgagacggccctga

SEQ ID NO: 256 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctcccaccaggatacgtatgatgtttgnnngcaggtgcaatgccctttcaatagcgtacccctcattgcacctgcnntatgcacgccgcacggaattcgcaaggtttgaagagcgtttaaacgctcttcgccccctcatgaccgctcgccgatgtttcgagacggccctga

SEQ ID NO: 257 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctaccttgttgggcagctgtatgatcgtttgnnngcaggtgcaatggatatttgacccatcggcacccattgcacctgcnntatgcagcaatctacgacatcccggcgtttgaagagcgtttaaacgctcttcgccccttacgcacggatacggcgagtttcgagacggccctga

SEQ ID NO: 258 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttgttgagatcgccctttagcgtttgnnngcaggtgcaatgcccagacaaagcctttggacccattgcacctgcnntatgcaagtcctacgacgcgccgcaagtttgaagagcgtttaaacgctcttcgccctagtggatccgtacatacgcgtttcgagacggccctga

SEQ ID NO: 259 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctcagcttgtccaactggtcgggtttgnnngcaggtgcaatgtactcgactaactcacggcgccattgcacctgcnntatgcagtgctataactctcgcgcgggtttgaagagcgtttaaacgctcttcgcccccggcgcttctagaccgaatgtttcgagacggccctga

SEQ ID NO: 260 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctacaggtgacccaaccttcaggtttgnnngcaggtgcaatgtgcagcatgccacatagtagccattgcacctgcnntatgcatatccggcccggaaggtcgtgtttgaagagcgtttaaacgctcttcgcccctcgtcgacttacacgctaggtttcgagacggccctga

SEQ ID NO: 261 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctggccatttatagtgttgaccgtttgnnngcaggtgcaatgcaccctcggatacatatcgcgcattgcacctgcnntatgcacgctcgtggctacgacgattgtttgaagagcgtttaaacgctcttcgcccgatctaacgcaacctacgttgtttcgagacggccctga

SEQ ID NO: 262 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctcatcggcctgacccccatgcgtttgnnngcaggtgcaatgtatccaggaatcaagccagcgcattgcacctgcnntatgcatcgtccgactcaacacgatggtttgaagagcgtttaaacgctcttcgccctacagtcgccggtcgtattagtttcgagacggccctga

SEQ ID NO: 263 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctacatagacccctgttgtaaggtttgnnngcaggtgcaatgtcgaaccctgcccaccaattacattgcacctgcnntatgcaacatcttaggttatgcggcggtttgaagagcgtttaaacgctcttcgcccaaaccgatcgcgagtgcgcggtttcgagacggccctga

SEQ ID NO: 264 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctcatcggcctgacccccatgcgtttgnnngcaggtgcaatgcgttccgtacacaatccactgcattgcacctgcnntatgcactttaccgcgcgacggacaagtttgaagagcgtttaaacgctcttcgcccgcgtcgttttgcgcgattaggtttcgagacggccctga

SEQ ID NO: 265 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctctgaaattagacatcaaagcgtttgnnngcaggtgcaatgaaaatcgtcgcacgcccttctcattgcacctgcnntatgcacggcatcacaacttcgtgcggtttgaagagcgtttaaacgctcttcgccccgcatgggctcggttgcgacgtttcgagacggccctga

SEQ ID NO: 266 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctacggaccttccgaacccgacgtttgnnngcaggtgcaatgcggacaacaccttgccttcttcattgcacctgcnntatgcattacgcgtacctacgcctatgtttgaagagcgtttaaacgctcttcgcccccgacgatctaaaccgaccggtttcgagacggccctga

SEQ ID NO: 267 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctaccttaagcgacgccgatcataccgtttgnnngcaggtgcaatgccacacgagaaccacaatagccattgcacctgcnntatgcaacgagtcgtcatcgtgccgtgtttgaagagcgtttaaacgctcttcgcccgcccatagcgacggtgatcggtttcgagacggccctga

SEQ ID NO: 268 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctatcgaccggttgatcctcgagtttgnnngcaggtgcaatgaatctagcacgtgcccaacgtcattgcacctgcnntatgcatagtcgaccgcggacccgttgtttgaagagcgtttaaacgctcttcgccccagctcggtacaatcgcggcgtttcgagacggccctga

SEQ ID NO: 269 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctgttacgaccttttacccgcggtttgnnngcaggtgcaatgggaccacacaagtttaagcggcattgcacctgcnntatgcatgagtcgcacttgcggcgacgtttgaagagcgtttaaacgctcttcgccccggtaggatgtttaaccgtggtttcgagacggccctga

SEQ ID NO: 270 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctatcacggcgtaccttcatacgtttgnnngcaggtgcaatggcgaccgaatcagattgccagcattgcacctgcnntatgcacgcatttgtcgacggatgatgtttgaagagcgtttaaacgctcttcgccctttggccgcgcgctcgatacgtttcgagacggccctga

SEQ ID NO: 271 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctcgttagtgtgtcgcgcataagtttgnnngcaggtgcaatggcaacgtttgacaacgacacgcattgcacctgcnntatgcaggtcgctcgatgcacgacatgtttgaagagcgtttaaacgctcttcgcccacgaatatcacgacgtaatagtttcgagacggccctga

SEQ ID NO: 272 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgtcacacctgagtaccactggtttgnnngcaggtgcaatgcagtagccccaattcatcgtgcattgcacctgcnntatgcagtgcatacgtacttcgtccagtttgaagagcgtttaaacgctcttcgccctcacacttggagttacagcagtttcgagacggccctga

SEQ ID NO: 273 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctcagcttcaggaccccgatccgtttgnnngcaggtgcaatggcaaacctagactgaatcgcccattgcacctgcnntatgcagcaggtccttaccttgttccgtttgaagagcgtttaaacgctcttcgcccaaccccaacatcagcaagcggtttcgagacggccctga

SEQ ID NO: 274 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctgagaagcactatgggggcatgtttgnnngcaggtgcaatggcgaccatcaagatggctgaacattgcacctgcnntatgcatacgctgcaaagtctctggcgtttgaagagcgtttaaacgctcttcgccctaaccatagtacccagttgtgtttcgagacggccctga

SEQ ID NO: 275 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctgtgtctgtatgacccacccggtttgnnngcaggtgcaatggcaacatcaccacaagttccgcattgcacctgcnntatgcatcctcagctgccatatgtgcgtttgaagagcgtttaaacgctcttcgcccatccccgtcgcagttgaactgtttcgagacggccctga

SEQ ID NO: 276 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctaccttacgctgcaaagtctctggcgtttgnnngcaggtgcaatgacgagaacgtacaccaccaagcattgcacctgcnntatgcatgctgtcgtgagtgccagccgtttgaagagcgtttaaacgctcttcgcccgcaggtccttaccttgttccgtttcgagacggccctga

SEQ ID NO: 277 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttaagctgctctctagacggagtttgnnngcaggtgcaatgcggaccaacgttacccctaatcattgcacctgcnntatgcatcctgccgaggttgggtgccgtttgaagagcgtttaaacgctcttcgcccgaatctcgcgtcacgtaatcgtttcgagacggccctga

SEQ ID NO: 278 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgcaggtccttaccttgttccgtttgnnngcaggtgcaatggcttcatttccacagtagccccattgcacctgcnntatgcagcagtgaccgtcacgacctggtttgaagagcgtttaaacgctcttcgcccttgctagttacccgtcgcgagtttcgagacggccctga

SEQ ID NO: 279 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctaaagcagtcgcccctgcttggtttgnnngcaggtgcaatgcccttactagcaacgtatccccattgcacctgcnntatgcatgtgcttaagaccgggaccagtttgaagagcgtttaaacgctcttcgccctcgtctgaagcgcgctgtacgtttcgagacggccctga

SEQ ID NO: 280 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctagccacgcttgctgatgttggtttgnnngcaggtgcaatgtgtccttaacccacctgactccattgcacctgcnntatgcattattcttgtccaggcaagggtttgaagagcgtttaaacgctcttcgcccgttagcgtccgaaaacccgggtttcgagacggccctga

SEQ ID NO: 281 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctagccacgcttgctgatgttggtttgnnngcaggtgcaatgtgcgataactcataccagccgcattgcacctgcnntatgcaaacatgactccccggaggccgtttgaagagcgtttaaacgctcttcgcccccgcaacgttcacgatccacgtttcgagacggccctga

SEQ ID NO: 282 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctatcttgcagatgctaatagcgtttgnnngcaggtgcaatgtctgtacagcgctcaacacgccattgcacctgcnntatgcatacgctgcaaagtctctggcgtttgaagagcgtttaaacgctcttcgcccaacaagcggctatgtcggacgtttcgagacggccctga

SEQ ID NO: 283 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctcagcttcaggaccccgatccgtttgnnngcaggtgcaatggagagcctaacaccacaaagccattgcacctgcnntatgcagcagtgaccgtcacgacctggtttgaagagcgtttaaacgctcttcgcccaacttgcgtgcgacgaacacgtttcgagacggccctga

SEQ ID NO: 284 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctttcctacaaccttctcgcaagtttgnnngcaggtgcaatgcgaccttcgaccgcaacatttcattgcacctgcnntatgcacagcttcaggaccccgatccgtttgaagagcgtttaaacgctcttcgcccagactactagccccgtccgcgtttcgagacggccctga

SEQ ID NO: 285 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctcaagggcgtgaacagcgacggtttgnnngcaggtgcaatgtgagcaataaagcgccccgaacattgcacctgcnntatgcattattcttgtccaggcaagggtttgaagagcgtttaaacgctcttcgccccgttcgagccgcgtatccgtgtttcgagacggccctga

SEQ ID NO: 286 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgagaagcactatgggggcatgtttgnnngcaggtgcaatggtatccggacatcctagcgatcattgcacctgcnntatgcagagccgctgtgatcataccagtttgaagagcgtttaaacgctcttcgcccgtactggtgcgtccgtatctgtttcgagacggccctga

SEQ ID NO: 287 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctccaagtaccttctccagcatgtttgnnngcaggtgcaatgtacacctggaatcaagacccccattgcacctgcnntatgcacgacgttcgacaatgggatcgtttgaagagcgtttaaacgctcttcgccccgctcgatcagactaacgtagtttcgagacggccctga

SEQ ID NO: 288 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctagccacgcttgctgatgttggtttgnnngcaggtgcaatgcgcagaccacggtaatctagacattgcacctgcnntatgcagatccactcgcggaacttacgtttgaagagcgtttaaacgctcttcgccctcgacagactatctgccgtagtttcgagacggccctga

SEQ ID NO: 289 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgccatgggcaccttctccgcgtttgnnngcaggtgcaatggccactccgatattgtccctccattgcacctgcnntatgcatgtgtcgcgagggcgattaagtttgaagagcgtttaaacgctcttcgccccttctaacgtcgtgacgagagtttcgagacggccctga

SEQ ID NO: 290 - DNA, 236 bp, artificial
  oligo
  misc_feature (93)..(95): n is a, c, g, or t
  misc_feature (141)..(142): n is a, c, g, or t
gaccgcgtctcacaccgcaagggcgtgaacagcgacggtttggagaccgtttaaacggtctctacctacctgtggtggttatggggcgtttgnnngcaggtgcaatgcccatgctagatacggacgaacattgcacctgcnntatgcacgaaattcccgacgctcccggtttgaagagcgtttaaacgctcttcgcccggtcggacctctcggcggatgtttcgagacggccctga

SEQ ID NO: 291 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgctgacatcggctttgctgcgtttgnnngcaggtgcaatgcacggaacgattcgccctgaacattgcacctgcnntatgcacgtcacggcgagcgaaccccgtttgaagagcgtttaaacgctcttcgcccccgtggatcgtttccgcactgtttcgagacggccctga

SEQ ID NO: 292 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgtgcatacgtacttcgtccagtttgnnngcaggtgcaatgtatgcccattcacaggtccctcattgcacctgcnntatgcattaacgcgtggcatcgagatgtttgaagagcgtttaaacgctcttcgcccacgagccttcgcgccgtttcgtttcgagacggccctga

SEQ ID NO: 293 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctctagcaagccataacaaaacgtttgnnngcaggtgcaatgaagaacaacatcaggcgaccgcattgcacctgcnntatgcagtcgcgattcgacgtgataggtttgaagagcgtttaaacgctcttcgcccattcaacgacgaccgtgcgagtttcgagacggccctga

SEQ ID NO: 294 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctttattcttgtccaggcaagggtttgnnngcaggtgcaatgcctcaatcctagtcccacggacattgcacctgcnntatgcacgaattatccgagccgcaaggtttgaagagcgtttaaacgctcttcgcccacggcgaacggctagtcgccgtttcgagacggccctga

SEQ ID NO: 295 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctacctaccaggcacccaacctcggcgtttgnnngcaggtgcaatgcgatcagcatttatccggcaccattgcacctgcnntatgcacggcacgaatttacggcgcggtttgaagagcgtttaaacgctcttcgcccggcgtatccccgtacgctacgtttcgagacggccctga

SEQ ID NO: 296 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctgagaagcactatgggggcatgtttgnnngcaggtgcaatggcttaatcccaaggcatgtcccattgcacctgcnntatgcaccccgtcgcggtatctcgcagtttgaagagcgtttaaacgctcttcgccctcgctcgtcacgggatagtcgtttcgagacggccctga

SEQ ID NO: 297 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttactcgttgcggcgataccggtttgnnngcaggtgcaatggacacacgcctataacacgagcattgcacctgcnntatgcatcacgagctatacaacgcgtgtttgaagagcgtttaaacgctcttcgcccccgcctacgccatgccgattgtttcgagacggccctga

SEQ ID NO: 298 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctatcgcggggtcgaaccacgagtttgnnngcaggtgcaatgtgccaacggacgaatccactccattgcacctgcnntatgcaccgacgtgtcggtacgcgccgtttgaagagcgtttaaacgctcttcgcccatcgcgtcatcgcgaaacgtgtttcgagacggccctga

SEQ ID NO: 299 - DNA, 236 bp, artificial
  oligo
  misc_feature (93)..(95): n is a, c, g, or t
  misc_feature (141)..(142): n is a, c, g, or t
gaccgcgtctcacaccgcagcttcaggaccccgatccgtttggagaccgtttaaacggtctctacctgttcgaacgcagaatgtacggtttgnnngcaggtgcaatgatctcagcatcagcacgcagtcattgcacctgcnntatgcaaccgttcgacgagtttagtcgtttgaagagcgtttaaacgctcttcgcccgtagccggcccgacgcaaacgtttcgagacggccctga

SEQ ID NO: 300 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctatcgcgcgaacttctgagtagtttgnnngcaggtgcaatgatacttggttcgacccaacggcattgcacctgcnntatgcagatacggcggtaacgtatcggtttgaagagcgtttaaacgctcttcgcccctcaatgaacgacctaccgggtttcgagacggccctga

SEQ ID NO: 301 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctacctccgcggacttaagcgcgcaagtttgnnngcaggtgcaatgctcggataagtaaaccgcacccattgcacctgcnntatgcaacgagcgatagcctaggacggtttgaagagcgtttaaacgctcttcgcccccgtcatcgatcggtagctagtttcgagacggccctga

SEQ ID NO: 302 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctcattgtatacgccttcctgggtttgnnngcaggtgcaatggtacaggagaaaccagcaccacattgcacctgcnntatgcatcgggggacggttatttctggtttgaagagcgtttaaacgctcttcgcccagggcacgtcaactctacacgtttcgagacggccctga

SEQ ID NO: 303 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctgcgccagtgtttgcgctcccgtttgnnngcaggtgcaatgtcttcctcactgtcctagtcgcattgcacctgcnntatgcatgataagcacctggtgctccgtttgaagagcgtttaaacgctcttcgcccgtaaagtcggtccgaatcgcgtttcgagacggccctga

SEQ ID NO: 304 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctcaacggcacgtacgccatcggtttgnnngcaggtgcaatgcgatcacgacgtacagttcagcattgcacctgcnntatgcagccttgagcccggtggggctgtttgaagagcgtttaaacgctcttcgcccgtcctaccacggatatcggagtttcgagacggccctga

SEQ ID NO: 305 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctactgtcaggcagcaccactcgtttgnnngcaggtgcaatgcagcgttcaacctccttcctacattgcacctgcnntatgcaagggcacgtcaactctacacgtttgaagagcgtttaaacgctcttcgcccggttatccgtaagactccgggtttcgagacggccctga

SEQ ID NO: 306 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgggccagggtctgcatatgcgtttgnnngcaggtgcaatgccacatatgcggttacacagccattgcacctgcnntatgcagtgacccgaggaaccggtgcgtttgaagagcgtttaaacgctcttcgccccagcaatacgatccgcgcgtgtttcgagacggccctga

SEQ ID NO: 307 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctgcgccagtgtttgcgctcccgtttgnnngcaggtgcaatgccctcagtacaagacgaactccattgcacctgcnntatgcaccccgttaccggatcgtcgtgtttgaagagcgtttaaacgctcttcgcccccgcgcgtaaggtttcgtgcgtttcgagacggccctga

SEQ ID NO: 308 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctactcacggcagcgcacgcacgtttgnnngcaggtgcaatgtccatgcacccaaccttcctccattgcacctgcnntatgcaaaaggtccgagtatcgcgccgtttgaagagcgtttaaacgctcttcgccctgcggtcgcgggttctaatagtttcgagacggccctga

SEQ ID NO: 309 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctacctatcctccacgtgcggttcccgtttgnnngcaggtgcaatgccccaaaccattagtgttccgcattgcacctgcnntatgcaacgtccgtcggtatggacaagtttgaagagcgtttaaacgctcttcgcccggcgttagttggttacgcgcgtttcgagacggccctga

SEQ ID NO: 310 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctggccagaatacaggtcccgcgtttgnnngcaggtgcaatgttcagtccgacaccgaaagaccattgcacctgcnntatgcacgaagttgccgaatgcgaatgtttgaagagcgtttaaacgctcttcgccccgatagcgagttcgtgcgacgtttcgagacggccctga

SEQ ID NO: 311 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctacctgcgccagtgtttgcgctcccgtttgnnngcaggtgcaatgtcttcacagaacctcacggcacattgcacctgcnntatgcaatcgcgagcggtctacgtccgtttgaagagcgtttaaacgctcttcgcccccggtcgtcgcgttatcgtcgtttcgagacggccctga

SEQ ID NO: 312 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctgctcgggatccagagatggggtttgnnngcaggtgcaatgcaaacatgccccgtaccctaacattgcacctgcnntatgcacccgcgacaaatctgccgtagtttgaagagcgtttaaacgctcttcgccccgatacggtgcaccgattcagtttcgagacggccctga

SEQ ID NO: 313 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctccattaattggcgatgcgcggtttgnnngcaggtgcaatgcacacgatcatcaaccagccccattgcacctgcnntatgcactcgttacgccgctactaaggtttgaagagcgtttaaacgctcttcgccccggtacacgacttcgcccgcgtttcgagacggccctga

SEQ ID NO: 314 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctccggatcgtgatcctcggttgtttgnnngcaggtgcaatgctcgcctaacattgacctcggcattgcacctgcnntatgcaaccggttacgcgacttctatgtttgaagagcgtttaaacgctcttcgcccttcgtacggttggaccggttgtttcgagacggccctga

SEQ ID NO: 315 - DNA, 236 bp, artificial
  oligo
  misc_feature (93)..(95): n is a, c, g, or t
  misc_feature (141)..(142): n is a, c, g, or t
gaccgcgtctcacaccgagggcacgtcaactctacacgtttggagaccgtttaaacggtctctaccttaggtgtcggtccggattaggtttgnnngcaggtgcaatggcctctatcaaaccaacgccccattgcacctgcnntatgcatatcgattcgccccgcctgtgtttgaagagcgtttaaacgctcttcgcccgcgcacgctaccacttaggtgtttcgagacggccctga

SEQ ID NO: 316 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctgttcattgggtcggcgtcgtgtttgnnngcaggtgcaatgctccttaccagcctcatttgccattgcacctgcnntatgcacgcatacgacggaccgccgagtttgaagagcgtttaaacgctcttcgccctagccgtcgacggctattgggtttcgagacggccctga

SEQ ID NO: 317 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctactttccatgtcagccatacgtttgnnngcaggtgcaatgctcccaaaacgcgctgaaatccattgcacctgcnntatgcatgggactcctcggttatttagtttgaagagcgtttaaacgctcttcgccctcacccataggagccacagcgtttcgagacggccctga

SEQ ID NO: 318 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctttcatagacctaatgtgactgtttgnnngcaggtgcaatgcccatttcaaagcatgcgcgacattgcacctgcnntatgcatgaatgaccagggtacctgcgtttgaagagcgtttaaacgctcttcgcccgtatcctggtgggatttacagtttcgagacggccctga

SEQ ID NO: 319 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctaccttgttgggcagctgtatgatcgtttgnnngcaggtgcaatgcttgtccccaatcactggcgacattgcacctgcnntatgcacaggcacgatagatacaaaggtttgaagagcgtttaaacgctcttcgcccgattccttccaataacacaagtttcgagacggccctga

SEQ ID NO: 320 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctctgcagttgagctggcaatcgtttgnnngcaggtgcaatgcccctcgactctatctaagtgcattgcacctgcnntatgcatgggactcctcggttatttagtttgaagagcgtttaaacgctcttcgccctgttgagatcgccctttagcgtttcgagacggccctga

SEQ ID NO: 321 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctcaacttcttcgtggtatctcgtttgnnngcaggtgcaatgacagcaagctacgtcaccctacattgcacctgcnntatgcaattcttctctgtgaatgtcggtttgaagagcgtttaaacgctcttcgcccggaagggacactaacctggtgtttcgagacggccctga

SEQ ID NO: 322 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctaccttgaatgaccagggtacctgcgtttgnnngcaggtgcaatggtcctctctgaagattgccgtcattgcacctgcnntatgcagccctttagctggtatctgcgtttgaagagcgtttaaacgctcttcgcccgattccttccaataacacaagtttcgagacggccctga

SEQ ID NO: 323 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgtgatggtgaatattcacagtttgnnngcaggtgcaatgttccccgatacgtactcgtaccattgcacctgcnntatgcatcacatggatggttactgaagtttgaagagcgtttaaacgctcttcgccccctatcatacgtatcctggtgtttcgagacggccctga

SEQ ID NO: 324 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctacatagacccctgttgtaaggtttgnnngcaggtgcaatgctacccgtcgcctaactaggacattgcacctgcnntatgcacatcggcctgacccccatgcgtttgaagagcgtttaaacgctcttcgcccttgggactcctcggttatttgtttcgagacggccctga

SEQ ID NO: 325 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctggagagccaacccctaccaggtttgnnngcaggtgcaatgtcccagactgtttcaccatgccattgcacctgcnntatgcagtatcctggtgggatttacagtttgaagagcgtttaaacgctcttcgccccagtgcaacttactattacggtttcgagacggccctga

SEQ ID NO: 326 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctttcatagacctaatgtgactgtttgnnngcaggtgcaatgcgcaacaactataggccccatcattgcacctgcnntatgcaagcacacaagcacgttacccgtttgaagagcgtttaaacgctcttcgcccacacaacactgatgaggtccgtttcgagacggccctga

SEQ ID NO: 327 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctactttccatgtcagccatacgtttgnnngcaggtgcaatggagagaaactaatcgcacgcgcattgcacctgcnntatgcagaggcggtcaggatggctgcgtttgaagagcgtttaaacgctcttcgcccggccatttatagtgttgaccgtttcgagacggccctga

SEQ ID NO: 328 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctagaagttggtgacgttctgcgtttgnnngcaggtgcaatgttacgattccgcatcacgctccattgcacctgcnntatgcacaggcacgatagatacaaaggtttgaagagcgtttaaacgctcttcgcccgctcaaaccattacagaagggtttcgagacggccctga

SEQ ID NO: 329 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctggccatttatagtgttgaccgtttgnnngcaggtgcaatgagcgaaacagcgtaaagcgtccattgcacctgcnntatgcaaatcccttgccccagcacatgtttgaagagcgtttaaacgctcttcgcccagcacacaagcacgttacccgtttcgagacggccctga

SEQ ID NO: 330 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctccctcaccttctacccagccgtttgnnngcaggtgcaatgaacaaccgagtatatcccgcccattgcacctgcnntatgcacaaagacctgcggcgagggcgtttgaagagcgtttaaacgctcttcgcccctgaaattagacatcaaagcgtttcgagacggccctga

SEQ ID NO: 331 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctacaggtgacccaaccttcaggtttgnnngcaggtgcaatgagaagccacttccaacgttcgcattgcacctgcnntatgcacaaagacctgcggcgagggcgtttgaagagcgtttaaacgctcttcgccctgttgagatcgccctttagcgtttcgagacggccctga

SEQ ID NO: 332 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcctatcatacgtatcctggtgtttgnnngcaggtgcaatgggcggcaaaaaaccctattcgcattgcacctgcnntatgcacaatacagggatacccactagtttgaagagcgtttaaacgctcttcgccccggctacactcgcggtcatcgtttcgagacggccctga

SEQ ID NO: 333 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctactttccatgtcagccatacgtttgnnngcaggtgcaatgtgctaactcagtgacctcacgcattgcacctgcnntatgcactgcagttgagctggcaatcgtttgaagagcgtttaaacgctcttcgcccgacttatatagcgcccgctagtttcgagacggccctga

SEQ ID NO: 334 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctgttacactctatctacacctgtttgnnngcaggtgcaatgcagcataccaccttatagccccattgcacctgcnntatgcatctttgaaccattgtgttatgtttgaagagcgtttaaacgctcttcgcccactatcacgtttagccccgcgtttcgagacggccctga

SEQ ID NO: 335 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctaccttgtcgatggcaatagccaaggtttgnnngcaggtgcaatgatgctctaccgacaggaccgtcattgcacctgcnntatgcactgcagttgagctggcaatcgtttgaagagcgtttaaacgctcttcgcccgtcgttatcacgtagcgcacgtttcgagacggccctga

SEQ ID NO: 336 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctactttccatgtcagccatacgtttgnnngcaggtgcaatgtacccatcgatatccagtccccattgcacctgcnntatgcatcacatggatggttactgaagtttgaagagcgtttaaacgctcttcgccctgaatggcggtcgatagctcgtttcgagacggccctga

SEQ ID NO: 337 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctacctatcatacgtatcctgggtttgnnngcaggtgcaatgcctccgactgtatccggtctacattgcacctgcnntatgcatctttgaaccattgtgttatgtttgaagagcgtttaaacgctcttcgccccgtcccacgcgtaacgcggtgtttcgagacggccctga

SEQ ID NO: 338 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctctggaaggtgaggcctaggcgtttgnnngcaggtgcaatgcgctaaggcaacccatatcggcattgcacctgcnntatgcacaaagacctgcggcgagggcgtttgaagagcgtttaaacgctcttcgcccatatacgcacgctagctacggtttcgagacggccctga

SEQ ID NO: 339 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctgattccttccaataacacaagtttgnnngcaggtgcaatggactattggcaacccacggaacattgcacctgcnntatgcagttacactctatctacacctgtttgaagagcgtttaaacgctcttcgccccacccggcgcgactcactaagtttcgagacggccctga

SEQ ID NO: 340 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctttgggactcctcggttatttgtttgnnngcaggtgcaatgcaaaactcgaccaccgtcaagcattgcacctgcnntatgcatgtcgatggcaatagccaaggtttgaagagcgtttaaacgctcttcgcccggccggacgaattacgataggtttcgagacggccctga

SEQ ID NO: 341 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctggaagggacactaacctggtgtttgnnngcaggtgcaatgtgagaaccacgagttcaagggcattgcacctgcnntatgcaagaagttggtgacgttctgcgtttgaagagcgtttaaacgctcttcgcccttcgcggcggagaagttagggtttcgagacggccctga

SEQ ID NO: 342 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctgtatcctggtgggatttacagtttgnnngcaggtgcaatgcctgtccttagcccttacatgcattgcacctgcnntatgcaactttccatgtcagccatacgtttgaagagcgtttaaacgctcttcgcccgtccccgcggtaacacgacggtttcgagacggccctga

SEQ ID NO: 343 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctaccttgcaagccttacctgtagcggtttgnnngcaggtgcaatgtgcatgaagtgacagcacgctcattgcacctgcnntatgcatgaatgaccagggtacctgcgtttgaagagcgtttaaacgctcttcgccctaatacgagcggttccggacgtttcgagacggccctga

SEQ ID NO: 344 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctcatcggcctgacccccatgcgtttgnnngcaggtgcaatgttaacggcccatcccctaacacattgcacctgcnntatgcactggaaggtgaggcctaggcgtttgaagagcgtttaaacgctcttcgcccagtcgtcgcgtgtttagtctgtttcgagacggccctga

SEQ ID NO: 345 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctaccttgttgggcagctgtatgatcgtttgnnngcaggtgcaatggaaccgacgaccaagtagaagcattgcacctgcnntatgcaagctgtagtagagtccccatgtttgaagagcgtttaaacgctcttcgccccgtgcgattttaagcgcgccgtttcgagacggccctga

SEQ ID NO: 346 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctaccttgcccacaaagtatggcggtgtttgnnngcaggtgcaatgcacactttccaaacgccggaccattgcacctgcnntatgcatgtcgatggcaatagccaaggtttgaagagcgtttaaacgctcttcgcccacgcgatatcatacaacgccgtttcgagacggccctga

SEQ ID NO: 347 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctcatcggcctgacccccatgcgtttgnnngcaggtgcaatgacacagcagctcaccaatcaccattgcacctgcnntatgcaagctgtagtagagtccccatgtttgaagagcgtttaaacgctcttcgccccttaaaaaccgtcattcgcggtttcgagacggccctga

SEQ ID NO: 348 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcctatcatacgtatcctggtgtttgnnngcaggtgcaatgctcccgactataagacacgcccattgcacctgcnntatgcaacaggtgacccaaccttcaggtttgaagagcgtttaaacgctcttcgccctagtaagccgtccgatgcgcgtttcgagacggccctga

SEQ ID NO: 349 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctacatagacccctgttgtaaggtttgnnngcaggtgcaatgagatgcccgatcagctcaacccattgcacctgcnntatgcatgtcgatggcaatagccaaggtttgaagagcgtttaaacgctcttcgcccgtatcggcccgtagtacgtagtttcgagacggccctga

SEQ ID NO: 350 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctcccaccaggatacgtatgatgtttgnnngcaggtgcaatggaccttattccgattagcccgcattgcacctgcnntatgcaatcagttctgagcaactcgtgtttgaagagcgtttaaacgctcttcgcccatacgttgttagtacgtccggtttcgagacggccctga

SEQ ID NO: 351 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctggagagccaacccctaccaggtttgnnngcaggtgcaatgaaatgcgcccttgacacgactcattgcacctgcnntatgcagttacactctatctacacctgtttgaagagcgtttaaacgctcttcgccccttctacggagtcaacgcgcgtttcgagacggccctga

SEQ ID NO: 352 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcagcttgtccaactggtcgggtttgnnngcaggtgcaatgcgatatgtcccctcttccctacattgcacctgcnntatgcacgcgtccactggattcataggtttgaagagcgtttaaacgctcttcgcccgttaaactaaatcggcgcgcgtttcgagacggccctga

SEQ ID NO: 353 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctaccttcacatggatggttactgaagtttgnnngcaggtgcaatgaatactcctcttccggcttcgcattgcacctgcnntatgcagttcgcgagcgtcctttagcgtttgaagagcgtttaaacgctcttcgccccgtgccggcgggtaacccctgtttcgagacggccctga

SEQ ID NO: 354 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctctgctagccaacaccactgagtttgnnngcaggtgcaatgccttgagcagcgcaccgttatcattgcacctgcnntatgcaccgctgaatccgtattgcgagtttgaagagcgtttaaacgctcttcgccctcgcgtgattagggattcgtgtttcgagacggccctga

SEQ ID NO: 355 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttctttgaaccattgtgttatgtttgnnngcaggtgcaatggaaagccacacagtcatcccgcattgcacctgcnntatgcaagtaatgcacccgtcgatccgtttgaagagcgtttaaacgctcttcgcccccgcggtcacggttggtacggtttcgagacggccctga

SEQ ID NO: 356 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctaccttctttgaaccattgtgttatgtttgnnngcaggtgcaatgcagcctaagcatacgatctcccattgcacctgcnntatgcaagtacgcgcgaacaagtattgtttgaagagcgtttaaacgctcttcgcccgccgcacgaagtatcctacggtttcgagacggccctga

SEQ ID NO: 357 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctgcagttgagctggcaatcgtttgnnngcaggtgcaatgttctacctccattcactggcgcattgcacctgcnntatgcatatgacgcgcgcccgcctcagtttgaagagcgtttaaacgctcttcgccctatagttgacggtcccgcgtgtttcgagacggccctga

SEQ ID NO: 358 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctcagcttgtccaactggtcgggtttgnnngcaggtgcaatgatcgactcgaaccagctaccacattgcacctgcnntatgcataagcggcaatcgccgtatagtttgaagagcgtttaaacgctcttcgcccgagcgttacggcacgacgccgtttcgagacggccctga

SEQ ID NO: 359 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctagctgtagtagagtccccatgtttgnnngcaggtgcaatgctcactatcacacgggactcacattgcacctgcnntatgcatactcgcgtaaacttcatacgtttgaagagcgtttaaacgctcttcgcccgtgtacgggcgtcatccgacgtttcgagacggccctga

SEQ ID NO: 360 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctcaatacagggatacccactagtttgnnngcaggtgcaatggctaaatgtaccccaacaccccattgcacctgcnntatgcactagacggcgcattagacgtgtttgaagagcgtttaaacgctcttcgccctttcatcgccgacgcgtcaggtttcgagacggccctga

SEQ ID NO: 361 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctggactgagagctgttgacacgtttgnnngcaggtgcaatgtcaatgccaacacacctctcgcattgcacctgcnntatgcatagaacccggatctcgcgaagtttgaagagcgtttaaacgctcttcgcccctttcgtcgcagcggtcatcgtttcgagacggccctga

SEQ ID NO: 362 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctggccatttatagtgttgaccgtttgnnngcaggtgcaatggcgcaccatgaataaagcaggcattgcacctgcnntatgcaacgaacttgatccgttccgtgtttgaagagcgtttaaacgctcttcgcccgacatcccgccgccgttgttgtttcgagacggccctga

SEQ ID NO: 363 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctctggaaggtgaggcctaggcgtttgnnngcaggtgcaatgcacaatccgccgtcaaatagccattgcacctgcnntatgcatcgccgtgccgtttcacgaggtttgaagagcgtttaaacgctcttcgcccgggcgcgcatgacgaccgaagtttcgagacggccctga

SEQ ID NO: 364 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctattcttctctgtgaatgtcggtttgnnngcaggtgcaatgcagaaactgaccaggcatacccattgcacctgcnntatgcattatcgcgtcgtgatcggcagtttgaagagcgtttaaacgctcttcgccctcgtacggtaacttatcgccgtttcgagacggccctga

SEQ ID NO: 365 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctcatcggcctgacccccatgcgtttgnnngcaggtgcaatgaccctgatctccaagactcgacattgcacctgcnntatgcagtcggtagacgacccggtgggtttgaagagcgtttaaacgctcttcgccctcgcgataggcggtatctgagtttcgagacggccctga

SEQ ID NO: 366 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctctgaaattagacatcaaagcgtttgnnngcaggtgcaatggctctgtaccgagtgctgctacattgcacctgcnntatgcagggcgtagttcgataacccagtttgaagagcgtttaaacgctcttcgccctatgatccctaccaagcgcggtttcgagacggccctga

SEQ ID NO: 367 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctacggggtcgaacctacgcgcgtttgnnngcaggtgcaatgcctcgagatgatcttcaccctcattgcacctgcnntatgcagccgtctagcgaaattgccggtttgaagagcgtttaaacgctcttcgcccgccgacagcgccggaaacgagtttcgagacggccctga

SEQ ID NO: 368 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctgcatcgaacgtcccgacgatgtttgnnngcaggtgcaatgcgcaaccaaaaaatagcccggcattgcacctgcnntatgcaatacgcgaatgcgttgacacgtttgaagagcgtttaaacgctcttcgcccgcactcgcgcgaatttattggtttcgagacggccctga

SEQ ID NO: 369 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctaatacgccgcgtttcgaagagtttgnnngcaggtgcaatgtacttgcccatccaagagctccattgcacctgcnntatgcatcgcggtgtggattgagtcggtttgaagagcgtttaaacgctcttcgcccatgcgaccatagtatcgccggtttcgagacggccctga

SEQ ID NO: 370 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctctacgacgccccgtgctatcgtttgnnngcaggtgcaatggaagcatccctcgaaaactcgcattgcacctgcnntatgcacgcgccttttcttacgataggtttgaagagcgtttaaacgctcttcgcccacgaaccggtctaccgcatagtttcgagacggccctga

SEQ ID NO: 371 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctttcttctaccgcgccgataggtttgnnngcaggtgcaatgcaaagtgcttcaacccgtccccattgcacctgcnntatgcatactcacagtacgtcgttcggtttgaagagcgtttaaacgctcttcgcccgcgtaaccgtagacccctcggtttcgagacggccctga

SEQ ID NO: 372 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctacctcgatacacgcgcgctagtttgnnngcaggtgcaatgatctctagcacctagcactggcattgcacctgcnntatgcagttcaagtaatggcgccgcggtttgaagagcgtttaaacgctcttcgcccgttcgtcaagtccgccgcaggtttcgagacggccctga

SEQ ID NO: 373 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctgttcactacgagtgtattccgtttgnnngcaggtgcaatgtcctactcatgagacatcgcgcattgcacctgcnntatgcacagcttcaggaccccgatccgtttgaagagcgtttaaacgctcttcgcccgctgacatcggctttgctgcgtttcgagacggccctga

SEQ ID NO: 374 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgcaggtccttaccttgttccgtttgnnngcaggtgcaatgctcatgtttcccaccatagcgcattgcacctgcnntatgcagtagaccaggcacccaacctgtttgaagagcgtttaaacgctcttcgccctaaccatagtacccagttgtgtttcgagacggccctga

SEQ ID NO: 375 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctacctaccaggctgacagttatcacgtttgnnngcaggtgcaatgcttcccaaagacaaggagtcccattgcacctgcnntatgcaggggcatgggccatgctgtagtttgaagagcgtttaaacgctcttcgcccgtggaagtctgtgtcgggaagtttcgagacggccctga

SEQ ID NO: 376 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctgcttgtggtagatagcaacggtttgnnngcaggtgcaatgtccgattcgcaccaagtcaagcattgcacctgcnntatgcagccatgggcaccttctccgcgtttgaagagcgtttaaacgctcttcgcccgtgtctgtatgacccacccggtttcgagacggccctga

SEQ ID NO: 377 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgtgcatacgtacttcgtccagtttgnnngcaggtgcaatgaccgacggataagaaaccctgcattgcacctgcnntatgcatcctgccgaggttgggtgccgtttgaagagcgtttaaacgctcttcgcccgcagtgaccgtcacgacctggtttcgagacggccctga

SEQ ID NO: 378 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctaccaggctgacagttatcacgtttgnnngcaggtgcaatgtctgattccctacaaccggaccattgcacctgcnntatgcataaccatagtacccagttgtgtttgaagagcgtttaaacgctcttcgcccgacccgaactgcgcgcgatagtttcgagacggccctga

SEQ ID NO: 379 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctttattcttgtccaggcaagggtttgnnngcaggtgcaatgagacattcagcacgagccgatcattgcacctgcnntatgcaccaagtaccttctccagcatgtttgaagagcgtttaaacgctcttcgcccccccgtacgacgcaagtcgtgtttcgagacggccctga

SEQ ID NO: 380 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgagaggtttccgaagactaagtttgnnngcaggtgcaatgccgtaaagatactagaggccccattgcacctgcnntatgcaatccccgtcgcagttgaactgtttgaagagcgtttaaacgctcttcgccccgcgcagtctaacgaagttcgtttcgagacggccctga

SEQ ID NO: 381 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctaccttcccacagttgtaccttatcgtttgnnngcaggtgcaatgcactgcactagatcaacgaggcattgcacctgcnntatgcaacctgtggtggttatggggcgtttgaagagcgtttaaacgctcttcgccccgcggtcgctagcgaaggaagtttcgagacggccctga

SEQ ID NO: 382 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctacctttcttctcagttcaagtaacgtttgnnngcaggtgcaatggccgacatctcacccaaagaacattgcacctgcnntatgcaccacagtactcacttggtccgtttgaagagcgtttaaacgctcttcgcccgcgattaatacgctccgaccgtttcgagacggccctga

SEQ ID NO: 383 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctaagcactacccctgccaaccgtttgnnngcaggtgcaatgtgatcccaactcctacgccatcattgcacctgcnntatgcagttgccaggtgagcccgctcgtttgaagagcgtttaaacgctcttcgccccttaccgtattaggcacgctgtttcgagacggccctga

SEQ ID NO: 384 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctaccttcctcagctgccatatgtgcgtttgnnngcaggtgcaatgagtacgagctataccgcacaccattgcacctgcnntatgcatgtgcttaagaccgggaccagtttgaagagcgtttaaacgctcttcgccctgcccgcggttcggtgttgagtttcgagacggccctga

SEQ ID NO: 385 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctctagcaagccataacaaaacgtttgnnngcaggtgcaatggcctttgaaatacctctcccccattgcacctgcnntatgcagagccgctgtgatcataccagtttgaagagcgtttaaacgctcttcgcccagttgcacccgcggttaacggtttcgagacggccctga

SEQ ID NO: 386 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttcctcagctgccatatgtgcgtttgnnngcaggtgcaatgcaaatggtagtcagccacacgcattgcacctgcnntatgcagcaggtccttaccttgttccgtttgaagagcgtttaaacgctcttcgcccatcgcgttgttgcatgacgggtttcgagacggccctga

SEQ ID NO: 387 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctaaagcagtcgcccctgcttggtttgnnngcaggtgcaatgctctgaccgaatcgtacgttccattgcacctgcnntatgcatcacacttggagttacagcagtttgaagagcgtttaaacgctcttcgcccccgtcgttcccttacggatagtttcgagacggccctga

SEQ ID NO: 388 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctaccttggcattggggacctcgggtgtttgnnngcaggtgcaatgatgacgcagtaacagccatcgcattgcacctgcnntatgcaatcccgtggcgatgtatcgagtttgaagagcgtttaaacgctcttcgcccgtccggaaacgtccgctaatgtttcgagacggccctga

SEQ ID NO: 389 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgagccgctgtgatcataccagtttgnnngcaggtgcaatgcagattgttatcaccgccaggcattgcacctgcnntatgcagagtaatgatgcgtcgccgagtttgaagagcgtttaaacgctcttcgccctcgatgcgtaactgcgcaccgtttcgagacggccctga

SEQ ID NO: 390 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctacctgtgtctgtatgacccacccggtttgnnngcaggtgcaatgaggaccccagacattgtttcgcattgcacctgcnntatgcacaccgtataatcggcacgttgtttgaagagcgtttaaacgctcttcgccctaggactgcacccgatcgtagtttcgagacggccctga

SEQ ID NO: 391 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctaaccccaacatcagcaagcggtttgnnngcaggtgcaatgccccggatctcaaccgtatatcattgcacctgcnntatgcatacgtcgcacgactacccatgtttgaagagcgtttaaacgctcttcgccccgcgacatatgaagagtcgagtttcgagacggccctga

SEQ ID NO: 392 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctaccttcccacagttgtaccttatcgtttgnnngcaggtgcaatggcaccctgaattgaacgacaccattgcacctgcnntatgcatatgctacgctcgtcgtgcggtttgaagagcgtttaaacgctcttcgcccaatctaacggataaccgggtgtttcgagacggccctga

SEQ ID NO: 393 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctgccatgggcaccttctccgcgtttgnnngcaggtgcaatggcatgaacaaacctgcaagcccattgcacctgcnntatgcaacgactgcgttttcgactgggtttgaagagcgtttaaacgctcttcgcccgcgttacgcgaaagtccgtagtttcgagacggccctga

SEQ ID NO: 394 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctaccttgtgaaaaggactgcaggccgtttgnnngcaggtgcaatgcacattcagaacctagccgagcattgcacctgcnntatgcactaacggccgaccgccccgcgtttgaagagcgtttaaacgctcttcgccccaacgaccggtacggatcatgtttcgagacggccctga

SEQ ID NO: 395 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctgtcacacctgagtaccactggtttgnnngcaggtgcaatgcgccgtcatactactcacatgcattgcacctgcnntatgcatgacgtaaaagacgcgtgtcgtttgaagagcgtttaaacgctcttcgcccaagcgcctcgggtagaccgagtttcgagacggccctga

SEQ ID NO: 396 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctacctccacagtactcacttggtccgtttgnnngcaggtgcaatgtcgcatcgccactataacgtgcattgcacctgcnntatgcacgtccgggtttagcattcgagtttgaagagcgtttaaacgctcttcgcccacgtagcgcgagaactccgcgtttcgagacggccctga

SEQ ID NO: 397 - DNA, 236 bp, artificial
  oligo
  misc_feature (93)..(95): n is a, c, g, or t
  misc_feature (141)..(142): n is a, c, g, or t
gaccgcgtctcacaccgcaagggcgtgaacagcgacggtttggagaccgtttaaacggtctctacctgcagtgaccgtcacgacctggtttgnnngcaggtgcaatgcctttaaccccgatcgaaccacattgcacctgcnntatgcatttcgcgatgcgcacgtattgtttgaagagcgtttaaacgctcttcgcccttgaaatcgattggtcgcgtgtttcgagacggccctga

SEQ ID NO: 398 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctattgtagtgcgtccccgccggtttgnnngcaggtgcaatgaccctattctgataccagccccattgcacctgcnntatgcaggatcgcggatatgtgcggtgtttgaagagcgtttaaacgctcttcgccccgattgtcaatcattacggcgtttcgagacggccctga

SEQ ID NO: 399 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctaccttgagtcgcgatatatgccgagtttgnnngcaggtgcaatgtgcaaccgacgcagccaaacacattgcacctgcnntatgcatgatgcgcacgatcggtggcgtttgaagagcgtttaaacgctcttcgccctaacgaggacgacgctcaacgtttcgagacggccctga

SEQ ID NO: 400 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctcgacttcgatttgtactccggtttgnnngcaggtgcaatggacggtccatcgcctatactgcattgcacctgcnntatgcatgcgcctaacatcgtaccgcgtttgaagagcgtttaaacgctcttcgccccttgcaacctgtaccgcgtcgtttcgagacggccctga

SEQ ID NO: 401 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaccaacgtcgattgaccggagtttgnnngcaggtgcaatgccccgatacgtatctacagtccattgcacctgcnntatgcataccggcctacggtcgctatgtttgaagagcgtttaaacgctcttcgcccgcgggaccccgttcggatacgtttcgagacggccctga

SEQ ID NO: 402 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttttcacacggttgtcgtcgcgtttgnnngcaggtgcaatgccagtatgactttctagcccccattgcacctgcnntatgcacgacgtcgctttagctcgttgtttgaagagcgtttaaacgctcttcgcccaccaccgtgaacgtccgacggtttcgagacggccctga

SEQ ID NO: 403 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctttgggggatactcacgggatgtttgnnngcaggtgcaatgccacacaaacgcctctcgatgcattgcacctgcnntatgcatacttgtacctgaaagctcggtttgaagagcgtttaaacgctcttcgccctcgacaacccctacatcgtggtttcgagacggccctga

SEQ ID NO: 404 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctaccttatccggttacctcatagtcgtttgnnngcaggtgcaatgcaaggaatccttacgcccacacattgcacctgcnntatgcagaagcgagagaatctcctcggtttgaagagcgtttaaacgctcttcgcccttattacgtgcgccggccgggtttcgagacggccctga

SEQ ID NO: 405 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctaagaggcttcttatgtcggcgtttgnnngcaggtgcaatgcctcgctacgtcacacaacgtcattgcacctgcnntatgcagaagcgagagaatctcctcggtttgaagagcgtttaaacgctcttcgcccgctataccggtcgcgacgaggtttcgagacggccctga

SEQ ID NO: 406 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctgtgacccgaggaaccggtgcgtttgnnngcaggtgcaatgttgagtactagcatacgcccccattgcacctgcnntatgcagaaccgcacgtggaggatgcgtttgaagagcgtttaaacgctcttcgcccgcagcacaagatcgcgcgtcgtttcgagacggccctga

SEQ ID NO: 407 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctagggcacgtcaactctacacgtttgnnngcaggtgcaatggagtacgccacgctaatcgagcattgcacctgcnntatgcaggggcacgatggcctttaccgtttgaagagcgtttaaacgctcttcgcccgacatatacgcgatacgacagtttcgagacggccctga

SEQ ID NO: 408 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctaccttcgacaacccctacatcgtggtttgnnngcaggtgcaatgcccgcaaatccgcgttaaaaccattgcacctgcnntatgcacgagcgtcgaaactcgacgcgtttgaagagcgtttaaacgctcttcgccccgctgccgcgttaggacatcgtttcgagacggccctga

SEQ ID NO: 409 - DNA, 236 bp, artificial
  oligo
  misc_feature (93)..(95): n is a, c, g, or t
  misc_feature (141)..(142): n is a, c, g, or t
gaccgcgtctcacaccggcatccacattcttacaggcgtttggagaccgtttaaacggtctctacctagggcacgtcaactctacacgtttgnnngcaggtgcaatgtgcagctattgacctcatcggcattgcacctgcnntatgcatcgctaacggttggaccgtggtttgaagagcgtttaaacgctcttcgccctacgacttctacacgccgtagtttcgagacggccctga

SEQ ID NO: 410 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctacctcattgtatacgccttcctgggtttgnnngcaggtgcaatgcctattgtaagagaacgcccccattgcacctgcnntatgcacggcgaaagcgccgattagagtttgaagagcgtttaaacgctcttcgccccggatgccgatccgttaaatgtttcgagacggccctga

SEQ ID NO: 411 - DNA, 236 bp, artificial
  oligo
  misc_feature (93)..(95): n is a, c, g, or t
  misc_feature (141)..(142): n is a, c, g, or t
gaccgcgtctcacaccggcgccagtgtttgcgctcccgtttggagaccgtttaaacggtctctacctagggcacgtcaactctacacgtttgnnngcaggtgcaatgcccatcatcggctttccacgacattgcacctgcnntatgcatcgtggcgtgcgaattcgacgtttgaagagcgtttaaacgctcttcgcccgaccggcgagtccgtaccatgtttcgagacggccctga

SEQ ID NO: 412 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctcaactttggctccgtgcgccgtttgnnngcaggtgcaatgccgccaaaaagccgaaagtctcattgcacctgcnntatgcatagcgcgttgtcatcccgcggtttgaagagcgtttaaacgctcttcgcccatccactcgcgacgcctaatgtttcgagacggccctga

SEQ ID NO: 413 - DNA, 236 bp, artificial
  oligo
  misc_feature (93)..(95): n is a, c, g, or t
  misc_feature (141)..(142): n is a, c, g, or t
gaccgcgtctcacaccgtacttgtacctgaaagctcggtttggagaccgtttaaacggtctctacctgaaccgcacgtggaggatgcgtttgnnngcaggtgcaatgccgacgcagattccgtactaccattgcacctgcnntatgcagggtgggtcgttactacgcggtttgaagagcgtttaaacgctcttcgcccgatcgctaggattgcgcggggtttcgagacggccctga

SEQ ID NO: 414 - DNA, 236 bp, artificial
  oligo
  misc_feature (93)..(95): n is a, c, g, or t
  misc_feature (141)..(142): n is a, c, g, or t
gaccgcgtctcacaccgctcacggcagcgcacgcacagtttggagaccgtttaaacggtctctacctctaacgtcgcgtcgagtcgggtttgnnngcaggtgcaatgcttctagaccgacctacatggcattgcacctgcnntatgcacccgtatcgccgagcaaactgtttgaagagcgtttaaacgctcttcgccccccggacggttcgtttgcgtgtttcgagacggccctga

SEQ ID NO: 415 - DNA, 236 bp, artificial
  oligo
  misc_feature (93)..(95): n is a, c, g, or t
  misc_feature (141)..(142): n is a, c, g, or t
gaccgcgtctcacaccgcaacggcacgtacgccatcggtttggagaccgtttaaacggtctctacctgacgggtaatgcgtgcgttcgtttgnnngcaggtgcaatgctcctaggtccaatcccatcccattgcacctgcnntatgcacgagtaataacgacgacttcgtttgaagagcgtttaaacgctcttcgcccactatgcggtaggtacgccggtttcgagacggccctga

SEQ ID NO: 416 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctagtcgtgcgatcgtggtgtggtttgnnngcaggtgcaatgcgtccccaataatataccggccattgcacctgcnntatgcaactcgcgctaatcgctaagagtttgaagagcgtttaaacgctcttcgccccggccgaacagcgtattatagtttcgagacggccctga

SEQ ID NO: 417 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctaccttatgcggcgcggttaaaagggtttgnnngcaggtgcaatgcgcagcctttacatgactcaccattgcacctgcnntatgcaccgcgttgcacttgaccggggtttgaagagcgtttaaacgctcttcgccccgtagccgcatcaaaatcgcgtttcgagacggccctga

SEQ ID NO: 418 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctaccttcacccataggagccacagcgtttgnnngcaggtgcaatgccgataaagaaccaccgacggcattgcacctgcnntatgcacagtgcaacttactattacggtttgaagagcgtttaaacgctcttcgccctctgggcatgtgggtccggcgtttcgagacggccctga

SEQ ID NO: 419 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctaccttgttgggcagctgtatgatcgtttgnnngcaggtgcaatgtgtccctatgaccgaatcgtgcattgcacctgcnntatgcactaaagggcgatctcaacaagtttgaagagcgtttaaacgctcttcgcccacctatcatacgtatcctgggtttcgagacggccctga

SEQ ID NO: 420 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctaccttgttgggcagctgtatgatcgtttgnnngcaggtgcaatgcgccatcttactaccaccccacattgcacctgcnntatgcacctatcatacgtatcctggtgtttgaagagcgtttaaacgctcttcgcccttcatagacctaatgtgactgtttcgagacggccctga

SEQ ID NO: 421 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctatcagttctgagcaactcgtgtttgnnngcaggtgcaatgagcaactagtaagcaccagggcattgcacctgcnntatgcaccctcaccttctacccagccgtttgaagagcgtttaaacgctcttcgcccggactgagagctgttgacacgtttcgagacggccctga

SEQ ID NO: 422 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctctgctagccaacaccactgagtttgnnngcaggtgcaatggaaacccgaagccatcgccatcattgcacctgcnntatgcacagcttgtccaactggtcgggtttgaagagcgtttaaacgctcttcgccctctttgaaccattgtgttatgtttcgagacggccctga

SEQ ID NO: 423 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctgattccttccaataacacaagtttgnnngcaggtgcaatgtggagccaacgatctagaacccattgcacctgcnntatgcacgtcaccaagccattgtaccgtttgaagagcgtttaaacgctcttcgccccagcttgtccaactggtcgggtttcgagacggccctga

SEQ ID NO: 424 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctctggaaggtgaggcctaggcgtttgnnngcaggtgcaatgtcccgtcaccatattgacagccattgcacctgcnntatgcatgttgagatcgccctttagcgtttgaagagcgtttaaacgctcttcgcccggaagggacactaacctggtgtttcgagacggccctga

SEQ ID NO: 425 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctaccttctttgaaccattgtgttatgtttgnnngcaggtgcaatgacaactcccgtcttaaacccccattgcacctgcnntatgcatcacccataggagccacagcgtttgaagagcgtttaaacgctcttcgccctgtgatggtgaatattcacagtttcgagacggccctga

SEQ ID NO: 426 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctctaaagggcgatctcaacaagtttgnnngcaggtgcaatggatcagttgcgggactaaagccattgcacctgcnntatgcactccacaaagacctgcggcggtttgaagagcgtttaaacgctcttcgcccctggaaggtgaggcctaggcgtttcgagacggccctga

SEQ ID NO: 427 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctgaggcggtcaggatggctgcgtttgnnngcaggtgcaatgcaagaagcctcaaggaagccccattgcacctgcnntatgcattcatagacctaatgtgactgtttgaagagcgtttaaacgctcttcgccctgtgatggtgaatattcacagtttcgagacggccctga

SEQ ID NO: 428 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctacaggtgacccaaccttcaggtttgnnngcaggtgcaatggcaccaagagcttcactactccattgcacctgcnntatgcagaggcggtcaggatggctgcgtttgaagagcgtttaaacgctcttcgccctctgggcatgtgggtccggcgtttcgagacggccctga

SEQ ID NO: 429 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctcaacttcttcgtggtatctcgtttgnnngcaggtgcaatgcgacgcctattccgaaagtgacattgcacctgcnntatgcacccaccaggatacgtatgatgtttgaagagcgtttaaacgctcttcgcccacacaacactgatgaggtccgtttcgagacggccctga

SEQ ID NO: 430 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctctgaaattagacatcaaagcgtttgnnngcaggtgcaatgcccgcactaatctacgctaaccattgcacctgcnntatgcatgaatgaccagggtacctgcgtttgaagagcgtttaaacgctcttcgccccgtcaccaagccattgtaccgtttcgagacggccctga

SEQ ID NO: 431 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctctgcagttgagctggcaatcgtttgnnngcaggtgcaatgccccgatctgtttcaacgtagcattgcacctgcnntatgcaagctgtagtagagtccccatgtttgaagagcgtttaaacgctcttcgcccggactgagagctgttgacacgtttcgagacggccctga

SEQ ID NO: 432 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctaatcccttgccccagcacatgtttgnnngcaggtgcaatgtgaagtgtaccacaagctcgccattgcacctgcnntatgcaactttccatgtcagccatacgtttgaagagcgtttaaacgctcttcgccccaaagacctgcggcgagggcgtttcgagacggccctga

SEQ ID NO: 433 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctacctatcatacgtatcctgggtttgnnngcaggtgcaatgtatgtgccaacagctaccgtccattgcacctgcnntatgcatgaatgaccagggtacctgcgtttgaagagcgtttaaacgctcttcgcccatagacgcgtgacaaccgaagtttcgagacggccctga

SEQ ID NO: 434 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctaccttctgggcatgtgggtccggcgtttgnnngcaggtgcaatgggaaactccgccaatttccctcattgcacctgcnntatgcatcatcctgcatgctagaccagtttgaagagcgtttaaacgctcttcgcccccgcgagtcctggacataaggtttcgagacggccctga

SEQ ID NO: 435 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctctgcagttgagctggcaatcgtttgnnngcaggtgcaatgccctatcccaacgtcccataacattgcacctgcnntatgcatctttgaaccattgtgttatgtttgaagagcgtttaaacgctcttcgcccgcggcgctgtcgagatacacgtttcgagacggccctga

SEQ ID NO: 436 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctaccttctgggcatgtgggtccggcgtttgnnngcaggtgcaatgtcccgacctcgatcttactcccattgcacctgcnntatgcatcacatggatggttactgaagtttgaagagcgtttaaacgctcttcgcccttcgcatcgatccgctgtaagtttcgagacggccctga

SEQ ID NO: 437 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctactttccatgtcagccatacgtttgnnngcaggtgcaatgttatccgcccagagccaaactcattgcacctgcnntatgcattcatagacctaatgtgactgtttgaagagcgtttaaacgctcttcgccccacgattcacgcgtaggcgcgtttcgagacggccctga

SEQ ID NO: 438 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctaccttgttgagatcgccctttagcgtttgnnngcaggtgcaatggagaaccagctacaccccgaacattgcacctgcnntatgcattcatagacctaatgtgactgtttgaagagcgtttaaacgctcttcgcccattacgcgtacgacgggccagtttcgagacggccctga

SEQ ID NO: 439 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctacctggccatttatagtgttgaccgtttgnnngcaggtgcaatgatgcgctagactcgaattccccattgcacctgcnntatgcacaaagacctgcggcgagggcgtttgaagagcgtttaaacgctcttcgcccggggcggaccaaaattcgcggtttcgagacggccctga

SEQ ID NO: 440 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctttgggactcctcggttatttgtttgnnngcaggtgcaatgagccactacgctaaccacatgcattgcacctgcnntatgcagccctttagctggtatctgcgtttgaagagcgtttaaacgctcttcgcccgggaccgatccattaccgtagtttcgagacggccctga

SEQ ID NO: 441 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcgtcaccaagccattgtaccgtttgnnngcaggtgcaatgcgcctaagaactcccactgaccattgcacctgcnntatgcaacaggtgacccaaccttcaggtttgaagagcgtttaaacgctcttcgccccgaacccgtcgcccgcttacgtttcgagacggccctga

SEQ ID NO: 442 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttgtcgatggcaatagccaaggtttgnnngcaggtgcaatgccgaccctaaataccttgtggcattgcacctgcnntatgcagttacactctatctacacctgtttgaagagcgtttaaacgctcttcgccccgaccgtgttgttccgtcgggtttcgagacggccctga

SEQ ID NO: 443 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctcctatcatacgtatcctggtgtttgnnngcaggtgcaatgccccgtagataaacgagcaaccattgcacctgcnntatgcactgcagttgagctggcaatcgtttgaagagcgtttaaacgctcttcgcccgtcgcgacgctaattccctagtttcgagacggccctga

SEQ ID NO: 444 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgaatgaccagggtacctgcgtttgnnngcaggtgcaatggcccacataccaatagttccccattgcacctgcnntatgcatcacccataggagccacagcgtttgaagagcgtttaaacgctcttcgccccaagtagcgcgcttagacatgtttcgagacggccctga

SEQ ID NO: 445 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctagatcatacagctgcccaacgtttgnnngcaggtgcaatgccaatcgatcccgaatgtacgcattgcacctgcnntatgcagtatcctggtgggatttacagtttgaagagcgtttaaacgctcttcgccccgtggcccgtttagcgtgtcgtttcgagacggccctga

SEQ ID NO: 446 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctgctcaaaccattacagaagggtttgnnngcaggtgcaatggacttaccatgacctaggcgccattgcacctgcnntatgcatgcccacaaagtatggcggtgtttgaagagcgtttaaacgctcttcgcccagttgcttcgttcgaccagtgtttcgagacggccctga

SEQ ID NO: 447 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctacatagacccctgttgtaaggtttgnnngcaggtgcaatgctcgatccgcacatgcataaccattgcacctgcnntatgcacagcttgtccaactggtcgggtttgaagagcgtttaaacgctcttcgcccgggcgtatcggtcaagacgagtttcgagacggccctga

SEQ ID NO: 448 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctgaggcggtcaggatggctgcgtttgnnngcaggtgcaatgtgcatactcccactgaatccccattgcacctgcnntatgcaagatcatacagctgcccaacgtttgaagagcgtttaaacgctcttcgccctaatgctccgcggccgcttagtttcgagacggccctga

SEQ ID NO: 449 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctgtatcctggtgggatttacagtttgnnngcaggtgcaatgcgcccaaattcgacggaatcccattgcacctgcnntatgcatgtgatggtgaatattcacagtttgaagagcgtttaaacgctcttcgcccttcaccgcgcaaacgatggcgtttcgagacggccctga

SEQ ID NO: 450 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctcaacttcttcgtggtatctcgtttgnnngcaggtgcaatgccgcaacacagtataatccggcattgcacctgcnntatgcacaggcacgatagatacaaaggtttgaagagcgtttaaacgctcttcgcccgacgagtcgtcgcgctactggtttcgagacggccctga

SEQ ID NO: 451 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctatcagttctgagcaactcgtgtttgnnngcaggtgcaatgcccttgataacgaccgaagcgcattgcacctgcnntatgcacctatcatacgtatcctggtgtttgaagagcgtttaaacgctcttcgcccggccggtatacgtctacgaggtttcgagacggccctga

SEQ ID NO: 452 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctctccacaaagacctgcggcggtttgnnngcaggtgcaatggattccatcccgagcattacccattgcacctgcnntatgcattgttacagccttattacacgtttgaagagcgtttaaacgctcttcgccctcggccgtataggaatggtagtttcgagacggccctga

SEQ ID NO: 453 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctggaagggacactaacctggtgtttgnnngcaggtgcaatgctttcgcgccaagagacaacgcattgcacctgcnntatgcacaatcacgcgccgcgagactgtttgaagagcgtttaaacgctcttcgcccccggaatcgttaagttacgtgtttcgagacggccctga

SEQ ID NO: 454 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctggccatttatagtgttgaccgtttgnnngcaggtgcaatgtgccccagtcagacttccacacattgcacctgcnntatgcacgtatatcccggcggcatcggtttgaagagcgtttaaacgctcttcgcccagcgttaaccgcgtggcgtggtttcgagacggccctga

SEQ ID NO: 455 - DNA, 236 bp, artificial
  oligo
  misc_feature (93)..(95): n is a, c, g, or t
  misc_feature (141)..(142): n is a, c, g, or t
gaccgcgtctcacaccggattccttccaataacacaagtttggagaccgtttaaacggtctctacctacaggtgacccaaccttcaggtttgnnngcaggtgcaatggtccagaacgactagcaagaccattgcacctgcnntatgcacgcacattagtggtgcgcgcgtttgaagagcgtttaaacgctcttcgcccaccggcggaactattcgacggtttcgagacggccctga

SEQ ID NO: 456 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgggactcctcggttatttagtttgnnngcaggtgcaatggcgcctaaagcagcacaatcccattgcacctgcnntatgcaactgcgcgtataacgatagggtttgaagagcgtttaaacgctcttcgcccttacagcttacgtcggcacggtttcgagacggccctga

SEQ ID NO: 457 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctagaagttggtgacgttctgcgtttgnnngcaggtgcaatgtcccagatgcgcaaaaagcaccattgcacctgcnntatgcaaccgaccaaattccccggcggtttgaagagcgtttaaacgctcttcgcccactgattcgaactcgccgtggtttcgagacggccctga

SEQ ID NO: 458 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttctgggcatgtgggtccggcgtttgnnngcaggtgcaatgatccaccgtactccttagacccattgcacctgcnntatgcaaagggtagcgtaattcgtcggtttgaagagcgtttaaacgctcttcgcccataggggcggcgattccgacgtttcgagacggccctga

SEQ ID NO: 459 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctaccttgcccacaaagtatggcggtgtttgnnngcaggtgcaatgtagcagaccatccggccgaaacattgcacctgcnntatgcacggcgaaacgtaagcacgccgtttgaagagcgtttaaacgctcttcgccccggattgcgaaactatccgggtttcgagacggccctga

SEQ ID NO: 460 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctctgaaattagacatcaaagcgtttgnnngcaggtgcaatgagctgaacttcaccgacgacgcattgcacctgcnntatgcagccgaattgcgacacgaggtgtttgaagagcgtttaaacgctcttcgcccaacggcggacgacctacaatgtttcgagacggccctga

SEQ ID NO: 461 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctctccacaaagacctgcggcggtttgnnngcaggtgcaatgcctatatggacactcagctcgcattgcacctgcnntatgcatacactacgggaactcgccggtttgaagagcgtttaaacgctcttcgcccgaacgtctaccgtcggcggagtttcgagacggccctga

SEQ ID NO: 462 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctgctagccaacaccactgagtttgnnngcaggtgcaatgcttagtacctccaacaaccggcattgcacctgcnntatgcatctacggccgcggttacgttgtttgaagagcgtttaaacgctcttcgccccacatctcgtcggacgctcggtttcgagacggccctga

SEQ ID NO: 463 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctatcagttctgagcaactcgtgtttgnnngcaggtgcaatgtcttctctccacggtgtgctacattgcacctgcnntatgcaatatgctagcgtcgcattcggtttgaagagcgtttaaacgctcttcgccctccgcaagttgcgtacgacggtttcgagacggccctga

SEQ ID NO: 464 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctcaggcacgatagatacaaaggtttgnnngcaggtgcaatgcaccacacaggtcgtttacagcattgcacctgcnntatgcacgacgttgagcgttagactggtttgaagagcgtttaaacgctcttcgccccagtatcgggtgcataacgcgtttcgagacggccctga

SEQ ID NO: 465 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctttgttacagccttattacacgtttgnnngcaggtgcaatgcccgtacctatagcactaggccattgcacctgcnntatgcatatagcgagcgacggacgccgtttgaagagcgtttaaacgctcttcgccccgtctgctcgaagtcgagttgtttcgagacggccctga

SEQ ID NO: 466 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctggactgagagctgttgacacgtttgnnngcaggtgcaatgctcatgcaattagacacgcggcattgcacctgcnntatgcatcgctctaatccggatgccggtttgaagagcgtttaaacgctcttcgcccaatcgtatttggtcgcgcgagtttcgagacggccctga

SEQ ID NO: 467 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctcgtcaccaagccattgtaccgtttgnnngcaggtgcaatgccccatatgctcgacagaggacattgcacctgcnntatgcactcgatatcaaccccgtaacgtttgaagagcgtttaaacgctcttcgcccactcgattgcgagcgtacccgtttcgagacggccctga

SEQ ID NO: 468 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttcgtttggccagacgcgtcagtttgnnngcaggtgcaatgggaagagtgcgtcatactccacattgcacctgcnntatgcacttacgatattagcgtcgaagtttgaagagcgtttaaacgctcttcgcccgcctcatcgcgacatcgtaggtttcgagacggccctga

SEQ ID NO: 469 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctaattattagattcgcgcgtcgtttgnnngcaggtgcaatgaactcagcgccgcaacataagcattgcacctgcnntatgcatcgccacctaattcgacggtgtttgaagagcgtttaaacgctcttcgcccgtgttcgacgcctctccggagtttcgagacggccctga

SEQ ID NO: 470 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctcgcgctagtccgacaccaaagtttgnnngcaggtgcaatgaccacatccgcttacactctgcattgcacctgcnntatgcactcgatcgtgcggtatacgtgtttgaagagcgtttaaacgctcttcgccccgcgcctatatgcggattccgtttcgagacggccctga

SEQ ID NO: 471 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgatcacgaatttatcgccgtgtttgnnngcaggtgcaatgagccaccgacctagtcttagccattgcacctgcnntatgcaaatccgctcgtgtgcgctatgtttgaagagcgtttaaacgctcttcgccccgttacgcctccgccgttccgtttcgagacggccctga

SEQ ID NO: 472 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttcgcacccgttattgaagcggtttgnnngcaggtgcaatgttcccacttacacgagatcggcattgcacctgcnntatgcagcgatacccggtgttcgattgtttgaagagcgtttaaacgctcttcgccctcgcgcatcgaaaagtaactgtttcgagacggccctga

SEQ ID NO: 473 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctattcgcgttttagccactcggtttgnnngcaggtgcaatggctctacgaatacactctgcgcattgcacctgcnntatgcatgttgcgacctttcgccgtagtttgaagagcgtttaaacgctcttcgccccgcccgaagtcggttagtctgtttcgagacggccctga

SEQ ID NO: 474 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctgccatgggcaccttctccgcgtttgnnngcaggtgcaatgaaaccgccctaacttatgccccattgcacctgcnntatgcagttgccaggtgagcccgctcgtttgaagagcgtttaaacgctcttcgcccgctgacatcggctttgctgcgtttcgagacggccctga

SEQ ID NO: 475 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctgagccgctgtgatcataccagtttgnnngcaggtgcaatggtaagccaagcagacagatggcattgcacctgcnntatgcagttgccaggtgagcccgctcgtttgaagagcgtttaaacgctcttcgcccgcaaagccgatgtcagcgccgtttcgagacggccctga

SEQ ID NO: 476 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctgttgccaggtgagcccgctcgtttgnnngcaggtgcaatggtcagagctcacgcatttacccattgcacctgcnntatgcagtgcatacgtacttcgtccagtttgaagagcgtttaaacgctcttcgcccaacatgactccccggaggccgtttcgagacggccctga

SEQ ID NO: 477 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctaccttggcattggggacctcgggtgtttgnnngcaggtgcaatgacgcagctacttcacacatcccattgcacctgcnntatgcagtggaagtctgtgtcgggaagtttgaagagcgtttaaacgctcttcgccctcctcagctgccatatgtgcgtttcgagacggccctga

SEQ ID NO: 478 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctcaagggcgtgaacagcgacggtttgnnngcaggtgcaatggtccgaagccttcaacagatccattgcacctgcnntatgcaaccaggcacccaacctcggcgtttgaagagcgtttaaacgctcttcgcccgagaagcactatgggggcatgtttcgagacggccctga

SEQ ID NO: 479 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgtgtctgtatgacccacccggtttgnnngcaggtgcaatgcctagttcgtcagatacccagcattgcacctgcnntatgcatgtgcttaagaccgggaccagtttgaagagcgtttaaacgctcttcgccctcgtggcgggtcgaattcgcgtttcgagacggccctga

SEQ ID NO: 480 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctgttgccaggtgagcccgctcgtttgnnngcaggtgcaatgtgccattccggtccagtatagcattgcacctgcnntatgcagcaggtccttaccttgttccgtttgaagagcgtttaaacgctcttcgcccgtgcgcgcgtagcgatcgacgtttcgagacggccctga

SEQ ID NO: 481 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctaccaggctgacagttatcacgtttgnnngcaggtgcaatgcaacccgaactcgaaatgacccattgcacctgcnntatgcatggcattggggacctcgggtgtttgaagagcgtttaaacgctcttcgccctcgaaatcgacacgtcgcgagtttcgagacggccctga

SEQ ID NO: 482 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgttgccaggtgagcccgctcgtttgnnngcaggtgcaatggccctcttcaggaagaatgagcattgcacctgcnntatgcagtagaccaggcacccaacctgtttgaagagcgtttaaacgctcttcgcccgttcgccgaataggtcgctagtttcgagacggccctga

SEQ ID NO: 483 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctaccttgctgtcgtgagtgccagccgtttgnnngcaggtgcaatgccaccaacgtaggtagaaacccattgcacctgcnntatgcaagccacgcttgctgatgttggtttgaagagcgtttaaacgctcttcgcccgtgtcgtagtacgtcgacaagtttcgagacggccctga

SEQ ID NO: 484 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgagaggtttccgaagactaagtttgnnngcaggtgcaatgccacggttacgcacaatcctgcattgcacctgcnntatgcagcttgtggtagatagcaacggtttgaagagcgtttaaacgctcttcgcccagcgtgttgcgtccgccgtagtttcgagacggccctga

SEQ ID NO: 485 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctacctccaagtaccttctccagcatgtttgnnngcaggtgcaatgagacttgcactttagccccaccattgcacctgcnntatgcagtagaccaggcacccaacctgtttgaagagcgtttaaacgctcttcgccctgttacgccacgctatttcggtttcgagacggccctga

SEQ ID NO: 486 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctctagcaagccataacaaaacgtttgnnngcaggtgcaatgctaccacgaccgaagcttagacattgcacctgcnntatgcagtagaccaggcacccaacctgtttgaagagcgtttaaacgctcttcgccccatcgtgaatcgatccgaaggtttcgagacggccctga

SEQ ID NO: 487 - DNA, 236 bp, artificial
  oligo
  misc_feature (93)..(95): n is a, c, g, or t
  misc_feature (141)..(142): n is a, c, g, or t
gaccgcgtctcacaccgatccccgtcgcagttgaactgtttggagaccgtttaaacggtctctacctaagcactacccctgccaaccgtttgnnngcaggtgcaatgtcacgcaaccctactggcaaacattgcacctgcnntatgcagtgcatacgtacttcgtccagtttgaagagcgtttaaacgctcttcgcccagataatatgcgccgctcgggtttcgagacggccctga

SEQ ID NO: 488 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctttcttctcagttcaagtaacgtttgnnngcaggtgcaatgttccgactaccggcatacactcattgcacctgcnntatgcatcctgccgaggttgggtgccgtttgaagagcgtttaaacgctcttcgccctacggtcacacgttgcgcctgtttcgagacggccctga

SEQ ID NO: 489 - DNA, 236 bp, artificial
  oligo
  misc_feature (93)..(95): n is a, c, g, or t
  misc_feature (141)..(142): n is a, c, g, or t
gaccgcgtctcacaccgaaccccaacatcagcaagcggtttggagaccgtttaaacggtctctacctgtgcatacgtacttcgtccagtttgnnngcaggtgcaatgccgcactacacgcgtttaatgcattgcacctgcnntatgcacgcgacatgcatcgctttcggtttgaagagcgtttaaacgctcttcgccccccccgtcggacgcgtatgagtttcgagacggccctga

SEQ ID NO: 490 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctaccttcccacagttgtaccttatcgtttgnnngcaggtgcaatgtttcaatccttcatccgccgccattgcacctgcnntatgcacaatcgcacgcgtaaccgtagtttgaagagcgtttaaacgctcttcgcccgcgagtcgagcaatttggcggtttcgagacggccctga

SEQ ID NO: 491 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctaccttggcattggggacctcgggtgtttgnnngcaggtgcaatgcgcaactaccctcatcctgaccattgcacctgcnntatgcatgtgcgccgtcgcaatagtagtttgaagagcgtttaaacgctcttcgccctcgtgaaagcgtaccgcactgtttcgagacggccctga

SEQ ID NO: 492 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctgtggaagtctgtgtcgggaagtttgnnngcaggtgcaatgtaccatccgtgaacaaccagccattgcacctgcnntatgcaacgttagtgtcgcaccgacggtttgaagagcgtttaaacgctcttcgcccctaatcggcgtcttcgcggcgtttcgagacggccctga

SEQ ID NO: 493 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctaccttcacacttggagttacagcagtttgnnngcaggtgcaatgcccctttattccacgttctgccattgcacctgcnntatgcaccgcgaccggacgtttatccgtttgaagagcgtttaaacgctcttcgcccgcgtcacttcgtcgaatcccgtttcgagacggccctga

SEQ ID NO: 494 - DNA, 236 bp, artificial
  oligo
  misc_feature (93)..(95): n is a, c, g, or t
  misc_feature (141)..(142): n is a, c, g, or t
gaccgcgtctcacaccggtcacacctgagtaccactggtttggagaccgtttaaacggtctctacctgcaaagccgatgtcagcgccgtttgnnngcaggtgcaatggaaacgacgatcgtcaaaggccattgcacctgcnntatgcatatgggctcgtcatcgttcggtttgaagagcgtttaaacgctcttcgcccaagcgttgttaatcgccgtagtttcgagacggccctga

SEQ ID NO: 495 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctgtggaagtctgtgtcgggaagtttgnnngcaggtgcaatgcagacctcgagacacacacaacattgcacctgcnntatgcagtaacaacgaaccggcccgggtttgaagagcgtttaaacgctcttcgcccgtcgacacgttagtagggcggtttcgagacggccctga

SEQ ID NO: 496 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctaccttcccacagttgtaccttatcgtttgnnngcaggtgcaatgctctgcttccacactgatcctcattgcacctgcnntatgcacgtgttaggtcgccgagcttgtttgaagagcgtttaaacgctcttcgcccacgtctattcgggcgtcgacgtttcgagacggccctga

SEQ ID NO: 497 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctaccaggcacccaacctcggcgtttgnnngcaggtgcaatggagatcggcaagaccaaacgacattgcacctgcnntatgcagtcccgtgacaaaccgtcgtgtttgaagagcgtttaaacgctcttcgccccgcgtattaccggtcttatggtttcgagacggccctga

SEQ ID NO: 498 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgtcacacctgagtaccactggtttgnnngcaggtgcaatgcccaacgctcgataagctcaccattgcacctgcnntatgcaacatcgcgtcggtctagtgagtttgaagagcgtttaaacgctcttcgccctgattcgacgggtagtcgcagtttcgagacggccctga

SEQ ID NO: 499 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctcggtagttgcgtagtagacggtttgnnngcaggtgcaatgggaattaactgcatcctcgcccattgcacctgcnntatgcatacggcgttttgaccccgtagtttgaagagcgtttaaacgctcttcgccccgttagttatgcgaacgcatgtttcgagacggccctga

SEQ ID NO: 500 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctcgggaagcctattcgccgcggtttgnnngcaggtgcaatggtactccactatgcacagaggcattgcacctgcnntatgcactaatcgtacgtatcgagtcgtttgaagagcgtttaaacgctcttcgccccactcgtcgctcgacaagatgtttcgagacggccctga

SEQ ID NO: 501 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctcgctaaggacgctcccgcatgtttgnnngcaggtgcaatgcacgatctagccctgaacaagcattgcacctgcnntatgcagcgagtcgctccgtcatagggtttgaagagcgtttaaacgctcttcgcccggtcgaggggcgcgcatttcgtttcgagacggccctga

SEQ ID NO: 502 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctccgttgacctttcggcgtcggtttgnnngcaggtgcaatgctcgtattccgaccctcgtttcattgcacctgcnntatgcaacgagtctggacgctcgacggtttgaagagcgtttaaacgctcttcgcccttgccgaagcgcggtagtatgtttcgagacggccctga

SEQ ID NO: 503 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctcggggtacgcggtcgcgaatgtttgnnngcaggtgcaatggaacgcaccactagctttctgcattgcacctgcnntatgcagacccgcgagattccgtattgtttgaagagcgtttaaacgctcttcgccccgcgcgccgagggctgtattgtttcgagacggccctga

SEQ ID NO: 504 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctctcctgggaaccgcacgtgggtttgnnngcaggtgcaatgtctaggtaacccacgcatcgtcattgcacctgcnntatgcatcgggggacggttatttctggtttgaagagcgtttaaacgctcttcgcccgcgccagtgtttgcgctcccgtttcgagacggccctga

SEQ ID NO: 505 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctaccttatccggttacctcatagtcgtttgnnngcaggtgcaatgagcaaaagcgaaaccccggtgcattgcacctgcnntatgcacggccatgcctgccagcttcgtttgaagagcgtttaaacgctcttcgcccccggctgtcgacgccagtacgtttcgagacggccctga

SEQ ID NO: 506 - DNA, 236 bp, artificial
  oligo
  misc_feature (93)..(95): n is a, c, g, or t
  misc_feature (141)..(142): n is a, c, g, or t
gaccgcgtctcacaccgcaactttggctccgtgcgccgtttggagaccgtttaaacggtctctacctctcctgggaaccgcacgtgggtttgnnngcaggtgcaatgcccaatcttcgtcaaacgggtcattgcacctgcnntatgcagtgcgctgccgtgagttgccgtttgaagagcgtttaaacgctcttcgccctagcgaaatcgtacccgagcgtttcgagacggccctga

SEQ ID NO: 507 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctagggcacgtcaactctacacgtttgnnngcaggtgcaatgcgagcttctccctaagatcagcattgcacctgcnntatgcagccttgagcccggtggggctgtttgaagagcgtttaaacgctcttcgcccccttacgctgcgtccgcgttgtttcgagacggccctga

SEQ ID NO: 508 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctatcctccacgtgcggttcccgtttgnnngcaggtgcaatgtgcgcagacaagaggtatgcacattgcacctgcnntatgcattctcggaagtcgaggacaggtttgaagagcgtttaaacgctcttcgccccgtataagtcctaagcgacggtttcgagacggccctga

SEQ ID NO: 509 - DNA, 236 bp, artificial
  oligo
  misc_feature (93)..(95): n is a, c, g, or t
  misc_feature (141)..(142): n is a, c, g, or t
gaccgcgtctcacaccggcgccagtgtttgcgctcccgtttggagaccgtttaaacggtctctacctctcgtagcttgccacactgtgtttgnnngcaggtgcaatgcccattgaacgactcatgcgtcattgcacctgcnntatgcaccggatccgttatctccggagtttgaagagcgtttaaacgctcttcgcccactccttcgctcgaattgcggtttcgagacggccctga

SEQ ID NO: 510 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctttctcggaagtcgaggacaggtttgnnngcaggtgcaatgtgtcccgaagatacacccctgcattgcacctgcnntatgcatcaacgtagcgctattatcggtttgaagagcgtttaaacgctcttcgccccttggcgcgattcgggttgcgtttcgagacggccctga

SEQ ID NO: 511 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctacctgaaccgcacgtggaggatgcgtttgnnngcaggtgcaatgcctccaggtttaacacagcctcattgcacctgcnntatgcacccgaaagggcgaatcgcgggtttgaagagcgtttaaacgctcttcgccctgcgtcgcgacccccatatcgtttcgagacggccctga

SEQ ID NO: 512 - DNA, 236 bp, artificial
  oligo
  misc_feature (93)..(95): n is a, c, g, or t
  misc_feature (141)..(142): n is a, c, g, or t
gaccgcgtctcacaccggctaccagcaccggttcctcgtttggagaccgtttaaacggtctctacctcgcccagggagcgcaaacacgtttgnnngcaggtgcaatggcaaccgccaacctgattcatcattgcacctgcnntatgcacgtcggacgttacgtaacttgtttgaagagcgtttaaacgctcttcgccctcgtatgacaatcgcgaaacgtttcgagacggccctga

SEQ ID NO: 513 - DNA, 236 bp, artificial
  oligo
  misc_feature (93)..(95): n is a, c, g, or t
  misc_feature (141)..(142): n is a, c, g, or t
gaccgcgtctcacaccgcggccatgcctgccagcttcgtttggagaccgtttaaacggtctctacctgggccagggtctgcatatgcgtttgnnngcaggtgcaatgccactaggcctcatcaattcgcattgcacctgcnntatgcaagggtatcgaccgtcgccctgtttgaagagcgtttaaacgctcttcgcccactaacgacgtcgactagccgtttcgagacggccctga

SEQ ID NO: 514 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctggccagaatacaggtcccgcgtttgnnngcaggtgcaatgagggactcagcaacgacaccacattgcacctgcnntatgcacgaccgaatctcgtgaacgggtttgaagagcgtttaaacgctcttcgccctcttcgacgctcggctaaacgtttcgagacggccctga

SEQ ID NO: 515 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctacttatcgttcgcgacgtaagtttgnnngcaggtgcaatgtgttcttatatcccagcgccccattgcacctgcnntatgcagtcacccggcggacttaagagtttgaagagcgtttaaacgctcttcgccccgccgatgacgtatggttgcgtttcgagacggccctga

SEQ ID NO: 516 - DNA, 236 bp, artificial
  oligo
  misc_feature (93)..(95): n is a, c, g, or t
  misc_feature (141)..(142): n is a, c, g, or t
gaccgcgtctcacaccgcggccatgcctgccagcttcgtttggagaccgtttaaacggtctctacctgcagcagtccgccgatttcggtttgnnngcaggtgcaatggcacatcacgcgaatacacctcattgcacctgcnntatgcatagatttcgcgcaccgtgtcgtttgaagagcgtttaaacgctcttcgcccctagcgcgccttccgagtatgtttcgagacggccctga

SEQ ID NO: 517 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctcgattccccgttacggtgatgtttgnnngcaggtgcaatgtcgcacgtacgatttactccccattgcacctgcnntatgcaggtacgtgtcatatacgtcggtttgaagagcgtttaaacgctcttcgcccgttgccggattacgtgtaatgtttcgagacggccctga

SEQ ID NO: 518 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctacctcgcatttcgcgctcgtatcggtttgnnngcaggtgcaatgccaggaagaacaacgtagacccattgcacctgcnntatgcacgttattccgagactacgtcgtttgaagagcgtttaaacgctcttcgcccttatacgcgccgaagtggatgtttcgagacggccctga

SEQ ID NO: 519 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctaccttgaatgaccagggtacctgcgtttgnnngcaggtgcaatgggcaccttctcacctatgaaccattgcacctgcnntatgcaattcttctctgtgaatgtcggtttgaagagcgtttaaacgctcttcgccccctatcatacgtatcctggtgtttcgagacggccctga

SEQ ID NO: 520 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctaccttgggactcctcggttatttagtttgnnngcaggtgcaatgagcctctaatccagctctacccattgcacctgcnntatgcactaaagggcgatctcaacaagtttgaagagcgtttaaacgctcttcgccctctgggcatgtgggtccggcgtttcgagacggccctga

SEQ ID NO: 521 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctacatagacccctgttgtaaggtttgnnngcaggtgcaatgcctcagcgacaaatcctaacccattgcacctgcnntatgcagctcaaaccattacagaagggtttgaagagcgtttaaacgctcttcgccccgtcaccaagccattgtaccgtttcgagacggccctga

SEQ ID NO: 522 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctcatcggcctgacccccatgcgtttgnnngcaggtgcaatgttacgcccaaaagcgacagagcattgcacctgcnntatgcacccaccaggatacgtatgatgtttgaagagcgtttaaacgctcttcgccctcatcctgcatgctagaccagtttcgagacggccctga

SEQ ID NO: 523 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctgctcaaaccattacagaagggtttgnnngcaggtgcaatgcccagactgaaagtactagcccattgcacctgcnntatgcagattccttccaataacacaagtttgaagagcgtttaaacgctcttcgcccctaaagggcgatctcaacaagtttcgagacggccctga

SEQ ID NO: 524 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctctgaaattagacatcaaagcgtttgnnngcaggtgcaatgcacatgcccgaattacctctccattgcacctgcnntatgcacaggcacgatagatacaaaggtttgaagagcgtttaaacgctcttcgccctgcccacaaagtatggcggtgtttcgagacggccctga

SEQ ID NO: 525 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctaccttgggactcctcggttatttagtttgnnngcaggtgcaatgccgaactgttacacaagcacgcattgcacctgcnntatgcagttacactctatctacacctgtttgaagagcgtttaaacgctcttcgccccctatcatacgtatcctggtgtttcgagacggccctga

SEQ ID NO: 526 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgattccttccaataacacaagtttgnnngcaggtgcaatgcgcgataccacgactagtacacattgcacctgcnntatgcatctgggcatgtgggtccggcgtttgaagagcgtttaaacgctcttcgcccagctgtagtagagtccccatgtttcgagacggccctga

SEQ ID NO: 527 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctaccttcacatggatggttactgaagtttgnnngcaggtgcaatgattcagtagaccgttcgtcggcattgcacctgcnntatgcaagcacacaagcacgttacccgtttgaagagcgtttaaacgctcttcgcccatcagttctgagcaactcgtgtttcgagacggccctga

SEQ ID NO: 528 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctaccttgtgatggtgaatattcacagtttgnnngcaggtgcaatggatcacgttgaaacccgactgcattgcacctgcnntatgcaccctcaccttctacccagccgtttgaagagcgtttaaacgctcttcgccctgcaagccttacctgtagcggtttcgagacggccctga

SEQ ID NO: 529 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctcagcttgtccaactggtcgggtttgnnngcaggtgcaatgtgtagtacccaggcagcagctcattgcacctgcnntatgcatgttgagatcgccctttagcgtttgaagagcgtttaaacgctcttcgccccaggcacgatagatacaaaggtttcgagacggccctga

SEQ ID NO: 530 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctttgggactcctcggttatttgtttgnnngcaggtgcaatggacttaacggaagttaccgcgcattgcacctgcnntatgcacgtcaccaagccattgtaccgtttgaagagcgtttaaacgctcttcgcccacacaacactgatgaggtccgtttcgagacggccctga

SEQ ID NO: 531 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctagcacacaagcacgttacccgtttgnnngcaggtgcaatgccagtctcaccaaacgaagggcattgcacctgcnntatgcaattcttctctgtgaatgtcggtttgaagagcgtttaaacgctcttcgccccagcttgtccaactggtcgggtttcgagacggccctga

SEQ ID NO: 532 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctagaagttggtgacgttctgcgtttgnnngcaggtgcaatgactaaggcccacagcgagaaacattgcacctgcnntatgcatgcccacaaagtatggcggtgtttgaagagcgtttaaacgctcttcgcccggccatttatagtgttgaccgtttcgagacggccctga

SEQ ID NO: 533 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctacaggtgacccaaccttcaggtttgnnngcaggtgcaatgtcggatactccaagctagctccattgcacctgcnntatgcactgcagttgagctggcaatcgtttgaagagcgtttaaacgctcttcgcccttgttacagccttattacacgtttcgagacggccctga

SEQ ID NO: 534 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctttgggactcctcggttatttgtttgnnngcaggtgcaatgtcagccgccaggaaaaagaaccattgcacctgcnntatgcacccaccaggatacgtatgatgtttgaagagcgtttaaacgctcttcgcccgtccgttgggtataacaacggtttcgagacggccctga

SEQ ID NO: 535 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctttgggactcctcggttatttgtttgnnngcaggtgcaatgccgtccaccagactatacgcacattgcacctgcnntatgcaggactgagagctgttgacacgtttgaagagcgtttaaacgctcttcgcccttgatcgggttcgtcggagcgtttcgagacggccctga

SEQ ID NO: 536 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctaccttcatcctgcatgctagaccagtttgnnngcaggtgcaatgcgactcatacatcgacagctgcattgcacctgcnntatgcagccctttagctggtatctgcgtttgaagagcgtttaaacgctcttcgcccagcgtaaacgtcgtccgcgagtttcgagacggccctga

SEQ ID NO: 537 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctaccttctttgaaccattgtgttatgtttgnnngcaggtgcaatggagcaatcacgatgaccacaccattgcacctgcnntatgcaagaagttggtgacgttctgcgtttgaagagcgtttaaacgctcttcgcccacgggccgttccggaccgttgtttcgagacggccctga

SEQ ID NO: 538 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctaccttgcaagccttacctgtagcggtttgnnngcaggtgcaatgtcacttccagccccaggaaatcattgcacctgcnntatgcaacacaacactgatgaggtccgtttgaagagcgtttaaacgctcttcgccctcgtaagcggtcgaaaacgagtttcgagacggccctga

SEQ ID NO: 539 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctatcagttctgagcaactcgtgtttgnnngcaggtgcaatgccgcaccttgttactaacccacattgcacctgcnntatgcactggaaggtgaggcctaggcgtttgaagagcgtttaaacgctcttcgccctagcgaccgtaccgccgattgtttcgagacggccctga

SEQ ID NO: 540 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctcaggcacgatagatacaaaggtttgnnngcaggtgcaatgctcacgagctaattgcctacccattgcacctgcnntatgcacgtcaccaagccattgtaccgtttgaagagcgtttaaacgctcttcgccccgcggtcgctagcgaaggaagtttcgagacggccctga

SEQ ID NO: 541 - DNA, 236 bp, artificial
  oligo
  misc_feature (93)..(95): n is a, c, g, or t
  misc_feature (141)..(142): n is a, c, g, or t
gaccgcgtctcacaccgtgttgagatcgccctttagcgtttggagaccgtttaaacggtctctacctgtgacttggtgtaagcacaagtttgnnngcaggtgcaatgcaaaccaccggatcgcactaccattgcacctgcnntatgcaacacaacactgatgaggtccgtttgaagagcgtttaaacgctcttcgccccgacgagtttgtctcgataggtttcgagacggccctga

SEQ ID NO: 542 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctacatagacccctgttgtaaggtttgnnngcaggtgcaatgccatacgttagaccctaaccgcattgcacctgcnntatgcagtgacttggtgtaagcacaagtttgaagagcgtttaaacgctcttcgccccggatgttacctcggtagacgtttcgagacggccctga

SEQ ID NO: 543 - DNA, 236 bp, artificial
  oligo
  misc_feature (93)..(95): n is a, c, g, or t
  misc_feature (141)..(142): n is a, c, g, or t
gaccgcgtctcacaccggttacactctatctacacctgtttggagaccgtttaaacggtctctacctcaacttcttcgtggtatctcgtttgnnngcaggtgcaatgtactatgacgaccaccgcctccattgcacctgcnntatgcatcacatggatggttactgaagtttgaagagcgtttaaacgctcttcgccctcgtatcgggcgaattcctagtttcgagacggccctga

SEQ ID NO: 544 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctacatagacccctgttgtaaggtttgnnngcaggtgcaatggatcagcacgaaaactagcggcattgcacctgcnntatgcatgaatgaccagggtacctgcgtttgaagagcgtttaaacgctcttcgccccccgtatcgccgagcaaactgtttcgagacggccctga

SEQ ID NO: 545 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctcagcttgtccaactggtcgggtttgnnngcaggtgcaatgctgacgctacagacactagaccattgcacctgcnntatgcattgttacagccttattacacgtttgaagagcgtttaaacgctcttcgccccgggattatcgatggacggtgtttcgagacggccctga

SEQ ID NO: 546 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttgttgggcagctgtatgatcgtttgnnngcaggtgcaatggccccatctctgaaatccactcattgcacctgcnntatgcatgcaagccttacctgtagcggtttgaagagcgtttaaacgctcttcgcccgacgatcgcgcgcatatatcgtttcgagacggccctga

SEQ ID NO: 547 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctattcttctctgtgaatgtcggtttgnnngcaggtgcaatgcgcattgtcaacgcaacttcccattgcacctgcnntatgcacaatacagggatacccactagtttgaagagcgtttaaacgctcttcgcccaccgattccccgttcggactgtttcgagacggccctga

SEQ ID NO: 548 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgtcgatggcaatagccaaggtttgnnngcaggtgcaatgggatttaacacgctacgagcgcattgcacctgcnntatgcaggactgagagctgttgacacgtttgaagagcgtttaaacgctcttcgcccgagcgtcgtcgcacactagtgtttcgagacggccctga

SEQ ID NO: 549 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctctgaaattagacatcaaagcgtttgnnngcaggtgcaatgcgctctcctacagactatagccattgcacctgcnntatgcatgttgggcagctgtatgatcgtttgaagagcgtttaaacgctcttcgcccttgtacgcttcgggcgcgatgtttcgagacggccctga

SEQ ID NO: 550 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctaaagggcgatctcaacaagtttgnnngcaggtgcaatgactttcctatgtccgccactgcattgcacctgcnntatgcatgcccacaaagtatggcggtgtttgaagagcgtttaaacgctcttcgccccctgctacgcgtctacacgtgtttcgagacggccctga

SEQ ID NO: 551 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctacaggtgacccaaccttcaggtttgnnngcaggtgcaatgtacgcacatcccatcctagagcattgcacctgcnntatgcaacctatcatacgtatcctgggtttgaagagcgtttaaacgctcttcgcccaccgactgcgatctaaccgcgtttcgagacggccctga

SEQ ID NO: 552 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttgtcgatggcaatagccaaggtttgnnngcaggtgcaatgcgtgagacaaactcaaccggacattgcacctgcnntatgcattgttacagccttattacacgtttgaagagcgtttaaacgctcttcgccctcaacgcgcgtagggcggatgtttcgagacggccctga

SEQ ID NO: 553 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcgtcaccaagccattgtaccgtttgnnngcaggtgcaatggagctactcgcctttagatgccattgcacctgcnntatgcaactttccatgtcagccatacgtttgaagagcgtttaaacgctcttcgcccagtacgtcccgatggtatcggtttcgagacggccctga

SEQ ID NO: 554 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctaccttgaatgaccagggtacctgcgtttgnnngcaggtgcaatgtggcttgtcgtgacgaaacaccattgcacctgcnntatgcaggccgcggataatttcgccagtttgaagagcgtttaaacgctcttcgccctccccgtcggagtcgtacgcgtttcgagacggccctga

SEQ ID NO: 555 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctggccatttatagtgttgaccgtttgnnngcaggtgcaatggagatccaccgcattaagtgccattgcacctgcnntatgcagacttcgacaccggccgtttgtttgaagagcgtttaaacgctcttcgccctatgctacgctcgtcgtgcggtttcgagacggccctga

SEQ ID NO: 556 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctgattccttccaataacacaagtttgnnngcaggtgcaatgctccactagagccacttccgacattgcacctgcnntatgcaactatgcggtaggtacgccggtttgaagagcgtttaaacgctcttcgcccgtgcggacgttacacccgatgtttcgagacggccctga

SEQ ID NO: 557 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctggaagggacactaacctggtgtttgnnngcaggtgcaatggacgcagcacataggtaagtgcattgcacctgcnntatgcaagcggcgtaagacgattatcgtttgaagagcgtttaaacgctcttcgccccctcatgaccgctcgccgatgtttcgagacggccctga

SEQ ID NO: 558 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcatcggcctgacccccatgcgtttgnnngcaggtgcaatggaccaacacaatctcgtgacgcattgcacctgcnntatgcatacgaatcgtcagagcggatgtttgaagagcgtttaaacgctcttcgcccgctacggtagacgtcgacaagtttcgagacggccctga

SEQ ID NO: 559 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcagcttgtccaactggtcgggtttgnnngcaggtgcaatgcactagaattgcagcaacgcccattgcacctgcnntatgcagtcccttactacgtacgcgggtttgaagagcgtttaaacgctcttcgcccaaggctcgatagttcgcgacgtttcgagacggccctga

SEQ ID NO: 560 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctcaatacagggatacccactagtttgnnngcaggtgcaatgaccttccagcctaacctcaagcattgcacctgcnntatgcacgaacgatatccgaatgcacgtttgaagagcgtttaaacgctcttcgccctcggtactttgaaaccgcgcgtttcgagacggccctga

SEQ ID NO: 561 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctctgcagttgagctggcaatcgtttgnnngcaggtgcaatgtaatatcgtcccctacctcggcattgcacctgcnntatgcatgtaacgtcttaagccgcgggtttgaagagcgtttaaacgctcttcgcccggtttgcgctaacgtgtggcgtttcgagacggccctga

SEQ ID NO: 562 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctccctcaccttctacccagccgtttgnnngcaggtgcaatggctgggaaagctattacctcgcattgcacctgcnntatgcacgaacgacgaggtactagcggtttgaagagcgtttaaacgctcttcgcccaactcgcttcgtcggatatagtttcgagacggccctga

SEQ ID NO: 563 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctctgctagccaacaccactgagtttgnnngcaggtgcaatgacaggaaatcagccaagaccgcattgcacctgcnntatgcagttcgtcaagtccgccgcaggtttgaagagcgtttaaacgctcttcgcccccgcccacgatattggcggagtttcgagacggccctga

SEQ ID NO: 564 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttcacccataggagccacagcgtttgnnngcaggtgcaatgtagcagggtaccgtactgacacattgcacctgcnntatgcaaacgatagcgcgcttaagcggtttgaagagcgtttaaacgctcttcgcccggttgaccgcgaccgttcgggtttcgagacggccctga

SEQ ID NO: 565 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctagcacacaagcacgttacccgtttgnnngcaggtgcaatgtctttcctctaagcccccagacattgcacctgcnntatgcactcgacgcaaccgcagatcggtttgaagagcgtttaaacgctcttcgccccgtttaagttggtaccccgcgtttcgagacggccctga

SEQ ID NO: 566 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctacatagacccctgttgtaaggtttgnnngcaggtgcaatgtaggaactcacccgtaagccgcattgcacctgcnntatgcaatattgccggcacgatagatgtttgaagagcgtttaaacgctcttcgccctgacaacgcggatttggtcggtttcgagacggccctga

SEQ ID NO: 567 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctacacaacactgatgaggtccgtttgnnngcaggtgcaatgtgactacacctacagcgcacccattgcacctgcnntatgcaccgccagttgcaatcgtacagtttgaagagcgtttaaacgctcttcgccccgccatacgaggcgaaagcagtttcgagacggccctga

SEQ ID NO: 568 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctagctgtagtagagtccccatgtttgnnngcaggtgcaatgagcccgaaataacctgaagggcattgcacctgcnntatgcattgtgaccgttcgatgttcggtttgaagagcgtttaaacgctcttcgccctgacgcccctcgacggcatagtttcgagacggccctga

SEQ ID NO: 569 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctgatccactcgcggaacttacgtttgnnngcaggtgcaatgcaccgcagcttacatacaggacattgcacctgcnntatgcatacgatccgcgcgcgcgggagtttgaagagcgtttaaacgctcttcgcccagtcgttcggcccgtctagtgtttcgagacggccctga

SEQ ID NO: 570 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctcggacgtcccgtatcgggatgtttgnnngcaggtgcaatgcaggcaactcagtcacctatccattgcacctgcnntatgcaatcccgtggcgatgtatcgagtttgaagagcgtttaaacgctcttcgccctcgcgaacgctcggagatgtgtttcgagacggccctga

SEQ ID NO: 571 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctcgttgtagtccgcgcgcgacgtttgnnngcaggtgcaatggaaacgcacctcaagaaacggcattgcacctgcnntatgcaccgaatgtgcgctaaatcgagtttgaagagcgtttaaacgctcttcgcccgttaaactaaatcggcgcgcgtttcgagacggccctga

SEQ ID NO: 572 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctaccttgcgtcaatcgcagcgtctagtttgnnngcaggtgcaatgccgttccaaacacaaacacggcattgcacctgcnntatgcacttaaattcccgcgccgcgagtttgaagagcgtttaaacgctcttcgccccaggtacggccgacgagctagtttcgagacggccctga

SEQ ID NO: 573 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctgcgcgacacatgggcggttcgtttgnnngcaggtgcaatgcccatagcccctaagattctccattgcacctgcnntatgcatagacggacgttactgcacggtttgaagagcgtttaaacgctcttcgccctcgacgacgataaaagcgctgtttcgagacggccctga

SEQ ID NO: 574 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctaccttccgcatttcgcgcgtgcaagtttgnnngcaggtgcaatggcatactctgctactcgaccacattgcacctgcnntatgcaccgaataccgagtcgttacggtttgaagagcgtttaaacgctcttcgcccatgcgcgcgctggcgctgtagtttcgagacggccctga

SEQ ID NO: 575 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctccacagtactcacttggtccgtttgnnngcaggtgcaatgccgaccggaaaacccttactgcattgcacctgcnntatgcagagaagcactatgggggcatgtttgaagagcgtttaaacgctcttcgccctggcattggggacctcgggtgtttcgagacggccctga

SEQ ID NO: 576 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttcacacttggagttacagcagtttgnnngcaggtgcaatgagatgttgcacacccaactcccattgcacctgcnntatgcaaccaggctgacagttatcacgtttgaagagcgtttaaacgctcttcgcccgtgtctgtatgacccacccggtttcgagacggccctga

SEQ ID NO: 577 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctatccccgtcgcagttgaactgtttgnnngcaggtgcaatgcctgattaagcacttagccgccattgcacctgcnntatgcattattcttgtccaggcaagggtttgaagagcgtttaaacgctcttcgccctaagctgctctctagacggagtttcgagacggccctga

SEQ ID NO: 578 - DNA, 236 bp, artificial
  oligo
  misc_feature (93)..(95): n is a, c, g, or t
  misc_feature (141)..(142): n is a, c, g, or t
gaccgcgtctcacaccgccacagtactcacttggtccgtttggagaccgtttaaacggtctctaccttaaccatagtacccagttgtgtttgnnngcaggtgcaatgccccagctcagaaagacttagcattgcacctgcnntatgcagtcacacctgagtaccactggtttgaagagcgtttaaacgctcttcgcccgtggaagtctgtgtcgggaagtttcgagacggccctga

SEQ ID NO: 579 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttcacacttggagttacagcagtttgnnngcaggtgcaatgctcgaataatagacgctgccgcattgcacctgcnntatgcataagctgctctctagacggagtttgaagagcgtttaaacgctcttcgcccggggcatgggccatgctgtagtttcgagacggccctga

SEQ ID NO: 580 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctgtgcatacgtacttcgtccagtttgnnngcaggtgcaatgctgacacccaacgaagtgactcattgcacctgcnntatgcagttcactacgagtgtattccgtttgaagagcgtttaaacgctcttcgcccgtatatgccgctacgcctttgtttcgagacggccctga

SEQ ID NO: 581 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctgcaaagccgatgtcagcgccgtttgnnngcaggtgcaatgctcctgttccatctataccggcattgcacctgcnntatgcatcctcagctgccatatgtgcgtttgaagagcgtttaaacgctcttcgcccgcgaccgcacttccgttctagtttcgagacggccctga

SEQ ID NO: 582 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctagccacgcttgctgatgttggtttgnnngcaggtgcaatgacggacgaatgtaaaagcccccattgcacctgcnntatgcaatccccgtcgcagttgaactgtttgaagagcgtttaaacgctcttcgccctaccgtacgtggaactccgcgtttcgagacggccctga

SEQ ID NO: 583 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttaccttgagactccaccctcgtttgnnngcaggtgcaatgacaacaccatattgagcggcgcattgcacctgcnntatgcagtagaccaggcacccaacctgtttgaagagcgtttaaacgctcttcgcccattcgcgttatcgaacacgtgtttcgagacggccctga

SEQ ID NO: 584 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttgctgtcgtgagtgccagccgtttgnnngcaggtgcaatgcacatgtccttacgccaatcccattgcacctgcnntatgcagagaggtttccgaagactaagtttgaagagcgtttaaacgctcttcgccccggacgtagcaaccgttgaagtttcgagacggccctga

SEQ ID NO: 585 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctctagcaagccataacaaaacgtttgnnngcaggtgcaatggcgcgacaactcaagttatgccattgcacctgcnntatgcaaaccccaacatcagcaagcggtttgaagagcgtttaaacgctcttcgccccgattccccgttacggtgatgtttcgagacggccctga

SEQ ID NO: 586 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctaagcactacccctgccaaccgtttgnnngcaggtgcaatgcctaagaccgcacccattgaccattgcacctgcnntatgcacagcttcaggaccccgatccgtttgaagagcgtttaaacgctcttcgcccttcaactaacgcgcccgctagtttcgagacggccctga

SEQ ID NO: 587 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctttcttctcagttcaagtaacgtttgnnngcaggtgcaatgagccaggaccacaattcccatcattgcacctgcnntatgcatcccacagttgtaccttatcgtttgaagagcgtttaaacgctcttcgccctttggccgcgcgctcgatacgtttcgagacggccctga

SEQ ID NO: 588 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctgccatgggcaccttctccgcgtttgnnngcaggtgcaatgcgaacaacttcctccacttcccattgcacctgcnntatgcatgtgaaaaggactgcaggccgtttgaagagcgtttaaacgctcttcgcccttatcgcgtcgtgatcggcagtttcgagacggccctga

SEQ ID NO: 589 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttcctgccgaggttgggtgccgtttgnnngcaggtgcaatgcgataataccacccacggtcgcattgcacctgcnntatgcagtcacacctgagtaccactggtttgaagagcgtttaaacgctcttcgccccgaagttgccgaatgcgaatgtttcgagacggccctga

SEQ ID NO: 590 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctatcttgcagatgctaatagcgtttgnnngcaggtgcaatgcgaacccgctacagcttagtccattgcacctgcnntatgcaccgtcgttcccttacggatagtttgaagagcgtttaaacgctcttcgcccgggaccgatccattaccgtagtttcgagacggccctga

SEQ ID NO: 591 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctaccttaaccatagtacccagttgtgtttgnnngcaggtgcaatggcgcaacacggtgaaaagatgcattgcacctgcnntatgcacgccgaatattagcccgaacgtttgaagagcgtttaaacgctcttcgccctgcgattgcggcgactaaacgtttcgagacggccctga

SEQ ID NO: 592 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgagaagcactatgggggcatgtttgnnngcaggtgcaatgcgaacccttgtgccaacataccattgcacctgcnntatgcattaagcgaccgcgatactccgtttgaagagcgtttaaacgctcttcgcccaccgaccaaattccccggcggtttcgagacggccctga

SEQ ID NO: 593 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttggcattggggacctcgggtgtttgnnngcaggtgcaatgcctatatggtccctatccccacattgcacctgcnntatgcacggctatacggtgtagcaacgtttgaagagcgtttaaacgctcttcgcccgataccgaaacgatgtcgtcgtttcgagacggccctga

SEQ ID NO: 594 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctgagccgctgtgatcataccagtttgnnngcaggtgcaatgcccctagcaatatggacaacgcattgcacctgcnntatgcaatcgcgttgttgcatgacgggtttgaagagcgtttaaacgctcttcgcccacgcgcgtaacctgtcgcacgtttcgagacggccctga

SEQ ID NO: 595 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgccatgggcaccttctccgcgtttgnnngcaggtgcaatgacacctgttcacaactccaggcattgcacctgcnntatgcagggccgtgcacgcgttttaggtttgaagagcgtttaaacgctcttcgccctagcccgatccgagcgcatagtttcgagacggccctga

SEQ ID NO: 596 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctacctgtggaagtctgtgtcgggaagtttgnnngcaggtgcaatgcgaatccgcgacattctaccgcattgcacctgcnntatgcagatcgacaaatataacgcgcgtttgaagagcgtttaaacgctcttcgcccggacgcccggcgcgtctaacgtttcgagacggccctga

SEQ ID NO: 597 - DNA, 236 bp, artificial
  oligo
  misc_feature (93)..(95): n is a, c, g, or t
  misc_feature (141)..(142): n is a, c, g, or t
gaccgcgtctcacaccgtaccttgagactccaccctcgtttggagaccgtttaaacggtctctaccttcacacttggagttacagcagtttgnnngcaggtgcaatgctcgacaccttaaagcggcaacattgcacctgcnntatgcactagcggcgcaatacgtgctgtttgaagagcgtttaaacgctcttcgcccgtcggcgataagatggacgcgtttcgagacggccctga

SEQ ID NO: 598 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctttcctacaaccttctcgcaagtttgnnngcaggtgcaatggccccgaactcaatcattccacattgcacctgcnntatgcacgaccggccacatcgactaggtttgaagagcgtttaaacgctcttcgccccgttacgcctccgccgttccgtttcgagacggccctga

SEQ ID NO: 599 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctaccttacgctgcaaagtctctggcgtttgnnngcaggtgcaatgcccccaactaaagtagtccgacattgcacctgcnntatgcaacgacggcattcatctcgatgtttgaagagcgtttaaacgctcttcgccccgctcgtggctacgacgattgtttcgagacggccctga

SEQ ID NO: 600 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctacctctccggcgaccggaaatatagtttgnnngcaggtgcaatggcacaccaagcacgatatcagcattgcacctgcnntatgcatgcgcgatgatctaacgcacgtttgaagagcgtttaaacgctcttcgccctagatttcgcgcaccgtgtcgtttcgagacggccctga

SEQ ID NO: 601 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctccgaagtgtatcccgatccggtttgnnngcaggtgcaatgcgagaaaacccgtatcatcgccattgcacctgcnntatgcataagcgacgccgatcataccgtttgaagagcgtttaaacgctcttcgccctatggcgcgctcgattcagggtttcgagacggccctga

SEQ ID NO: 602 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctacggttcattcgataaggatgtttgnnngcaggtgcaatgaacacgcacctttatgaccggcattgcacctgcnntatgcatctacggccgcggttacgttgtttgaagagcgtttaaacgctcttcgccctctctatccgttcgcgcgcggtttcgagacggccctga

SEQ ID NO: 603 - DNA, 236 bp, artificial
  oligo
  misc_feature (93)..(95): n is a, c, g, or t
  misc_feature (141)..(142): n is a, c, g, or t
gaccgcgtctcacaccgtaccttgagactccaccctcgtttggagaccgtttaaacggtctctacctgatagctcaagcgcgacgaagtttgnnngcaggtgcaatggagcacatcgaatccgtttcccattgcacctgcnntatgcacggaccttgtagccgcgtacgtttgaagagcgtttaaacgctcttcgccccgagcgtcgaaactcgacgcgtttcgagacggccctga

SEQ ID NO: 604 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctcgcgcgccgagggctgtattgtttgnnngcaggtgcaatggcttgtccaaatacagtcccccattgcacctgcnntatgcaagttgcttcgttcgaccagtgtttgaagagcgtttaaacgctcttcgcccgaccgcgtagcaattcgtatgtttcgagacggccctga

SEQ ID NO: 605 - DNA, 236 bp, artificial
  oligo
  misc_feature (93)..(95): n is a, c, g, or t
  misc_feature (141)..(142): n is a, c, g, or t
gaccgcgtctcacaccgcgcccagggagcgcaaacacgtttggagaccgtttaaacggtctctacctatcctccacgtgcggttcccgtttgnnngcaggtgcaatggctcgatacgatttgaacccgcattgcacctgcnntatgcaccgagttacctcgagctttcgtttgaagagcgtttaaacgctcttcgcccctcgtagcttgccacactgtgtttcgagacggccctga

SEQ ID NO: 606 - DNA, 236 bp, artificial
  oligo
  misc_feature (93)..(95): n is a, c, g, or t
  misc_feature (141)..(142): n is a, c, g, or t
gaccgcgtctcacaccgtcgggggacggttatttctggtttggagaccgtttaaacggtctctaccttcagaagcatgataagcaccgtttgnnngcaggtgcaatgacagactagcactaacaccgccattgcacctgcnntatgcacggccatgcctgccagcttcgtttgaagagcgtttaaacgctcttcgcccgacttcactagagcgccgtagtttcgagacggccctga

SEQ ID NO: 607 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctctcctgggaaccgcacgtgggtttgnnngcaggtgcaatgaccgtccctattgaaacgccccattgcacctgcnntatgcacgcgcaccatagcatcacgcgtttgaagagcgtttaaacgctcttcgcccttatcgacaatccgctcactgtttcgagacggccctga

SEQ ID NO: 608 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctttgggggatactcacgggatgtttgnnngcaggtgcaatgcgtgcacaccaagttccgatacattgcacctgcnntatgcaccgagttacctcgagctttcgtttgaagagcgtttaaacgctcttcgcccatccgtcgcgctaacgacgggtttcgagacggccctga

SEQ ID NO: 609 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctaccttgataagcacctggtgctccgtttgnnngcaggtgcaatgccacgtacgaataaactgccgcattgcacctgcnntatgcatcgacaacccctacatcgtggtttgaagagcgtttaaacgctcttcgccccgttcggctaggccaatctggtttcgagacggccctga

SEQ ID NO: 610 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctaccttcgacaacccctacatcgtggtttgnnngcaggtgcaatgcacgacgcacatctttggcttcattgcacctgcnntatgcaaacactacgtccctcgtactgtttgaagagcgtttaaacgctcttcgcccaatatccgttagcggggtcggtttcgagacggccctga

SEQ ID NO: 611 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctttctcggaagtcgaggacaggtttgnnngcaggtgcaatgacccgccacatctaggtaatgcattgcacctgcnntatgcacggaccaatcatcatcggatgtttgaagagcgtttaaacgctcttcgccctgagtcgcgatatatgccgagtttcgagacggccctga

SEQ ID NO: 612 - DNA, 236 bp, artificial
  oligo
  misc_feature (93)..(95): n is a, c, g, or t
  misc_feature (141)..(142): n is a, c, g, or t
gaccgcgtctcacaccgcattgtatacgccttcctgggtttggagaccgtttaaacggtctctacctactcacggcagcgcacgcacgtttgnnngcaggtgcaatgccccatgcgacacgtatttcgcattgcacctgcnntatgcagccgccgtagtaaaaccgtcgtttgaagagcgtttaaacgctcttcgcccgctcgcgctatcatgcgttggtttcgagacggccctga

SEQ ID NO: 613 - DNA, 236 bp, artificial
  oligo
  misc_feature (93)..(95): n is a, c, g, or t
  misc_feature (141)..(142): n is a, c, g, or t
gaccgcgtctcacaccgggccagaatacaggtcccgcgtttggagaccgtttaaacggtctctacctcgcgcaccatagcatcacgcgtttgnnngcaggtgcaatgaatcagagccatccgactacgcattgcacctgcnntatgcagctatcgcttgccggagccggtttgaagagcgtttaaacgctcttcgcccatctgtattaacgcggtacagtttcgagacggccctga

SEQ ID NO: 614 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctggggcacgatggcctttaccgtttgnnngcaggtgcaatgatcagacacgacgatctccaccattgcacctgcnntatgcagggcgcgcatgacgaccgaagtttgaagagcgtttaaacgctcttcgcccgaatctcgcgtcacgtaatcgtttcgagacggccctga

SEQ ID NO: 615 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctgtgacccgaggaaccggtgcgtttgnnngcaggtgcaatgagaacattcctctccgactggcattgcacctgcnntatgcacgtatcgtatctgacgtgtggtttgaagagcgtttaaacgctcttcgccccggtcgactggtcgatcattgtttcgagacggccctga

SEQ ID NO: 616 - DNA, 236 bp, artificial
  oligo
  misc_feature (93)..(95): n is a, c, g, or t
  misc_feature (141)..(142): n is a, c, g, or t
gaccgcgtctcacaccggccttgagcccggtggggctgtttggagaccgtttaaacggtctctacctctcgtagttcaaacgacgtcgtttgnnngcaggtgcaatggcacgcagaaacccattgatccattgcacctgcnntatgcagattttcgcgcattacgcaggtttgaagagcgtttaaacgctcttcgccctaaaacgccgcgtcggaatggtttcgagacggccctga

SEQ ID NO: 617 - DNA, 236 bp, artificial
  oligo
  misc_feature (93)..(95): n is a, c, g, or t
  misc_feature (141)..(142): n is a, c, g, or t
gaccgcgtctcacaccgtcgacaacccctacatcgtggtttggagaccgtttaaacggtctctaccttcagcgggccgacatcgacggtttgnnngcaggtgcaatgcgcaagtacccagatgatccacattgcacctgcnntatgcagatcgctaggattgcgcggggtttgaagagcgtttaaacgctcttcgcccaaacggtcgccggccggagtgtttcgagacggccctga

SEQ ID NO: 618 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctgtgcgctaatagatgcgcgggtttgnnngcaggtgcaatgagcacccgcacttgactttaccattgcacctgcnntatgcaaatcgtatttggtcgcgcgagtttgaagagcgtttaaacgctcttcgcccgctctcgataccgccgtgtcgtttcgagacggccctga

SEQ ID NO: 619 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctcgtgacgaggctataccggtgtttgnnngcaggtgcaatgccccaagaacgaggctaaatccattgcacctgcnntatgcacgcgtcgctagtgtgtccatgtttgaagagcgtttaaacgctcttcgcccccacgattcgcgcgaatcctgtttcgagacggccctga

SEQ ID NO: 620 - DNA, 236 bp, artificial
  oligo
  misc_feature (93)..(95): n is a, c, g, or t
  misc_feature (141)..(142): n is a, c, g, or t
gaccgcgtctcacaccgtcgatcggatgacggtatgtgtttggagaccgtttaaacggtctctacctctgtccgttcgcgtagattagtttgnnngcaggtgcaatggaccagttcaactgcactcgacattgcacctgcnntatgcagacccgaactgcgcgcgatagtttgaagagcgtttaaacgctcttcgcccaacactacgtccctcgtactgtttcgagacggccctga

SEQ ID NO: 621 - DNA, 236 bp, artificial
  oligo
  misc_feature (93)..(95): n is a, c, g, or t
  misc_feature (141)..(142): n is a, c, g, or t
gaccgcgtctcacaccgaccgccgaatcgagcctgcggtttggagaccgtttaaacggtctctaccttcgtggcgggtcgaattcgcgtttgnnngcaggtgcaatgtgacccgatttaacccgccatcattgcacctgcnntatgcacaacgaccggtacggatcatgtttgaagagcgtttaaacgctcttcgcccagataatatgcgccgctcgggtttcgagacggccctga

SEQ ID NO: 622 - DNA, 236 bp, artificial
  oligo
  misc_feature (93)..(95): n is a, c, g, or t
  misc_feature (141)..(142): n is a, c, g, or t
gaccgcgtctcacaccggggcgtatcggtcaagacgagtttggagaccgtttaaacggtctctacctatcactaatgaatcgcggtagtttgnnngcaggtgcaatggcgaccaccgtaattgccaaacattgcacctgcnntatgcaaggacgcgcgtaacctccacgtttgaagagcgtttaaacgctcttcgcccaacaagcggctatgtcggacgtttcgagacggccctga

SEQ ID NO: 623 - DNA, 236 bp, artificial
  oligo
  misc_feature (93)..(95): n is a, c, g, or t
  misc_feature (141)..(142): n is a, c, g, or t
gaccgcgtctcacaccgcaagcgcgttagcggtcgtcgtttggagaccgtttaaacggtctctacctaaaccgatcgcgagtgcgcggtttgnnngcaggtgcaatgtagcgcaacctacgtttcacccattgcacctgcnntatgcagtcggcgataacgcatagctgtttgaagagcgtttaaacgctcttcgcccccgcggcggataacgtgtttgtttcgagacggccctga

SEQ ID NO: 624 - DNA, 236 bp, artificial
  oligo
  misc_feature (93)..(95): n is a, c, g, or t
  misc_feature (141)..(142): n is a, c, g, or t
gaccgcgtctcacaccgataacgccgcgaccaacgtagtttggagaccgtttaaacggtctctacctatagccttgcgtcgagtatggtttgnnngcaggtgcaatggctaacctattggacgactcccattgcacctgcnntatgcacgaagcgggatttacgtctagtttgaagagcgtttaaacgctcttcgcccgcttacggacccccccgatagtttcgagacggccctga

SEQ ID NO: 625 - DNA, 236 bp, artificial
  oligo
  misc_feature (93)..(95): n is a, c, g, or t
  misc_feature (141)..(142): n is a, c, g, or t
gaccgcgtctcacaccggtcggcgataagatggacgcgtttggagaccgtttaaacggtctctacctgcgcgaagcgtatttggcgggtttgnnngcaggtgcaatggtatccctatagcccaatcggcattgcacctgcnntatgcatagacctcgacgaacgctttgtttgaagagcgtttaaacgctcttcgcccctgcgctcgcttaaggttaggtttcgagacggccctga

SEQ ID NO: 626 - DNA, 236 bp, artificial
  oligo
  misc_feature (93)..(95): n is a, c, g, or t
  misc_feature (141)..(142): n is a, c, g, or t
gaccgcgtctcacaccgggttctacgcccgcggctcagtttggagaccgtttaaacggtctctacctgggaccgatccattaccgtagtttgnnngcaggtgcaatgcaaaggagcacacgcctcaatcattgcacctgcnntatgcagataccgacctctcgccgcggtttgaagagcgtttaaacgctcttcgccccaagtagcgcgcttagacatgtttcgagacggccctga

SEQ ID NO: 627 - DNA, 236 bp, artificial
  oligo
  misc_feature (93)..(95): n is a, c, g, or t
  misc_feature (141)..(142): n is a, c, g, or t
gaccgcgtctcacaccgtcgaacgcgaagggtcgcgtgtttggagaccgtttaaacggtctctacctactaacgacgtcgactagccgtttgnnngcaggtgcaatgggtagacaccagacctaacagcattgcacctgcnntatgcagcgagtcgagcaatttggcggtttgaagagcgtttaaacgctcttcgcccctattacctttcgacgcgatgtttcgagacggccctga

SEQ ID NO: 628 - DNA, 236 bp, artificial
  oligo
  misc_feature (93)..(95): n is a, c, g, or t
  misc_feature (141)..(142): n is a, c, g, or t
gaccgcgtctcacaccgacgaaccggtctaccgcatagtttggagaccgtttaaacggtctctacctcgagcgtcgaaactcgacgcgtttgnnngcaggtgcaatgccccagataagtcctcactcacattgcacctgcnntatgcatcgcctaccgacgcgagattgtttgaagagcgtttaaacgctcttcgcccggtcgctcgatgcacgacatgtttcgagacggccctga

SEQ ID NO: 629 - DNA, 236 bp, artificial
  oligo
  misc_feature (93)..(95): n is a, c, g, or t
  misc_feature (141)..(142): n is a, c, g, or t
gaccgcgtctcacaccggagcgttacggcacgacgccgtttggagaccgtttaaacggtctctacctacccgtattcggacgtaccggtttgnnngcaggtgcaatgccgcactcatctcgttctctacattgcacctgcnntatgcaccggtcgaggcgactactccgtttgaagagcgtttaaacgctcttcgcccaataacggcgcgttaagcgtgtttcgagacggccctga

SEQ ID NO: 630 - DNA, 236 bp, artificial
  oligo
  misc_feature (93)..(95): n is a, c, g, or t
  misc_feature (141)..(142): n is a, c, g, or t
gaccgcgtctcacaccgaaccggcggtacgcgcgtttgtttggagaccgtttaaacggtctctacctatagacgcgtgacaaccgaagtttgnnngcaggtgcaatggaccatcagacaacccgtcaacattgcacctgcnntatgcacgtcttatagtaatcggcgggtttgaagagcgtttaaacgctcttcgcccttcgcggcggagaagttagggtttcgagacggccctga

SEQ ID NO: 631 - DNA, 236 bp, artificial
  oligo
  misc_feature (93)..(95): n is a, c, g, or t
  misc_feature (141)..(142): n is a, c, g, or t
gaccgcgtctcacaccggacgccttaatcatcgagatgtttggagaccgtttaaacggtctctacctacgcaatcagcccaagcgttgtttgnnngcaggtgcaatgcagtaatccatcgacgctccccattgcacctgcnntatgcacgcattgaccgacccgtcgagtttgaagagcgtttaaacgctcttcgccctagtcgcaccgtaagccgaggtttcgagacggccctga

SEQ ID NO: 632 - DNA, 236 bp, artificial
  oligo
  misc_feature (93)..(95): n is a, c, g, or t
  misc_feature (141)..(142): n is a, c, g, or t
gaccgcgtctcacaccgagtacgtcccgatggtatcggtttggagaccgtttaaacggtctctacctcgaaactatggccggcgaatgtttgnnngcaggtgcaatggtcctaacaccttcgtacgtccattgcacctgcnntatgcacgcgaagcgttgaacgacaggtttgaagagcgtttaaacgctcttcgcccgacatcccgccgccgttgttgtttcgagacggccctga

SEQ ID NO: 633 - DNA, 236 bp, artificial
  oligo
  misc_feature (93)..(95): n is a, c, g, or t
  misc_feature (141)..(142): n is a, c, g, or t
gaccgcgtctcacaccgacggttcattcgataaggatgtttggagaccgtttaaacggtctctacctcccggcggttcgtcttcgccgtttgnnngcaggtgcaatggtgcagcagtgtgttctaggacattgcacctgcnntatgcagcgatttgcgtgacgcacgggtttgaagagcgtttaaacgctcttcgcccgctaaaccggctcgggctatgtttcgagacggccctga

SEQ ID NO: 634 - DNA, 236 bp, artificial
  oligo
  misc_feature (93)..(95): n is a, c, g, or t
  misc_feature (141)..(142): n is a, c, g, or t
gaccgcgtctcacaccgagcgcgctagcgcagagtacgtttggagaccgtttaaacggtctctacctccccgactcgagtaacgatcgtttgnnngcaggtgcaatggcctgcacctacctccagtaacattgcacctgcnntatgcaccgtacgactatctaaagctgtttgaagagcgtttaaacgctcttcgccctaggcggcttatgtccggttgtttcgagacggccctga

SEQ ID NO: 635 - DNA, 236 bp, artificial
  oligo
  misc_feature (93)..(95): n is a, c, g, or t
  misc_feature (141)..(142): n is a, c, g, or t
gaccgcgtctcacaccgatacgagactcttgcgcgacgtttggagaccgtttaaacggtctctacctagtaatgcacccgtcgatccgtttgnnngcaggtgcaatgctctacgacgaagagaacccccattgcacctgcnntatgcaatacgcgaatgcgttgacacgtttgaagagcgtttaaacgctcttcgccccgattcgcgtactgagccgagtttcgagacggccctga

SEQ ID NO: 636 - DNA, 236 bp, artificial
  oligo
  misc_feature (93)..(95): n is a, c, g, or t
  misc_feature (141)..(142): n is a, c, g, or t
gaccgcgtctcacaccgtattcgttcgccgcgcgactgtttggagaccgtttaaacggtctctacctacgcgcccggttacctcggagtttgnnngcaggtgcaatgcgcgcctataaattacacggccattgcacctgcnntatgcaaaacggcggatctaggaacggtttgaagagcgtttaaacgctcttcgccctatcgcgaagtgccgtgagtgtttcgagacggccctga

SEQ ID NO: 637 - DNA, 236 bp, artificial
  oligo
  misc_feature (93)..(95): n is a, c, g, or t
  misc_feature (141)..(142): n is a, c, g, or t
gaccgcgtctcacaccgtcgagacaaattcgtcgcgtgtttggagaccgtttaaacggtctctacctctctaaacgtcgaaccgtccgtttgnnngcaggtgcaatgagtgtggacgaaccccttcaccattgcacctgcnntatgcaactgcgcgtataacgatagggtttgaagagcgtttaaacgctcttcgccccgcacgccgatatcattcgggtttcgagacggccctga

SEQ ID NO: 638 - DNA, 236 bp, artificial
  oligo
  misc_feature (93)..(95): n is a, c, g, or t
  misc_feature (141)..(142): n is a, c, g, or t
gaccgcgtctcacaccggtataatagacgccgacgcagtttggagaccgtttaaacggtctctaccttgcgtcaatcgcagcgtctagtttgnnngcaggtgcaatgcaccccaagacatcgatcgtgcattgcacctgcnntatgcagcgaggttcgccgtcgtgatgtttgaagagcgtttaaacgctcttcgcccgcgagaaccggcattggattgtttcgagacggccctga

SEQ ID NO: 639 - DNA, 236 bp, artificial
  oligo
  misc_feature (93)..(95): n is a, c, g, or t
  misc_feature (141)..(142): n is a, c, g, or t
gaccgcgtctcacaccgagttgcttcgttcgaccagtgtttggagaccgtttaaacggtctctaccttatccgctcgtccgaacacggtttgnnngcaggtgcaatgccatctctagacggcaacctacattgcacctgcnntatgcactgtgggttaacgtcgcggtgtttgaagagcgtttaaacgctcttcgccctaccccgcctaattaccggtgtttcgagacggccctga

SEQ ID NO: 640 - DNA, 236 bp, artificial
  oligo
  misc_feature (93)..(95): n is a, c, g, or t
  misc_feature (141)..(142): n is a, c, g, or t
gaccgcgtctcacaccggcgcgccagatcgtaattaagtttggagaccgtttaaacggtctctacctctcgatatcaaccccgtaacgtttgnnngcaggtgcaatggtccatgtatagacccactgccattgcacctgcnntatgcatcgtttggccagacgcgtcagtttgaagagcgtttaaacgctcttcgccccgccgaatgaaaacgccggcgtttcgagacggccctga

SEQ ID NO: 641 - DNA, 236 bp, artificial
  oligo
  misc_feature (93)..(95): n is a, c, g, or t
  misc_feature (141)..(142): n is a, c, g, or t
gaccgcgtctcacaccgcacgcaactgtcgtaacacggtttggagaccgtttaaacggtctctacctcggaccggaagcgtctttcggtttgnnngcaggtgcaatgtcacttgacgtacagaccgaccattgcacctgcnntatgcataagcgacgccgatcataccgtttgaagagcgtttaaacgctcttcgccccgcactcgacctacgataccgtttcgagacggccctga

SEQ ID NO: 642 - DNA, 236 bp, artificial
  oligo
  misc_feature (93)..(95): n is a, c, g, or t
  misc_feature (141)..(142): n is a, c, g, or t
gaccgcgtctcacaccggttgtgtcgcatcgacgagtgtttggagaccgtttaaacggtctctacctacctcgatacacgcgcgctagtttgnnngcaggtgcaatgcgcacggtgacaccactctatcattgcacctgcnntatgcatgcgcgttgtcgattaccgggtttgaagagcgtttaaacgctcttcgcccctacgacgccccgtgctatcgtttcgagacggccctga

SEQ ID NO: 643 - DNA, 236 bp, artificial
  oligo
  misc_feature (93)..(95): n is a, c, g, or t
  misc_feature (141)..(142): n is a, c, g, or t
gaccgcgtctcacaccgacatatccgcggcgactcgggtttggagaccgtttaaacggtctctacctctcgtagttcaaacgacgtcgtttgnnngcaggtgcaatgctgccccttactgaatgacaccattgcacctgcnntatgcagataccgaaacgatgtcgtcgtttgaagagcgtttaaacgctcttcgccccgagattacggatcgcgagagtttcgagacggccctga

SEQ ID NO: 644 - DNA, 236 bp, artificial
  oligo
  misc_feature (93)..(95): n is a, c, g, or t
  misc_feature (141)..(142): n is a, c, g, or t
gaccgcgtctcacaccgcggcgaaagcgccgattagagtttggagaccgtttaaacggtctctacctcgggtgcgtttactcgtctagtttgnnngcaggtgcaatgctctgtctaccgctgcggtatcattgcacctgcnntatgcaactcgcgctaatcgctaagagtttgaagagcgtttaaacgctcttcgcccgacatatacgcgatacgacagtttcgagacggccctga

SEQ ID NO: 645 - DNA, 236 bp, artificial
  oligo
  misc_feature (93)..(95): n is a, c, g, or t
  misc_feature (141)..(142): n is a, c, g, or t
gaccgcgtctcacaccggaatttacggcggtcaccgggtttggagaccgtttaaacggtctctacctggcgtatccccgtacgctacgtttgnnngcaggtgcaatgtgcagtaccctaaccagtgcacattgcacctgcnntatgcagtcatgatcggaccgttcgagtttgaagagcgtttaaacgctcttcgccccgggtaagttgcgctaccgtgtttcgagacggccctga

SEQ ID NO: 646 - DNA, 236 bp, artificial
  oligo
  misc_feature (93)..(95): n is a, c, g, or t
  misc_feature (141)..(142): n is a, c, g, or t
gaccgcgtctcacaccgacgtcaaaccggtcgcttaggtttggagaccgtttaaacggtctctacctaacgatcgagcggtactctcgtttgnnngcaggtgcaatggccagaaggacattacatcgccattgcacctgcnntatgcaatatgctagcgtcgcattcggtttgaagagcgtttaaacgctcttcgcccaaggctcgatagttcgcgacgtttcgagacggccctga

SEQ ID NO: 647 - DNA, 236 bp, artificial
  oligo
  misc_feature (93)..(95): n is a, c, g, or t
  misc_feature (141)..(142): n is a, c, g, or t
gaccgcgtctcacaccgtaatgtctaacgctcgcgtcgtttggagaccgtttaaacggtctctacctcgatcgtgtggctcgtcgaggtttgnnngcaggtgcaatgtaatctgacacacccgcgagtcattgcacctgcnntatgcatagcatgcgctttgcggcgagtttgaagagcgtttaaacgctcttcgcccgtacgtttgtaactcggtaggtttcgagacggccctga

SEQ ID NO: 648 - DNA, 236 bp, artificial
  oligo
  misc_feature (93)..(95): n is a, c, g, or t
  misc_feature (141)..(142): n is a, c, g, or t
gaccgcgtctcacaccgactatgcggtaggtacgccggtttggagaccgtttaaacggtctctacctcagcgtaggtaatcgttgacgtttgnnngcaggtgcaatgggcgcacttggttcggctaatcattgcacctgcnntatgcataaactatgatcgcggacgtgtttgaagagcgtttaaacgctcttcgcccccgttggtgcgccaatgccggtttcgagacggccctga

SEQ ID NO: 649 - DNA, 236 bp, artificial
  oligo
  misc_feature (93)..(95): n is a, c, g, or t
  misc_feature (141)..(142): n is a, c, g, or t
gaccgcgtctcacaccgtcgattggccactaacggttgtttggagaccgtttaaacggtctctacctctcggccggatcacgacaaagtttgnnngcaggtgcaatgcgaccagcatgctatcaatgccattgcacctgcnntatgcacgttattccgagactacgtcgtttgaagagcgtttaaacgctcttcgcccttagagtacgcgataggctcgtttcgagacggccctga

SEQ ID NO: 650 - DNA, 236 bp, artificial
  oligo
  misc_feature (93)..(95): n is a, c, g, or t
  misc_feature (141)..(142): n is a, c, g, or t
gaccgcgtctcacaccgggccggacgaattacgataggtttggagaccgtttaaacggtctctaccttcgcctaaccgtattcgttcgtttgnnngcaggtgcaatgcagcacggacaagatcaactgcattgcacctgcnntatgcattacgcagcgacgtcttaatgtttgaagagcgtttaaacgctcttcgccctagttcgttcgacggagcgtgtttcgagacggccctga

SEQ ID NO: 651 - DNA, 236 bp, artificial
  oligo
  misc_feature (93)..(95): n is a, c, g, or t
  misc_feature (141)..(142): n is a, c, g, or t
gaccgcgtctcacaccggctactaccgcgtactacgcgtttggagaccgtttaaacggtctctacctacatcttaggttatgcggcggtttgnnngcaggtgcaatgccaagaatggatatccccagccattgcacctgcnntatgcagtacatcgccccggtcgattgtttgaagagcgtttaaacgctcttcgccccggtcgactggtcgatcattgtttcgagacggccctga

SEQ ID NO: 652 - DNA, 236 bp, artificial
  oligo
  misc_feature (93)..(95): n is a, c, g, or t
  misc_feature (141)..(142): n is a, c, g, or t
gaccgcgtctcacaccgcgcccgaagtcggttagtctgtttggagaccgtttaaacggtctctacctccgtcgtcgtgtgcagttgcgtttgnnngcaggtgcaatgcgaccgatctaccggaaatgtcattgcacctgcnntatgcaatacgaaagcgacgcgtatcgtttgaagagcgtttaaacgctcttcgcccgacttcactagagcgccgtagtttcgagacggccctga

SEQ ID NO: 653 - DNA, 236 bp, artificial
  oligo
  misc_feature (93)..(95): n is a, c, g, or t
  misc_feature (141)..(142): n is a, c, g, or t
gaccgcgtctcacaccggtatgtccgatccgcggtatgtttggagaccgtttaaacggtctctacctagcgcgacccgacgagaaacgtttgnnngcaggtgcaatggacctctacaatacagtgccccattgcacctgcnntatgcaaaaccgcgacgcactcctaggtttgaagagcgtttaaacgctcttcgcccaccgaccaaattccccggcggtttcgagacggccctga

SEQ ID NO: 654 - DNA, 236 bp, artificial
  oligo
  misc_feature (93)..(95): n is a, c, g, or t
  misc_feature (141)..(142): n is a, c, g, or t
gaccgcgtctcacaccggtgcgggcgttaaccagattgtttggagaccgtttaaacggtctctacctgcccgcagcaatcgtacgcggtttgnnngcaggtgcaatgattgcaccgacgtacacgtgacattgcacctgcnntatgcaccgtaatataccgtcctgtcgtttgaagagcgtttaaacgctcttcgccccgtgttaggtcgccgagcttgtttcgagacggccctga

SEQ ID NO: 655 - DNA, 236 bp, artificial
  oligo
  misc_feature (93)..(95): n is a, c, g, or t
  misc_feature (141)..(142): n is a, c, g, or t
gaccgcgtctcacaccggcgcatctaggcgttaccccgtttggagaccgtttaaacggtctctacctagaacgtcgtgtccgtaccagtttgnnngcaggtgcaatggtccatgaagcagcaattcggcattgcacctgcnntatgcaacgtccgtcggtatggacaagtttgaagagcgtttaaacgctcttcgccccgggaagcctattcgccgcggtttcgagacggccctga

SEQ ID NO: 656 - DNA, 236 bp, artificial
  oligo
  misc_feature (93)..(95): n is a, c, g, or t
  misc_feature (141)..(142): n is a, c, g, or t
gaccgcgtctcacaccgcgttcagtcgttagggtacggtttggagaccgtttaaacggtctctacctgagcgacaacgggcgataccgtttgnnngcaggtgcaatgtccgcacaaccctaaggtaagcattgcacctgcnntatgcagcgggcggtaacaagcgcgagtttgaagagcgtttaaacgctcttcgcccacgactgcgttttcgactgggtttcgagacggccctga

SEQ ID NO: 657 - DNA, 236 bp, artificial
  oligo
  misc_feature (93)..(95): n is a, c, g, or t
  misc_feature (141)..(142): n is a, c, g, or t
gaccgcgtctcacaccgccgcgcgtaaggtttcgtgcgtttggagaccgtttaaacggtctctacctcacgctatcccgtgaatgatgtttgnnngcaggtgcaatgcacactcggaactcactgcctcattgcacctgcnntatgcacgacttcgatttgtactccggtttgaagagcgtttaaacgctcttcgcccgccgattatcggtaattccggtttcgagacggccctga

SEQ ID NO: 658 - DNA, 236 bp, artificial
  oligo
  misc_feature (93)..(95): n is a, c, g, or t
  misc_feature (141)..(142): n is a, c, g, or t
gaccgcgtctcacaccgggtacgtgtcatatacgtcggtttggagaccgtttaaacggtctctacctagcggcgtaagacgattatcgtttgnnngcaggtgcaatgaccgatgtaaagctcaagccgcattgcacctgcnntatgcatatcgattcgccccgcctgtgtttgaagagcgtttaaacgctcttcgcccccggacgcggcgtatgatacgtttcgagacggccctga

SEQ ID NO: 659 - DNA, 236 bp, artificial
  oligo
  misc_feature (93)..(95): n is a, c, g, or t
  misc_feature (141)..(142): n is a, c, g, or t
gaccgcgtctcacaccgttcgcgcaacttcgtcgcaggtttggagaccgtttaaacggtctctaccttcgatcacgcgacggccccggtttgnnngcaggtgcaatggcgtaaaccgctacagccatacattgcacctgcnntatgcaccgcatcgtaggtgtcgaaagtttgaagagcgtttaaacgctcttcgcccttgatcgggttcgtcggagcgtttcgagacggccctga

SEQ ID NO: 660 - DNA, 236 bp, artificial
  oligo
  misc_feature (93)..(95): n is a, c, g, or t
  misc_feature (141)..(142): n is a, c, g, or t
gaccgcgtctcacaccgacgagcgatagcctaggacggtttggagaccgtttaaacggtctctacctccccgcgtattacgagatctgtttgnnngcaggtgcaatgcttacaggcaaaggctactcccattgcacctgcnntatgcacgtccgggtttagcattcgagtttgaagagcgtttaaacgctcttcgcccctcgacgcaaccgcagatcggtttcgagacggccctga

SEQ ID NO: 661 - DNA, 236 bp, artificial
  oligo
  misc_feature (93)..(95): n is a, c, g, or t
  misc_feature (141)..(142): n is a, c, g, or t
gaccgcgtctcacaccgtacactacgggaactcgccggtttggagaccgtttaaacggtctctacctgcggaaccatcgtcagtacggtttgnnngcaggtgcaatgccacggaagaaactgacgttccattgcacctgcnntatgcaccgttccgagtagccgtgtagtttgaagagcgtttaaacgctcttcgcccacgagtctggacgctcgacggtttcgagacggccctga

SEQ ID NO: 662 - DNA, 236 bp, artificial
  oligo
  misc_feature (93)..(95): n is a, c, g, or t
  misc_feature (141)..(142): n is a, c, g, or t
gaccgcgtctcacaccgtttcgcgatgcgcacgtattgtttggagaccgtttaaacggtctctacctactcgttcgaccgcgtcgcggtttgnnngcaggtgcaatgtcaacaaccagtacccggcagcattgcacctgcnntatgcatcaacgtagcgctattatcggtttgaagagcgtttaaacgctcttcgcccggtttgcgctaacgtgtggcgtttcgagacggccctga

SEQ ID NO: 663 - DNA, 236 bp, artificial
  oligo
  misc_feature (93)..(95): n is a, c, g, or t
  misc_feature (141)..(142): n is a, c, g, or t
gaccgcgtctcacaccgcccccgtcggacgcgtatgagtttggagaccgtttaaacggtctctacctagcgcacgttagacaccgcggtttgnnngcaggtgcaatgagcttccccactacatcagtgcattgcacctgcnntatgcaaccgttcgtacgaccaatccgtttgaagagcgtttaaacgctcttcgcccagtcgatcgcactcgacctagtttcgagacggccctga

SEQ ID NO: 664 - DNA, 236 bp, artificial
  oligo
  misc_feature (93)..(95): n is a, c, g, or t
  misc_feature (141)..(142): n is a, c, g, or t
gaccgcgtctcacaccgcggtagttgcgtagtagacggtttggagaccgtttaaacggtctctacctaattattagattcgcgcgtcgtttgnnngcaggtgcaatgcataaacgagcagacctcacccattgcacctgcnntatgcacggggcatagctcgtcggacgtttgaagagcgtttaaacgctcttcgcccaagcacgtaatccgtcctatgtttcgagacggccctga

SEQ ID NO: 665 - DNA, 236 bp, artificial
  oligo
  misc_feature (93)..(95): n is a, c, g, or t
  misc_feature (141)..(142): n is a, c, g, or t
gaccgcgtctcacaccggctgtacgaccgcgataagcgtttggagaccgtttaaacggtctctacctattcaacgacgaccgtgcgagtttgnnngcaggtgcaatgtgaagatagccacatcagcgccattgcacctgcnntatgcattaccgcatccgcttcgagcgtttgaagagcgtttaaacgctcttcgccccgcgcgccgagggctgtattgtttcgagacggccctga

SEQ ID NO: 666 - DNA, 236 bp, artificial
  oligo
  misc_feature (93)..(95): n is a, c, g, or t
  misc_feature (141)..(142): n is a, c, g, or t
gaccgcgtctcacaccgacggcgaacggctagtcgccgtttggagaccgtttaaacggtctctaccttacgggaccgatccggcaaagtttgnnngcaggtgcaatgcagtgcaccaatcagagttggcattgcacctgcnntatgcaccacgccgaacgccgtcagtgtttgaagagcgtttaaacgctcttcgcccccggctgtcgacgccagtacgtttcgagacggccctga

SEQ ID NO: 667 - DNA, 236 bp, artificial
  oligo
  misc_feature (93)..(95): n is a, c, g, or t
  misc_feature (141)..(142): n is a, c, g, or t
gaccgcgtctcacaccgaaccgggttacgggtctacggtttggagaccgtttaaacggtctctacctggcgtcgtacaatctcgagcgtttgnnngcaggtgcaatgcaaagcaccctgttagtcacccattgcacctgcnntatgcagcccatagcgacggtgatcggtttgaagagcgtttaaacgctcttcgccctgatgtaagctcgtatcgaagtttcgagacggccctga

SEQ ID NO: 668 - DNA, 236 bp, artificial
  oligo
  misc_feature (93)..(95): n is a, c, g, or t
  misc_feature (141)..(142): n is a, c, g, or t
gaccgcgtctcacaccggtagccggcccgacgcaaacgtttggagaccgtttaaacggtctctacctctcgatcgtgcggtatacgtgtttgnnngcaggtgcaatgctactagacagactacagcggcattgcacctgcnntatgcaggttcgacagcgttaggcccgtttgaagagcgtttaaacgctcttcgccctaccctcggcattcgaccgagtttcgagacggccctga

SEQ ID NO: 669 - DNA, 236 bp, artificial
  oligo
  misc_feature (93)..(95): n is a, c, g, or t
  misc_feature (141)..(142): n is a, c, g, or t
gaccgcgtctcacaccggttcgaacgcagaatgtacggtttggagaccgtttaaacggtctctacctttggggcgtagtcgtccgctgtttgnnngcaggtgcaatggccattcagtaactcccgatgcattgcacctgcnntatgcaagtacgcgcgaacaagtattgtttgaagagcgtttaaacgctcttcgccctcgcgtcgatggtcgagatagtttcgagacggccctga

SEQ ID NO: 670 - DNA, 236 bp, artificial
  oligo
  misc_feature (93)..(95): n is a, c, g, or t
  misc_feature (141)..(142): n is a, c, g, or t
gaccgcgtctcacaccgcacacgttatgttcgcgcgtgtttggagaccgtttaaacggtctctacctcgaccggccacatcgactaggtttgnnngcaggtgcaatggttcgccaaaccatccctttgcattgcacctgcnntatgcagattcatttccgcgccgtatgtttgaagagcgtttaaacgctcttcgccccgtgccggctcatcgttcgagtttcgagacggccctga

SEQ ID NO: 671 - DNA, 236 bp, artificial
  oligo
  misc_feature (93)..(95): n is a, c, g, or t
  misc_feature (141)..(142): n is a, c, g, or t
gaccgcgtctcacaccgcccgcgacaaatctgccgtagtttggagaccgtttaaacggtctctacctcgtggcccgtttagcgtgtcgtttgnnngcaggtgcaatgcactttccccttacctgcctgcattgcacctgcnntatgcactcgtcgcatagcgggtgaggtttgaagagcgtttaaacgctcttcgcccccgcgttgcacttgaccggggtttcgagacggccctga

SEQ ID NO: 672 - DNA, 236 bp, artificial
  oligo
  misc_feature (93)..(95): n is a, c, g, or t
  misc_feature (141)..(142): n is a, c, g, or t
gaccgcgtctcacaccgcgatcaaacgctgagtggacgtttggagaccgtttaaacggtctctacctcgttagtgtgtcgcgcataagtttgnnngcaggtgcaatgccacctttcgaatatggcacccattgcacctgcnntatgcaatcgcaccgcgaataacgcagtttgaagagcgtttaaacgctcttcgcccagtcctacgacgcgccgcaagtttcgagacggccctga

SEQ ID NO: 673 - DNA, 236 bp, artificial
  oligo
  misc_feature (93)..(95): n is a, c, g, or t
  misc_feature (141)..(142): n is a, c, g, or t
gaccgcgtctcacaccgagttgcacccgcggttaacggtttggagaccgtttaaacggtctctacctctttaccgcgcgacggacaagtttgnnngcaggtgcaatggaatcccccaattagcaagcgcattgcacctgcnntatgcacacctaactacgcccgcgacgtttgaagagcgtttaaacgctcttcgcccatcatcgttccgcgtcaaccgtttcgagacggccctga

SEQ ID NO: 674 - DNA, 236 bp, artificial
  oligo
  misc_feature (93)..(95): n is a, c, g, or t
  misc_feature (141)..(142): n is a, c, g, or t
gaccgcgtctcacaccgtcgctaacggttggaccgtggtttggagaccgtttaaacggtctctacctcgtcggtcacgagtacatgcgtttgnnngcaggtgcaatgctagacgctcataccgacgaacattgcacctgcnntatgcacttaccgtattaggcacgctgtttgaagagcgtttaaacgctcttcgccccgcgacatgatggtacgcgtgtttcgagacggccctga

SEQ ID NO: 675 - DNA, 236 bp, artificial
  oligo
  misc_feature (93)..(95): n is a, c, g, or t
  misc_feature (141)..(142): n is a, c, g, or t
gaccgcgtctcacaccgccttcgcgctatcttatgcggtttggagaccgtttaaacggtctctacctatctacgtaccgttagcggagtttgnnngcaggtgcaatgtcccctaaggcacacccgaatcattgcacctgcnntatgcactcgacgcgaaatttatctagtttgaagagcgtttaaacgctcttcgcccggtttaataccggcgcgtcagtttcgagacggccctga

SEQ ID NO: 676 - DNA, 236 bp, artificial
  oligo
  misc_feature (93)..(95): n is a, c, g, or t
  misc_feature (141)..(142): n is a, c, g, or t
gaccgcgtctcacaccgtcgcgataggcggtatctgagtttggagaccgtttaaacggtctctacctacttgcgctgtatcgcgccggtttgnnngcaggtgcaatgcacagcgcacgatctttggaacattgcacctgcnntatgcacgtggcgaatcgcggctaaagtttgaagagcgtttaaacgctcttcgcccccgtggatcgtttccgcactgtttcgagacggccctga

SEQ ID NO: 677 - DNA, 236 bp, artificial
  oligo
  misc_feature (93)..(95): n is a, c, g, or t
  misc_feature (141)..(142): n is a, c, g, or t
gaccgcgtctcacaccgttaatcctcggacgcggcgagtttggagaccgtttaaacggtctctacctaactcgcttcgtcggatatagtttgnnngcaggtgcaatgcgatccctccataccagtttgcattgcacctgcnntatgcaacgtatctcgtccaatcgcagtttgaagagcgtttaaacgctcttcgccctggaacggtcgacttcgggcgtttcgagacggccctga

SEQ ID NO: 678 - DNA, 236 bp, artificial
  oligo
  misc_feature (93)..(95): n is a, c, g, or t
  misc_feature (141)..(142): n is a, c, g, or t
gaccgcgtctcacaccgtgatgattcggcttgggcgcgtttggagaccgtttaaacggtctctacctgagcgtcgtcgcacactagtgtttgnnngcaggtgcaatgacgaccagtgcaactcaaagccattgcacctgcnntatgcagatagcgggtcgaactgctcgtttgaagagcgtttaaacgctcttcgcccccgaacctagtaccgcgttagtttcgagacggccctga

SEQ ID NO: 679 - DNA, 236 bp, artificial
  oligo
  misc_feature (93)..(95): n is a, c, g, or t
  misc_feature (141)..(142): n is a, c, g, or t
gaccgcgtctcacaccggctcgcgcattcttacgtcggtttggagaccgtttaaacggtctctacctagtctcgaccggttatccgtgtttgnnngcaggtgcaatgcaggaacacatcggtcaactgcattgcacctgcnntatgcatagacgtaggtacgccggtcgtttgaagagcgtttaaacgctcttcgcccatccactcgcgacgcctaatgtttcgagacggccctga

SEQ ID NO: 680 - DNA, 236 bp, artificial
  oligo
  misc_feature (93)..(95): n is a, c, g, or t
  misc_feature (141)..(142): n is a, c, g, or t
gaccgcgtctcacaccggattttcgaccccgaagcgtgtttggagaccgtttaaacggtctctacctcgttcgagccgcgtatccgtgtttgnnngcaggtgcaatggacaccagactatgatgcacccattgcacctgcnntatgcaattacgtttaatggcgcgtcgtttgaagagcgtttaaacgctcttcgccccatatcggcgcggcgcatttgtttcgagacggccctga

SEQ ID NO: 681 - DNA, 236 bp, artificial
  oligo
  misc_feature (93)..(95): n is a, c, g, or t
  misc_feature (141)..(142): n is a, c, g, or t
gaccgcgtctcacaccgtgcgggtatccgtaggacgcgtttggagaccgtttaaacggtctctaccttccgtaggatctcgacgccagtttgnnngcaggtgcaatgccagcgtacttagccgatagacattgcacctgcnntatgcagattacgtaatgtcgcgggcgtttgaagagcgtttaaacgctcttcgcccaattcgcgcgaattggcggggtttcgagacggccctga

SEQ ID NO: 682 - DNA, 236 bp, artificial
  oligo
  misc_feature (93)..(95): n is a, c, g, or t
  misc_feature (141)..(142): n is a, c, g, or t
gaccgcgtctcacaccgcggctatacggtgtagcaacgtttggagaccgtttaaacggtctctacctgtacaggccgacttcgcgacgtttgnnngcaggtgcaatgcacgtcccgtctaaaaagccacattgcacctgcnntatgcacggtccgatatccgccgggggtttgaagagcgtttaaacgctcttcgcccgtcacccggcggacttaagagtttcgagacggccctga

SEQ ID NO: 683 - DNA, 236 bp, artificial
  oligo
  misc_feature (93)..(95): n is a, c, g, or t
  misc_feature (141)..(142): n is a, c, g, or t
gaccgcgtctcacaccgatacggctaatcgaggcggagtttggagaccgtttaaacggtctctacctgcgccgaaactacaccgagggtttgnnngcaggtgcaatgcgcacgatcagaacacttaggcattgcacctgcnntatgcatctatcatatcgcacgtggtgtttgaagagcgtttaaacgctcttcgccctgcgcggacgctggtagcgagtttcgagacggccctga

SEQ ID NO: 684 - DNA, 236 bp, artificial
  oligo
  misc_feature (93)..(95): n is a, c, g, or t
  misc_feature (141)..(142): n is a, c, g, or t
gaccgcgtctcacaccgaacggcggacgacctacaatgtttggagaccgtttaaacggtctctacctgtacgtctagatgtcgatcggtttgnnngcaggtgcaatggcgaacaagagcacacgggaacattgcacctgcnntatgcatcgacagactatctgccgtagtttgaagagcgtttaaacgctcttcgccccactacgcgggttactagcggtttcgagacggccctga

SEQ ID NO: 685 - DNA, 236 bp, artificial
  oligo
  misc_feature (93)..(95): n is a, c, g, or t
  misc_feature (141)..(142): n is a, c, g, or t
gaccgcgtctcacaccgcgtttaagttggtaccccgcgtttggagaccgtttaaacggtctctaccttatggccgatgcaacgcaacgtttgnnngcaggtgcaatgccgcgaaccgaacagatgatccattgcacctgcnntatgcagttcattgggtcggcgtcgtgtttgaagagcgtttaaacgctcttcgcccccgggtattcgtgtggcgtcgtttcgagacggccctga

SEQ ID NO: 686 - DNA, 236 bp, artificial
  oligo
  misc_feature (93)..(95): n is a, c, g, or t
  misc_feature (141)..(142): n is a, c, g, or t
gaccgcgtctcacaccggcgtcaacggggatgaccgcgtttggagaccgtttaaacggtctctaccttgcgcgacccaacgatctcagtttgnnngcaggtgcaatgctaccactcgtccagctctatcattgcacctgcnntatgcatatgatccctaccaagcgcggtttgaagagcgtttaaacgctcttcgccccgcggaaatcgagcatcacggtttcgagacggccctga

SEQ ID NO: 687 - DNA, 236 bp, artificial
  oligo
  misc_feature (93)..(95): n is a, c, g, or t
  misc_feature (141)..(142): n is a, c, g, or t
gaccgcgtctcacaccggtgcgtggtcggatcgatatgtttggagaccgtttaaacggtctctacctgcccgacgattcgtcgcttggtttgnnngcaggtgcaatgcccttaccacgaaagtaaccccattgcacctgcnntatgcagtccccgcggtaacacgacggtttgaagagcgtttaaacgctcttcgcccagcggatgcgagcacggtatgtttcgagacggccctga

SEQ ID NO: 688 - DNA, 236 bp, artificial
  oligo
  misc_feature (93)..(95): n is a, c, g, or t
  misc_feature (141)..(142): n is a, c, g, or t
gaccgcgtctcacaccggcgccgaaaaacgtcggttggtttggagaccgtttaaacggtctctacctctataccgatacggagtgacgtttgnnngcaggtgcaatggcccctttatatcaaagcccgcattgcacctgcnntatgcatcgaaatcgacacgtcgcgagtttgaagagcgtttaaacgctcttcgcccccgcgagtcctggacataaggtttcgagacggccctga

SEQ ID NO: 689 - DNA, 236 bp, artificial
  oligo
  misc_feature (93)..(95): n is a, c, g, or t
  misc_feature (141)..(142): n is a, c, g, or t
gaccgcgtctcacaccgcgtacaaacgcgggagatccgtttggagaccgtttaaacggtctctacctatcccgtggcgatgtatcgagtttgnnngcaggtgcaatggccaccaagttcttcccaaggcattgcacctgcnntatgcacccctcgccatacgcgtaaggtttgaagagcgtttaaacgctcttcgccctgaacacgatacgcgcacgcgtttcgagacggccctga

SEQ ID NO: 690 - DNA, 236 bp, artificial
  oligo
  misc_feature (93)..(95): n is a, c, g, or t
  misc_feature (141)..(142): n is a, c, g, or t
gaccgcgtctcacaccgcggtattggacgaacgcgctgtttggagaccgtttaaacggtctctaccttgagtcgcgatatatgccgagtttgnnngcaggtgcaatgtgacacacgcgacctcctatccattgcacctgcnntatgcacgacgacctacaatggcggtgtttgaagagcgtttaaacgctcttcgccccgtccggtctatcgactaccgtttcgagacggccctga

SEQ ID NO: 691 - DNA, 236 bp, artificial
  oligo
  misc_feature (93)..(95): n is a, c, g, or t
  misc_feature (141)..(142): n is a, c, g, or t
gaccgcgtctcacaccgcgcgaataggtttgcgaatggtttggagaccgtttaaacggtctctacctttggtcccgcgaatcgcgttgtttgnnngcaggtgcaatggatgctcttagccacttacgccattgcacctgcnntatgcatcgtaagcggtcgaaaacgagtttgaagagcgtttaaacgctcttcgccccgaaattcccgacgctcccggtttcgagacggccctga

SEQ ID NO: 692 - DNA, 236 bp, artificial
  oligo
  misc_feature (93)..(95): n is a, c, g, or t
  misc_feature (141)..(142): n is a, c, g, or t
gaccgcgtctcacaccgacggcttgccaatccgtccggtttggagaccgtttaaacggtctctacctataagcgcggtatacgtaccgtttgnnngcaggtgcaatgtaccaagatctgtatcgccgccattgcacctgcnntatgcatcgcgaccccgatattcacggtttgaagagcgtttaaacgctcttcgccctagattggcgttcggtcgtcgtttcgagacggccctga

SEQ ID NO: 693 - DNA, 236 bp, artificial
  oligo
  misc_feature (93)..(95): n is a, c, g, or t
  misc_feature (141)..(142): n is a, c, g, or t
gaccgcgtctcacaccgtacgtcccgaagcgtccctcgtttggagaccgtttaaacggtctctaccttcgagtccgtaatttaacgtgtttgnnngcaggtgcaatgcacctgaccaaacgtcaccctcattgcacctgcnntatgcagtaaaccggcgccgacccgtgtttgaagagcgtttaaacgctcttcgcccgtatatgccgctacgcctttgtttcgagacggccctga

SEQ ID NO: 694 - DNA, 236 bp, artificial
  oligo
  misc_feature (93)..(95): n is a, c, g, or t
  misc_feature (141)..(142): n is a, c, g, or t
gaccgcgtctcacaccgcaccgcggctatcaaattcggtttggagaccgtttaaacggtctctacctgtctggtaaccgcgcgaacggtttgnnngcaggtgcaatgatcgcatattgtccacgaccgcattgcacctgcnntatgcagaatgatgcgccgcggtacagtttgaagagcgtttaaacgctcttcgccccgatttgatattaccgcgatgtttcgagacggccctga

SEQ ID NO: 695 - DNA, 236 bp, artificial
  oligo
  misc_feature (93)..(95): n is a, c, g, or t
  misc_feature (141)..(142): n is a, c, g, or t
gaccgcgtctcacaccgtcgtccgactcaacacgatggtttggagaccgtttaaacggtctctacctgagtcgtattaccacgctcggtttgnnngcaggtgcaatgctcatgagcgattactaccggcattgcacctgcnntatgcagtggacgatacatacaacgcgtttgaagagcgtttaaacgctcttcgcccttgatacgatctatccgggagtttcgagacggccctga

SEQ ID NO: 696 - DNA, 236 bp, artificial
  oligo
  misc_feature (93)..(95): n is a, c, g, or t
  misc_feature (141)..(142): n is a, c, g, or t
gaccgcgtctcacaccgcgtatcgtatctgacgtgtggtttggagaccgtttaaacggtctctacctagtgaacggttgggtacgccgtttgnnngcaggtgcaatgcgaagcaatcctagccgtatccattgcacctgcnntatgcaacgagtcgtcatcgtgccgtgtttgaagagcgtttaaacgctcttcgcccgaccgcaaccgtcgttcaaggtttcgagacggccctga

SEQ ID NO: 697 - DNA, 236 bp, artificial
  oligo
  misc_feature (93)..(95): n is a, c, g, or t
  misc_feature (141)..(142): n is a, c, g, or t
gaccgcgtctcacaccgccgatataatatcggggaaggtttggagaccgtttaaacggtctctacctattcgccgctaatcacggatgtttgnnngcaggtgcaatgggctatttgatccacccgatgcattgcacctgcnntatgcagcgtactactactaagcggtgtttgaagagcgtttaaacgctcttcgcccgttagcgtccgaaaacccgggtttcgagacggccctga

SEQ ID NO: 698 - DNA, 236 bp, artificial
  oligo
  misc_feature (93)..(95): n is a, c, g, or t
  misc_feature (141)..(142): n is a, c, g, or t
gaccgcgtctcacaccgtgtcgttcgatgactcggatgtttggagaccgtttaaacggtctctacctgcagcagtccgccgatttcggtttgnnngcaggtgcaatgcctattgaacccggaaatccgcattgcacctgcnntatgcaacgtatagtacgcttcgcacgtttgaagagcgtttaaacgctcttcgccctccggccgcattcccgtaccgtttcgagacggccctga

SEQ ID NO: 699 - DNA, 236 bp, artificial
  oligo
  misc_feature (93)..(95): n is a, c, g, or t
  misc_feature (141)..(142): n is a, c, g, or t
gaccgcgtctcacaccgccggatcgtgatcctcggttgtttggagaccgtttaaacggtctctaccttatcctcgcaatatacgcgagtttgnnngcaggtgcaatgacttcagtagatccccaggcccattgcacctgcnntatgcatcgtgccaagcactagtacggtttgaagagcgtttaaacgctcttcgcccgatcgcgttgataaggcgtcgtttcgagacggccctga

SEQ ID NO: 700 - DNA, 236 bp, artificial
  oligo
  misc_feature (93)..(95): n is a, c, g, or t
  misc_feature (141)..(142): n is a, c, g, or t
gaccgcgtctcacaccgtagcgaaatcgtacccgagcgtttggagaccgtttaaacggtctctacctgcgcgacacatgggcggttcgtttgnnngcaggtgcaatggcactttacgtccgcttactgcattgcacctgcnntatgcacatatttacgtggtcgtcgtgtttgaagagcgtttaaacgctcttcgcccgaccggcgagtccgtaccatgtttcgagacggccctga

SEQ ID NO: 701 - DNA, 236 bp, artificial
  oligo
  misc_feature (93)..(95): n is a, c, g, or t
  misc_feature (141)..(142): n is a, c, g, or t
gaccgcgtctcacaccgttatagcgtaataccgccgggtttggagaccgtttaaacggtctctacctgttaaactaaatcggcgcgcgtttgnnngcaggtgcaatgtcaaccaccacatgacggaagcattgcacctgcnntatgcagtaactacggcgcatcgtctgtttgaagagcgtttaaacgctcttcgcccgcgaacatgatcgctacgaggtttcgagacggccctga

SEQ ID NO: 702 - DNA, 236 bp, artificial
  oligo
  misc_feature (93)..(95): n is a, c, g, or t
  misc_feature (141)..(142): n is a, c, g, or t
gaccgcgtctcacaccgcgcgacatgcatcgctttcggtttggagaccgtttaaacggtctctacctagtcgaacccggcgcgattggtttgnnngcaggtgcaatggcacaccatcgacttccgtatcattgcacctgcnntatgcacgcggcgtgaacagaccgatgtttgaagagcgtttaaacgctcttcgcccagcgaatacgtacccgtgtagtttcgagacggccctga

SEQ ID NO: 703 - DNA, 236 bp, artificial
  oligo
  misc_feature (93)..(95): n is a, c, g, or t
  misc_feature (141)..(142): n is a, c, g, or t
gaccgcgtctcacaccgcgtcacggcgagcgaaccccgtttggagaccgtttaaacggtctctacctttattacgtgcgccggccgggtttgnnngcaggtgcaatgctaacagccgaaaagcccaagcattgcacctgcnntatgcacggtacacgacttcgcccgcgtttgaagagcgtttaaacgctcttcgccctaccgcggtcgtttagatcggtttcgagacggccctga

SEQ ID NO: 704 - DNA, 9550 bp, artificial
  PAC Vector
  misc_feature (7488)..(7488): n is a, c, g, or t
  misc_feature (7490)..(7491): n is a, c, g, or t
  misc_feature (7493)..(7493): n is a, c, g, or t
ttaagcttgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatcgcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtccctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggatagctagagccagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttataaatactgaataataagatgacatgaactactatacgtactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagagccagtacaagcaaaaagcagatcttgtcatcgttgggagtgaattagcccttccagtccccccttttcttttaaaaagtggctaagatctacagctgccttgtaagtcattggtcttaaagtcgacgcggggaggcggcccaaagggagatccgactcgtctgagggcgaaggcgaacacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgttgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacttaacgcgtggatccttatctgtgccccagtttgctagggaggtcgcagtatctggccacagccacctcgtgctgctcgacgtaggtttctttgtcggcctccttgattctttccagccttctgtccacatagtagacgccgggcatcttgaggttcttagcgggtttcttggatctgtatgtggttttcaagttgcagatcaggtggcccccgcccacgagcttcagggccatgtcggctctgccttccaggccgccgtcagcggggtacagggtttcggtggaggcctcccagccgagtgttttcttctgcatcacagggccgttggatgggaagttcacccctctgatcttgacgttgtagatgaggcagccgtcctggaggctggtgtcctgggtagcggtcagcacgcccccgtcctcgtatgtggtgactctctcccatgtgaagccctcggggaaggactgcttaaagaagtcggggatgccctgggtgtggttgatgaaggttttgctgccgtacatgaagctggtagccaggatgtcgaaggcgaaggggagagggccgccctcgaccgccttgattctcatggtctgggtgccctcgtagggcttgccttcgccctcggatgtgcacttgaagtggtggttgttcacggtgccctccatgtacagcttcatgtgcatgttctccttaatcagctcgctcacggggccgggattttcctccacgtccccgcatgttagcagacttcccctgccctcggcaccgggcttgcgggtcatgcaccaggtgcgcggtccttcgggcacctcgacgtcggcggtgacggtgaagccgagccgctcgtagaaggggaggttgcggggcgcggaggtttccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccacggcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggccgtacgtcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccaacgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggccatgtgcgctctgcccactgacgggcaccggagccaattcccactcctttcaagacctagaaggtccattagctgcaaagattcctctctgtttaaaactttatccatctttgcacccgggccccctcgagccggcgccaaagtggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctcaagatctagttacgccaagcttggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagcttgtccaactggtcgggtttaagagctatgctggaaacagcatagcaagtttaaataaggctagtccgttatcaacttgaaaaagtggcaccgagtcggtgctggttccatggtgtaatggttagcactctggactctgaatccagcgatccgagttcaaatctcggtggaacctagcacacaagcacgttacccgtttgagagctatgccgggansnnsnaaccggcatagcaagttcaaataaggctagtccgttatcaacttggcccggctttaaggccggtcctagcaacgggccaagtggcaccgagtcggtgctgcatgggtggttcagtggtagaattctcgcctgccacgcgggaggcccgggttcgattcccggcccatgcaggactgagagctgttgacacgtttgagagctatgctggagcccgaacagcatagcaagttcaaataaggctagtccgttatcaacttggcccgaaaaaaaagaaaaaaagaaaaaaagaaaaacgggccaagtggcaccgagtcggtgctttttttcaccgtggaatgcgagaattctgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttgctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccaacgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagagccgtttatgtatcgagctaggcacttaaatacaatatctctgcaatgcggcaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactaca

(19) United States
(12) Patent Application Publication
(10) Pub. No.: US 2024/0366667 A1
(43) Pub. Date: Nov. 7, 2024

(54) DISRUPTIONS OF PDCD1, ADORA2A, AND CTLA4 GENES AND USES THEREOF

(71) Applicant: WESTLAKE UNIVERSITY, Hangzhou (CN)

(72) Inventors: Zhike LU, Hangzhou (CN); Lijia MA, Hangzhou (CN); Ke NI, Hangzhou (CN)

(21) Appl. No.: 18/572,307

(22) PCT Filed: May 31, 2022
(86) PCT No.: PCT/CN2022/096249

§ 371 Date: Dec. 20, 2023

(30) Foreign Application Priority Data
Jun. 21, 2021 (CN) 202110686751.3

(51) Int. Cl. A61K 35/17; A61K 39/00; A61P 35/00; C12N 5/0783; C12N 9/22; C12N 15/11; C12N 15/86; C12N 15/87

(52) U.S. Cl. CPC A61K 35/17; A61K 39/4611; A61K 39/4631; A61K 39/464411; A61P 35/00; C12N 5/0636; C12N 9/22; C12N 15/111; C12N 15/86; C12N 15/87; A61K 2239/00; A61K 2239/11; A61K 2239/21; C12N 2310/20; C12N 2740/15043

(57) ABSTRACT

Provided are methods for disrupting Pdcd1, Adora2a, and Ctla4 genes using a Cas and guide RNAs targeting the three genes. Also provided are methods for treatment of cancers and/tumors by administering to subjects in need thereof engineered immune cells wherein the Pdcd1, Adora2a, and Ctla4 genes are disrupted in the engineered immune cells and wherein the engineered immune cells optionally further comprise a chimeric antigen receptor for targeting cancer or tumor cells.

### SEQUENCE LISTING

The instant application contains a Sequence Listing, which has been submitted electronically in ASCII format and is hereby incorporated by reference in its entirety.

### FIELD

The present disclosure relates to disruptions of Pdcd1, Adora2a, and Ctla4 genes and uses thereof.

### BACKGROUND

In many instances, disturbing a single gene is insufficient to direct an interested phenotype, even though that single gene does contribute to the phenotype. For example, sets of transcription factors crosstalk with one another to orchestrate an invasion-metastasis cascade in cancer progression (Hanahan, D. & Weinberg, R. A. Hallmarks of cancer: the next generation. Cell 144, 646-674, doi: 10.1016/j.cell.2011.02.013 (2011)).

Simultaneously disturbing multiple targets have unique advantages in identifying targets for desired outcomes where there are high-order gene interactions. A combined immunotherapy has been reported to promote anti-tumor immunity by targeting multiple immune repressors that work in complementary and nonredundant mechanisms (Postow, M. A. et al. Nivolumab and ipilimumab versus ipilimumab in untreated melanoma. N Engl J Med 372, 2006-2017, doi: 10.1056/NEJMoa1414428 (2015)). A study showed that antibodies against PD-L1 and CTLA-4 and a small molecule antagonist targeting A2AR (ADORA2A) induced anti-tumor responses in a mouse model (Willingham, S. B. et al. A2AR Antagonism with CPI-444 Induces Antitumor Responses and Augments Efficacy to Anti-PD-(L) 1 and Anti-CTLA-4 in Preclinical Models. Cancer Immunol Res 6, 1136-1149, doi: 10.1158/2326-6066.CIR-18-0056 (2018)). In the clinic, combined immune therapy has been considered a promising direction to overcoming resistance to cancer immunotherapy (Sharma, P., Hu-Lieskovan, S., Wargo, J. A. & Ribas, A. Primary, Adaptive, and Acquired Resistance to Cancer Immunotherapy. Cell 168, 707-723, doi: 10.1016/j.cell.2017.01.017 (2017); Khair, D. O. et al. Combining Immune Checkpoint Inhibitors: Established and Emerging Targets and Strategies to Improve Outcomes in Melanoma. Front Immunol 10, 453, doi: 10.3389/fimmu.2019.00453 (2019)).

The present disclosure provides methods of targeting three genes, namely, the Pdcd1, Adora2a, and Ctla4 genes and methods of treating diseases such as cancers by disrupting these genes.

### SUMMARY

In one aspect, the present disclosure provides a method for disrupting a Pdcd1 gene, an Adora2a gene, and an Ctla4 gene (collectively referred to as “PAC”) in a cell, comprising: introducing into a cell comprising a Cas (CRISPR-associated protein) (e.g., Cas9), a first guide RNA (gRNA) comprising a first spacer sequence targeting the Pdcd1 gene, a second gRNA comprising a second spacer sequence targeting the Adora2a gene, a third gRNA comprising a third spacer sequence targeting the Ctla4 gene, wherein the Pdcd1 gene, the Adora2a gene, and the Ctla4 are disrupted. In some embodiments, the method of disrupting PAC further comprises introducing into the cell a chimeric antigen receptor (CAR). In some embodiments, the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In one aspect, the present disclosure provides a method of treating a disease such as cancer or tumor in a subject in need thereof, comprising administering to the subject an engineered immune cell comprising: a Cas, a first gRNA comprising a first spacer sequence targeting a Pdcd1 gene, a second gRNA comprising a second spacer sequence targeting an Adora2a gene, a third gRNA comprising a third spacer sequence targeting an Ctla4 gene, wherein the Pdcd1 gene, the Adora2a gene, and the Ctla4 are disrupted. In some embodiments, the engineered immune cell comprises a chimeric antigen receptor (CAR). In some embodiments, the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In one aspect, the present disclosure provides a vector comprising one or more of nucleotide sequences wherein the nucleotide sequences are selected from: (a) a first nucleotide sequence encoding a first gRNA comprising a spacer sequence complementary with a first target domain of a Pdcd1 gene; (b) a second nucleotide sequence encoding a second gRNA comprising a second spacer sequence complementary with a second target domain of an Adora2a gene; (c) a third nucleotide sequence encoding a third gRNA comprising a third spacer sequence complementary with a third target domain of a Ctla4 gene; and (d) a fourth nucleotide sequence encoding a Cas. In some embodiments, the vector further comprises a nucleotide sequence encoding a CAR. In some embodiments, the present disclosure provides a vector comprising one of the first, second, third, and fourth nucleotide sequences. In some embodiments, the present disclosure provides a vector comprising any two of the first, second, third, and fourth nucleotide sequences. In some embodiments, the present disclosure provides a vector comprising any three of the first, second, third, and fourth nucleotide sequences. In some embodiments, the present disclosure provides a vector comprising the first, second, third, and fourth nucleotide sequences.

In one aspect, the present disclosure provides an engineered cell comprising: a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene. In some embodiments, the engineered cell further comprises a chimeric antigen receptor (CAR).

In one aspect, the present disclosure provides a pharmaceutical composition comprising a population of the engineered cells, e.g., engineered immune cells, as described herein.

In one aspect, the present disclosure provides a kit comprising a Cas, a first gRNA comprising a first spacer sequence targeting a Pdcd1 gene, a second gRNA comprising a second spacer sequence targeting a Adora2a gene, and a third gRNA comprising a third spacer sequence targeting a Ctla4 gene. In some embodiments, the kit further comprises a nucleotide sequence encoding a chimeric antigen receptor (CAR).

In one aspect, the present disclosure provides a kit comprising one or more vectors comprising one or more nucleotide sequences encoding a Cas, a first gRNA comprising a first spacer sequence targeting a Pdcd1 gene, a second gRNA comprising a second spacer sequence targeting an Adora2a gene, a third gRNA comprising a third spacer sequence targeting an Ctla4 gene, and/or a CAR.

In one aspect, the present disclosure provides an engineered immune cell for treating a disease such as cancer or tumor in a subject in need thereof, wherein the engineered immune cell comprises a Cas, a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene, wherein the Pdcd1 gene, the Adora2a gene, and the Ctla4 are disrupted.

In one aspect, the present disclosure provides use of an engineered immune cell for the manufacture of a medicament for treating a disease such as cancer or tumor, wherein the immune cell comprises a Cas, a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene, wherein the Pdcd1 gene, the Adora2a gene, and the Ctla4 are disrupted.

### BRIEF DESCRIPTION OF THE DRAWINGS

FIG. 1A to FIG. 1D illustrate in vivo screening and validation for combinatorial checkpoint blockades to boost T cells.

FIG. 1A. Schema of an in vivo screening for check point blockade. CD8+ T cells were collected from OT-I mice (dark grey), which were infected by a screening library and further injected into recipient mice (grey) inoculated with Hepa1-6 cells with stable H2Kb-OVA257-264 expression. (TIL: tumor-infiltrating lymphocytes.)

FIG. 1B. Ranks of the log2FC values of the engineered T cells across the CP, TCR, CS, and NC groups. More T cells from the CP group were enriched into the tumor samples compared to T cells from the groups of TCR, CS, and NC. (CP: Checkpoint group; TCR: T cell receptor group; CS: Co-stimulatory molecule group; NC: Negative control group.)

FIG. 1C. Validation of the PAC combinatory gene perturbation. Tumor size curves were plotted for the mice receiving OT-I CD8+ T cells with a combined Adora2a, Ctla4 and Pdcd1 disruption (PAC), a combined Ctla4 and Pdcd1 disruption (PCN), or only a Pdcd1 disruption (PNN), and the mice that did not receive a CD8+ T injection (CTL). The black arrow (timeline) indicated the day of tumor cell line inoculation; the triangles indicated d0, d21, and d42 after T cell injection. Tumor sizes were recorded every 3 days. The number of mice in each group was: 11 in PAC, 6 in PNN, 9 in PCN, and 12 in CTL.

FIG. 1D. In vivo imaging of mice. In vivo imaging for mice receiving OT-I CD8+ T cells with the PAC, PCN, or PNN disruption, and the mice that did not receive CD8+ T injection (CTL). The crosses (X) indicated dead mice or mice sacrificed because of tumor size limitation (≤4000 mm3).

FIG. 2A to FIG. 2C show establishment of H2Kb-OVA257-264 expression tumor cells and cytotoxicity of T cell against H2Kb-OVA257-264+ tumor cells in vitro.

FIG. 2A. Expression of H2Kb-OVA257-264 on tumor cell lines.

FIG. 2B. CD107a and CD8a expression in OT-I CD8+ T cells co-cultured with tumor cell lines with or without H2Kb-OVA257-264 expression.

FIG. 2C. PI and Annexin V staining in tumor cell lines co-cultured with OT-I CD8+ T cells.

FIG. 3 shows a histogram distribution of the log2FC values of engineered T cells. Only a small number of gRNA combinations showed positive log2FC, which indicated that most T cells were not capable of infiltrating into tumors.

FIG. 4 shows ranks of gRNA-combinations. The gRNA-combinations were ranked according to their enrichment in three batches of screen and six gRNA replicates in a designed multiplexed CRISPR library. The PAC combination (Adora2a, Ctla4, and Pdcd1) showed the most positive hits under different log2FC cutoffs. Only the gRNA combinations that showed at least 3 positive hits were plotted in FIG. 4.

FIG. 5 shows log2FC values of gRNA-combinations with PAC gene disruption. The distribution of log2FC of four different PAC-containing combinations were plotted as violin plots.

FIG. 6 illustrates knockout efficiencies of the gRNAs of the PAC-combination. FIG. 6 shows representative amplicon sequencing of the sgRNA target sites in Cas9+ OT1 CD8+ T cells at day 3 post lentivirus transduction.

FIG. 7 shows survival curves of mice in a validation experiment. Survival rate for the mice that received OT-I CD8+ T cells with a combined Adora2a, Ctla4, and Pdcd1 disruption (PAC), a combined Ctla4 and Pdcd1 disruption (PCN), or only a Pdcd1 disruption (PNN), and the mice that did not receive CD8+ T injection (CTL). The number of mice in each group was: 11 in PAC, 6 in PNN, 9 in PCN, and 12 in CTL.

FIG. 8 illustrates construction of a screening library. FIG. 8A is a schema of in-library ligation. Rectangles: spacer sequence of gRNA. FIG. 8B is a schema of construction of a plasmid library.

### DETAILED DESCRIPTION

All publications cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

Although any methods and materials similar or equivalent to those described herein may be used in the practice for testing of the present disclosure, exemplary materials and methods are described herein.

### Definitions

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

As used in this specification and the appended claims, the singular forms “a,” “an,” and “the” include plural referents unless the content clearly dictates otherwise. Thus, for example, reference to “a cell” includes one, two, or more cells, and the like.

The terms “polynucleotide” and “nucleic acid” are used interchangeably herein to refer to a polymeric form of nucleotides of any length, either ribonucleotides or deoxyribonucleotides. This term refers only to the primary structure of the molecule. A polynucleotide disclosed herein may be modified, e.g., with a labeling group such as a fluorophore, with a biotin, or with a phosphorothioate.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like. A peptide disclosed herein may be modified, e.g., with a labeling group such as a fluorophore, a biotin, a His tag, or phosphorothioate.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “subject” means any animal such as a mammal, e.g., a human.

The term “cancer antigen” or “tumor antigen” refers to any antigen that expresses specifically on a cancer or tumor cell. For example, cancer or tumor antigens include those antigens that have high, homogeneous expressions in cancers or tumors but not in healthy tissues. For instance, cancer or tumor antigens include Her 2, Claudine 18.2, CD19, BCMA, CD20, NYESO-1, MAGE-1, Tyrosinase, MUC1, CEA, Mam-A, hTERT, Sialyl-Tn, WT1,α-FetoProtein, CA-125, gp-100, p53, Rax, Src, AFP, PSA, and the like.

The term “chimeric antigen receptor” (CAR, also known as chimeric immunoreceptor, chimeric T cell receptor or artificial T cell receptor) refers to a cell-surface receptor comprising an extracellular antigen binding domain, a transmembrane domain, and one or more cytoplasmic co-stimulatory signaling domains not naturally found together on a single receptor protein. A CAR may comprise an extracellular hinge region (e.g., a flexible spacer) between the extracellular antigen binding domain and the transmembrane domain. A CAR may combine an antigen-binding domain, a hinge domain, a transmembrane domain, a co-stimulatory domain, and CD247 into a single receptor.

The term “immune cells” refers to cells of the immune system in animals, including neutrophils, eosinophils, basophils, mast cells, monocytes, macrophages, dendritic cells, natural killer cells, and lymphocytes (e.g., B cells and T cells). T cells comprise CD4 cells, CD8+ cells, gamma delta T cells (γδT cells), NK T cells and/or regulatory T cells (Treg).

The term “engineered cells” refer to cells that are genetically engineered, including the cells in which one or more genes are disrupted, and optionally engineered to express a CAR.

The term “engineered immune cells” refer to immune cells that are genetically engineered, including the cells in which one or more genes are disrupted, and optionally engineered to express a CAR.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

As used herein, “affinity” refers to the strength of the sum total of noncovalent interactions between a molecule or a molecular structure (e.g., a ligand) and its binding partner (e.g., a receptor). The affinity of a molecule for its partner can generally be represented by the equilibrium dissociation constant (K_{D}) (or its inverse equilibrium association constant, K_{A}). Affinity can be measured by common methods known in the art, including those described herein. See, for example, Pope M. E., Soste M. V., Eyford B. A., Anderson N. L., Pearson T. W., (2009) J. Immunol. Methods. 341 (1-2): 86-96 and methods described therein.

As used herein, the term “specifically bind” or “bind to,” or “recognizes” refers to binding that is measurably different from a non-specific interaction. For example, in some embodiments, a binding molecule, such as a single domain antibody, specifically binds to a target molecule, such as an antigen, when the binding molecule reacts or associates more frequently, more rapidly, with greater duration, and/or with greater affinity with the particular target molecule than it does with alternative molecules. A binding molecule “specifically binds” to an antigen if it binds with greater affinity, avidity, more readily, and/or with greater duration than it binds to other molecules. It is understood that a binding molecule that specifically binds to a first target may or may not specifically bind to a second target. As such, “specific binding” does not necessarily require (although it can include) exclusive binding. In some embodiments, specific binding can be determined, for example, by comparing binding of a particular antibody to binding of an antibody that does not bind to a particular antigen. Specific binding for a particular antigen can be shown, for example, when a binder partner has a K_{D} for an antigen of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, at least about 10⁻¹² M, or greater, where Kp refers to a dissociation rate of the a binder partner/antigen interaction. In some embodiments, a binder partner that specifically binds an antigen will have a K_{D} that is 20, 50, 100, 500, 1000, 5,000, 10,000 or more times greater than the K_{D} of a binder partner that does not bind to the same antigen. In some embodiments, the binding between a binder partner and a particular antigen can be shown by an EC50 value, determined using suitable methods known in the art, including, for example, flow cytometry assay.

As used herein, a “composition” refers to any mixture of two or more products, substances, or compounds, including but not limited to, proteins, antibodies, polynucleotides, vectors, or cells. It may be a solution, a suspension, liquid, powder, a paste, aqueous, non-aqueous, or any combination thereof.

As used herein, a “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. Some non-limiting examples of the components that could be included in the composition are carriers, stabilizers, diluents, dispersing agents, suspending agents, thickening agents, and/or excipients. The pharmaceutical composition facilitates administration of the engineered cells described herein to a subject. Multiple techniques of administration exist in the art including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of a therapeutic compound, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “effective amount” or “therapeutically effective amount” refers to an amount of a pharmaceutical composition which is sufficient to significantly and positively modify the symptoms and/or conditions to be treated (e.g., provide a positive clinical response). The effective amount of a pharmaceutical composition will vary with the particular condition being treated, the severity of the condition, the duration of treatment, the nature of concurrent therapy, the particular composition being employed, the particular pharmaceutically-acceptable excipient(s) and/or carrier(s) utilized, and like factors with the knowledge and expertise of the attending physician.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting or eliminating the disease or progression of the disease, inhibiting or slowing the disease or its progression, arresting its development, and remission (whether partial or total).

As used herein, the term “disrupting,” “disrupted,” “disruption,” “perturbing,” “perturbation,” “perturbed,” “knockout,” or knocked out” in the context of manipulating a gene, means that the gene is genetically engineered to be inoperative, inactive, or removed, or at least partially inoperative, inactive, or removed.

### Screening of Combinatory Gene Disruptions

The present disclosure provides a multiplexed gRNA library for in vivo screening of effective combinations of gene perturbations, e.g., for screening of boosted tumor-infiltrating T cells (TILs). For example, to investigate synergistic or additive anti-tumor efficacy, immune cells, such as T cells, can be genetically engineered with a combination of gRNAs targeting multiple genes simultaneously, e.g., with one, two, three, and four gRNAs simultaneously. The engineered immune cells, such as engineered T cells, can be screened for activities such as antitumor activity in a tumor environment.

In some embodiments, an in vivo screening library of gRNAs is provided to target multiple genes, such as six checkpoint genes, in T cells. In one embodiment, the in vivo screen library includes fifty-six combinations of gRNAs, including fifteen 4-gRNA combinations, twenty 3-gRNA combinations, fifteen 2-gRNA combinations, and six single-gRNA combinations, targeting 4 checkpoint genes, three checkpoint genes, 2 checkpoint genes, and one checkpoint gene, respectively. The gRNA combinations can be ranked according to their enrichment levels in tumor tissues. In one embodiment, T cells engineered with a 3-gRNA combination that simultaneously targets Pdcd1, Adora2a, and Ctla4 (denoted as “PAC” herein) are more enriched in a tumor environment than other gRNA combinations.

Validation experiments can be further performed to confirm the results from an in vivo screening. In one embodiment, T cells engineered with a candidate combination of gRNAs and a control combination of gRNAs may be administered to mice with a tumor. In one embodiment, T cells knocked out only at the Pdcd1 loci (denoted as “PNN”), at Pdcd1 and Ctla4 loci (denoted as “PCN”), as well as Pdcd1, Ctla4, and Adora2a loci (“PAC”) are obtained. For example, the T cells engineered with the PNN, PCN, and PAC gRNA combinations, respectively, are injected into the recipient mice inoculated with Hepa1-6 cancer cells expressing H2Kb-OVA₂₅₇₋₂₆₄. After the T cell therapy, the weight loss of the mice and the tumor size are monitored, and the efficacy of the gene perturbations is confirmed.

### Disruptions of Pdcd1, Adora2a, and Ctla4 Genes

In some embodiments, the present disclosure provides a method for disrupting a Pdcd1 gene, an Adora2a gene, and a Ctla4 gene (collectively referenced as “PAC”) in a cell, comprising: introducing into the cell a Cas (e.g., Cas9), a first gRNA comprising a first spacer sequence targeting the Pdcd1 gene, a second gRNA comprising a second spacer sequence targeting the Adora2a gene, and a third gRNA comprising a third spacer sequence targeting the Ctla4 gene, wherein the Pdcd1 gene, the Adora2a gene, and the Ctla4 are disrupted.

In some embodiments, the present disclosure provides a genetically engineered immune cell for treating a disease such as cancer or tumor in a subject in need thereof, wherein the immune cell comprises a Cas (e.g., Cas9), a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene, wherein the Pdcd1 gene, the Adora2a gene, and the Ctla4 are disrupted.

In some embodiments, the present disclosure provides use of a genetically engineered immune cell for the manufacture of a medicament for treating a cancer, wherein the immune cell comprises a Cas (e.g., Cas9), a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene, wherein the Pdcd1 gene, the Adora2a gene, and the Ctla4 are disrupted.

A Cas can be introduced into a cell in the form of a polynucleotide sequence (e.g., in a plasmid or mRNA) encoding the Cas protein or in the form of RNP. The gRNAs can be introduced into a cell in the form of a polynucleotide sequence encoding the gRNA or in the form of RNP.

In some embodiments, a Cas and a gRNA may be introduced into a cell via one or more vectors expressing the Cas and gRNA. In some embodiments, a gRNA can be delivered into cells in other forms, e.g., in the form of ribonucleoprotein (RNP), where a guide RNA is mixed with a Cas enzyme protein. RNPs can be delivered into cells via electroporation.

In some embodiments, the method for disrupting PAC comprises introducing into the cell one or more polynucleotides encoding the Cas, the first, second, and third gRNAs. For example, a single vector can be introduced into a cell, wherein the vector comprises a nucleotide sequence encoding the Cas, a nucleotide sequence encoding the first gRNA, a nucleotide sequence encoding the second gRNA, and a nucleotide sequence encoding the third gRNA. As another example, two vectors can be introduced into a cell, wherein one of the vectors comprises a nucleotide sequence encoding the Cas, and the other one of the vectors comprises a nucleotide sequence encoding the first gRNA, a nucleotide sequence encoding the second gRNA, and a nucleotide sequence encoding the third gRNA. As understood by a person of ordinary skill in the art, the Cas, the first gRNA, the second gRNA, and the third gRNA, can be encoded by a single vector, or encoded by two vectors or three vectors in any combination, or encoded by four vectors individually.

In some embodiments, the first, second, and third gRNAs are single-molecule guide RNAs (sgRNAs).

In some embodiments, the method for disrupting PAC is conducted in vitro. In some embodiments, the method for disrupting PAC is conducted in vivo, e.g., in a subject with cancer.

In some embodiments, the cell is an immune cell. In some embodiments, the cell is a lymphocyte cell. In some embodiments, the cell is a T cell. In some embodiments, the cell is a cell expressing a Pdcd1 gene, an Adora2a gene, and a Ctla4 gene, which function as immune repressors.

In some embodiments, the Cas is Cas9 or its variant. In some embodiments, the Cas is selected from a wild-type Cas9, a Cas9 nickase (Cas9n), dCas9, Cpf1, Sniper-Cas9, LZ3 Cas9, HiFi Cas9, eSpCas9, SpCas9-HF1, HypaCas9, xCas9, evoCas9, and SuperFi-Cas9.

In some embodiments, the Cas is in a form of a fusion protein with an effector protein, e.g., another enzyme. For example, a Cas, e.g., dCas9, can be fused with a Krüppel-associated box (Krab) effector domain.

In some embodiments, the Cas is pre-complexed with the first, second, and third gRNAs, e.g., in a form of an RNP.

In some embodiments, the method for disrupting PAC optionally comprises: introducing into the cell a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the method for disrupting PAC optionally comprises: introducing into the cell a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the cytoplasmic domain comprises a CD247 cytoplasmic domain.

In some embodiments, the one or more costimulatory signaling regions are selected from CD28, CD27, CD134 (OX40), and CD137 (4-1BB).

In some embodiments, the PAC disruption is introduced into a cell via a CRISPR method, while the CAR was introduced into cells via lentivirus-based genome integration. In some embodiments, one or more vectors encoding a Cas (e.g., Cas9), the first gRNA, second gRNA, third gRNA, and a CAR may be introduced into a cell.

In some embodiments, a nucleotide sequence encoding a Cas (e.g., Cas9), a nucleotide sequence encoding the first gRNA, a nucleotide sequence encoding the second gRNA, a nucleotide sequence encoding the third gRNA, and a nucleotide sequence encoding a CAR may be present in one vector or multiple vectors, such as in two, three, four, or five vectors.

### Methods of Treatment and Uses

In some embodiments, the present disclosure provides a method of treating a disease in a subject in need thereof, comprising administering to the subject an engineered immune cell comprising: a Cas, a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene, wherein the Pdcd1 gene, the Adora2a gene, and the Ctla4 are disrupted.

In some embodiments, the disease is selected from cancers and tumors. In some embodiments, the cancer is selected from bladder cancer, breast cancer, cervical cancer, gynecologic cancers, head and neck cancers, kidney cancers, liver cancer, lung cancer, lymphoma, mesothelioma, myeloma, ovarian cancer, prostate cancer, skin cancer, thyroid cancer, uterine cancer, and vaginal and vulvar cancers. In some embodiments, the tumor is selected from benign tumors, premalignant tumors, and malignant tumors.

A Cas can be introduced into an immune cell in the form of a polynucleotide sequence (e.g., in a plasmid or as mRNA encoding the Cas protein) or in the form of RNP. The gRNAs can be introduced into an immune cell in the form of a polynucleotide sequence encoding the gRNA or in the form of RNP.

In some embodiments, a Cas, a first gRNA, a second gRNA, and a third gRNA may be introduced into an immune cell via one or more vectors expressing the Cas9 and gRNA as described herein. In some embodiments, a Cas, a first gRNA, a second gRNA, and a third gRNA can be delivered into immune cells in other forms, e.g., in the form of ribonucleoprotein (RNP), where the guide RNAs are mixed with a Cas (e.g., Cas 9) enzyme protein. RNPs can be delivered into cells via electroporation.

In some embodiments, the method of treating the disease comprises administering to the subject an engineered immune cell comprising one or more polynucleotides encoding the Cas and the first, second, and third gRNAs. In some embodiments, a nucleotide sequence encoding the Cas, a nucleotide sequence encoding the first gRNA, a nucleotide sequence encoding the second gRNA, and a nucleotide sequence encoding the third gRNA can be included in a single vector or in multiple vectors, such as 2, 3, or 4 vectors.

In some embodiments, the first, second, and third gRNAs are single-molecule guide RNAs (sgRNAs).

In some embodiments, the engineered immune cell is an engineered T cell.

In some embodiments, the Cas enzyme is Cas9 or its variant. In some embodiments, the Cas is selected from a wild-type Cas9, a Cas9 nickase (Cas9n), dCas9, Cpf1, Sniper-Cas9, LZ3 Cas9, HiFi Cas9, eSpCas9, SpCas9-HF1, HypaCas9, xCas9, evoCas9, and SuperFi-Cas9.

In some embodiments, the engineered immune cell engineered with PAC disruption optionally comprises a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the PAC disruption is introduced into an immune cell via a CRISPR method, while the CAR was introduced into the immune cell via lentivirus-based genome integration. In some embodiments, one or more vectors encoding a Cas (e.g., Cas 9), the first gRNA, second gRNA, third gRNA, and a CAR may be introduced into a cell.

In some embodiments, the immune cell engineered with PAC disruption optionally comprises a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the cytoplasmic domain comprises a CD247 cytoplasmic domain.

In some embodiments, the one or more costimulatory signaling regions are selected from CD28, CD27, CD134 (OX40), and CD137 (4-1BB).

In some embodiments, the engineered immune cell optionally further comprises additional gene knockout. For example, in allogeneic CAR-T (also known as the “off-the-shelf” CAR-T) cells, additional genes (e.g., TCR and CD52) are knocked out compared to the autologous CAR-T to eliminate the individual-specific immunogenicity. Such allogeneic CAR-T may be further engineered as disclosed herein to disrupt the PAC genes and used in a method described herein.

In some embodiments, a nucleotide sequence encoding a Cas (e.g., Cas 9), a nucleotide sequence encoding the first gRNA, a nucleotide sequence encoding the second gRNA, a nucleotide sequence encoding the third gRNA, and a nucleotide sequence encoding a CAR may be present in one vector or multiple vectors, e.g., two, three, four, or five vectors.

The therapeutically effective amount of the engineered immune cells, e.g., engineered T cells, can be determined based on factors of a particular subject, including size, age, sex, weight, and condition of the subject. Dosages can be ascertained and adjusted by those skilled in the art from this disclosure and the knowledge in the art.

In some embodiments, the engineered immune cells, e.g., engineered T cells, can be administered in any pharmaceutically acceptable vehicle. In some embodiments, a therapeutically effective amount of about 1×10⁵ to 1×10¹², 1×10⁶ to 1×10¹¹, 1×10⁶ to 1×10¹⁰, 1×10⁶ to 1×10⁹, 1×10⁷ to 1×10¹¹, 1×10⁷ to 1×10¹⁰, 1×10⁷ to 1×10⁹, or 1×10⁸ to 1×10⁹ cells are administered. In some embodiments, a dose of about 1×10⁶, 1×10⁷, 1×10⁸, 1×10⁹, 1×10¹⁰ or 1×10¹¹ cells are administered to a subject having cancer or tumor.

In some embodiments, the purity in populations comprising the engineered immune cells, e.g., engineered T cells, ranges from about 70% to about 75%, about 75% to about 80%, about 80% to about 85%, preferably about 85% to about 90%, about 90% to about 95%, and about 95% to about 100%.

The engineered immune cells, e.g., engineered T cells, can be administered by, for example, injection or catheter. The engineered immune cells, e.g., engineered T cells, may also be administered by minimally invasive surgical techniques.

### Nucleic Acids and Engineered Cells

In some embodiments, the present disclosure provides a vector comprising one or more of nucleotide sequences wherein the nucleotide sequences are selected from: (a) a first nucleotide sequence encoding a first gRNA comprising a spacer sequence complementary with a first target domain of a Pdcd1 gene; (b) a second nucleotide sequence encoding a second gRNA comprising a second spacer sequence complementary with a second target domain of an Adora2a gene; and (c) a third nucleotide sequence encoding a third gRNA comprising a third spacer sequence complementary with a third target domain of a Ctla4 gene; and (d) a fourth nucleotide sequence encoding a Cas (e.g., Cas 9).

In some embodiments, a nucleotide sequence encoding the Cas (e.g., Cas 9), a nucleotide sequence encoding the first gRNA, a nucleotide sequence encoding the second gRNA, and a nucleotide sequence encoding the third gRNA can be present in a single vector or in multiple vectors, such as two, three, or four vectors.

In some embodiments, the vector is selected from an AAV vector, an adenovirus vector, a vaccinia virus vector, retrovirus, and a herpes simplex virus vector. In some embodiments, the vector is or comprises a lentiviral vector.

In some embodiments, the present disclosure provides an engineered cell comprising: a Cas (e.g., Cas 9), a first guide RNA (gRNA) comprising a first spacer sequence targeting a Pdcd1 gene, a second gRNA comprising a second spacer sequence targeting an Adora2a gene, and a third gRNA comprising a third spacer sequence targeting a Ctla4 gene.

In some embodiments, the present disclosure provides an engineered cell comprising: one or more polynucleotides encoding a Cas (e.g., Cas 9), a first gRNA comprising a first spacer sequence targeting a Pdcd1 gene, a second gRNA comprising a second spacer sequence targeting an Adora2a gene, and a third gRNA comprising a third spacer sequence targeting a Ctla4 gene. As described herein, the polynucleotides the Cas (e.g., Cas 9), the first, second, and third gRNAs may be delivered in one, two, three, or four vectors.

In some embodiments, the engineered cell optionally further comprises a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the engineered cell optionally further comprises a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

In some embodiments, the vector further comprises a nucleotide sequence encoding a CAR as well as a promoter, such as U6.

In some embodiments, the present disclosure provides a nucleic acid encoding a chimeric antigen receptor (CAR), wherein the CAR comprises an antigen binding domain, a transmembrane domain, one or more costimulatory signaling region, and a cytoplasmic domain comprising an immunoreceptor tyrosine-based activation motif (ITAM), wherein the antigen binding domain is capable of binding a cancer antigen.

In some embodiments, the cytoplasmic domain comprises a CD247 cytoplasmic domain.

In some embodiments, the one or more costimulatory signaling regions are selected from CD28, CD27, CD134 (OX40), and CD137 (4-1BB).

In some embodiments, a chimeric antigen receptor further comprises one or more extracellular leader domains and/or one or more extracellular hinge domains. In some embodiments, one or more extracellular hinge domains comprise a CD28 extracellular hinge domain, CD8a extracellular hinge domain, or an IgG4 extracellular hinge domain.

In some embodiments, a transmembrane domain comprises a CD28, CD8a, CD64, CD32a, CD32c, CD16a, TRLI, TLR2, TLR3, TRL4, TLR5, TLR6, TLR7, TLRS, or TLR9 transmembrane domain.

In some embodiments, the antigen binding domain is a scFv that is capable of binding a cancer antigen.

In some embodiments, a nucleotide sequence encoding a Cas (e.g., Cas 9), a nucleotide sequence encoding the first gRNA, a nucleotide sequence encoding the second gRNA, a nucleotide sequence encoding the third gRNA, and a nucleotide sequence encoding a CAR may be in delivered in one vector or multiple vectors, e.g., two, three, four, or five vectors.

In some embodiments, the engineered cell optionally further comprises additional gene knockout. In some embodiments, in an engineered cell disclosed herein, additional genes (e.g., TCR and CD52) may optionally further be knocked out. For example, in allogeneic CAR-T (also known as the “off-the-shelf” CAR-T) cells, additional genes (e.g., TCR and CD52) are knocked out compared to the autologous CAR-T to eliminate the individual-specific immunogenicity. Such allogeneic CAR-T may be further engineered as disclosed herein to disrupt the PAC genes.

In some embodiments, a cell is engineered with a PAC disruption using a CRISPR method, e.g., delivery of RNPs via electroporation, and the cell is engineered with a CAR via lentivirus-based genome integration.

In some embodiments, the immune cells are selected from a macrophage, monocyte, dendritic cell, T cells, B cells and/or NK cells. In some embodiments, the immune cells are T cells. In some embodiments, the T cells are selected from CD4+ cells, CD8+ cells, Gamma delta T cells (γδT cells), NK T cells and/or regulatory T cells (Treg). In some embodiments, the immune cells are TIL (Tumor infiltrated lymphocyte). For example, TILs can be isolated from tumors, expanded, genetically engineered as disclosed herein, and then injected back to patients.

In some embodiments, immune cells are harvested from a human subject, e.g., a donor subject who is a heathy human, or a subject in need of treatment, and then genetically engineered as described herein. For example, when iPSC is used to make an immune cell such as T cell or NK cell, the donor subject can be a healthy human.

In some embodiments, T cells are harvested from a human subject having cancer or tumor and then genetically engineered as described herein. The engineered T cells are then administrated to the subject for treatment of cancer or tumor.

For example, white blood cells from a subject diagnosed with a cancer or tumor are harvested, T cells are isolated and cultured, and the T cells are engineered as described herein, e.g., to comprise a PAC triple knock out and comprise a CAR. The engineered T cells prepared for administration to a subject can comprise a purified population of cells, for example CD4+ T cells. Those having ordinary skill in the art can readily determine the percentage of genetically modified immune cells in a population using various well-known methods, such as fluorescence activated cell sorting (FACS).

### Guide RNAs

In a CRISPR/Cas system, a guide RNA (gRNA) is a synthetic RNA composed of a scaffold sequence necessary for Cas-binding and a user-defined nucleotide spacer that defines the gene target to be modified. The strand of genomic DNA that is bound by the spacer is typically referred to as the complementary strand or a target domain of a gene. And the other strand of DNA is typically referred to as the non-complementary strand.

Any suitable stretch of a nucleotide sequence on a Pdcd1 gene, an Adora2a gene, or Ctla4 gene can be used as a target domain for designing the respective gRNAs. For example, target domains on the respective genes may be selected close to the 5′ end of the CDS, avoiding secondary structures. The GC content of an gRNA is preferably within 40-60%. One can use a gRNA designing tool, which provides scores indicating gRNA editing efficiency and/or gRNA off-targeting specificity for selection of appropriate gRNAs.

In some embodiments, the present disclosure provides a first guide RNA (gRNA) comprising a first spacer sequence targeting a Pdcd1 gene. Any stretch of a nucleotide sequence on a Pdcd1 gene can be used as a target domain for designing a first guide RNA. In some embodiments, the first guide RNA comprises a sequence as set forth in SEQ ID NO: 1: CAGCTTGTCCAACTGGTCGG

In some embodiments, the present disclosure provides a second gRNA comprising a second spacer sequence targeting an Adora2a gene. Any stretch of a nucleotide sequence on an Adora2a gene can be used as a target domain for designing a first guide RNA. In some embodiments, the second guide RNA comprises a sequence as set forth in SEQ ID NO: 2: AGCACACAAGCACGTTACCC

In some embodiments, the present disclosure provides a third gRNA comprising a third spacer sequence targeting a Ctla4 gene. Any stretch of a nucleotide sequence on a Ctla4 gene can be used as a target domain for designing a first guide RNA. In some embodiments, the third guide RNA comprises a sequence as set forth in SEQ ID NO: 3:GGACTGAGAGCTGTTGACAC

### CRISPR-Cas Proteins

In various embodiments, the present disclosure involves a Cas protein or a variant or a mutant of any of the variants thereof. For example, all variants and mutants of Cas proteins, such as those described in Koonin et al., Curr Opin Microbiol. 2017 June; 37:67-78, can be used in a method, composition, or kit disclosed herein. For example, Cas9 proteins, either wild type or genetically modified, can be used in a method, composition, or kit disclosed herein. The Cas enzymes used in Base Editing and Prime Editing can also be used here.

For instance, Cas9 has many variants. By introducing mutations into amino acid sequences, those variants have different characteristics in terms of their specificity and efficiency. The Cas9 protein that can be used herein can be selected from SpCas9 (Cas9 isolated from Streptococcus pyogenes), SaCas9 (Cas9 isolated from Staphylococcus aureus), StCas9 (Cas9 isolated from Streptococcus thermophilus), NmCas9 (Cas9 isolated from Neisseria meningitidis), FnCas9 (Cas9 isolated from Francisella novicida), CjCas9 (Cas9 isolated from Campylobacter jejuni), ScCas9 (Cas9 isolated from Streptococcus canis), Sniper-Cas9, LZ3 Cas9, HiFi Cas9, eSpCas9 (Slaymaker, et al., Science, Vol. 351, Issue 6268, 84-88, 2016), SpCas9-HF1 (Kleinstiver, et al., Nature, 529, 490-495, 2016), HypaCas9, xCas9, evoCas9, SuperFi-Cas9 (Bravo et al., Nature, 603 (7900): 343-347), and any variants or mutant forms of these Cas proteins listed above. This list is only to provide some exemplary options and is not exclusive.

In various embodiments, a Cas protein can be fused with an effector protein, e.g., another enzyme. For example, a Cas, e.g., dCas9, can be fused with a Krüppel-associated box (Krab) effector domain, as used in a CRISPRi system. CRISPRi downregulates the expression of a target gene by repressive gene regulation, instead of disrupting the expression of a target gene by editing a genomic sequence directly. In the CRISPRi system, a Cas9 mutant, dCas9, is fused with another effector protein (e.g., Krab). The dCas9/gRNA guides the Krab effector protein to a specific location in the genome, and then the Krab effector protein downregulates the expression of the targeting gene by interacting with cis-regulatory elements. Such fusions of a Cas and an effector protein can be used in the methods, compositions, and kits disclosed herein.

### Pharmaceutical Compositions

In some embodiments, the disclosure provides pharmaceutical compositions comprising a population of engineered cells, e.g., engineered immune cells, e.g., engineered T cells, described herein. In some embodiments, a pharmaceutical composition comprises a pharmaceutically acceptable carrier.

In some embodiments, pharmaceutical compositions comprising engineered immune cells, e.g., engineered T cells, are provided as sterile liquid preparations, e.g., isotonic aqueous solutions, suspensions, emulsions, dispersions, or viscous compositions, which may be buffered to a selected pH. In some embodiments, pharmaceutically acceptable carrier is selected from water, saline, phosphate buffered saline, polyol (for example, glycerol, propylene glycol, liquid polyethylene glycol, and the like) and suitable mixtures thereof.

In some embodiments, the disclosure provides pharmaceutical compositions comprising a nucleic acid or a vector described herein. In some embodiments, a pharmaceutical composition comprises a pharmaceutically acceptable carrier.

### Kits

In some embodiments, the present disclosure provides a kit comprising a Cas (e.g., Cas9), a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene.

In some embodiments, the present disclosure provides a kit comprising a Cas (e.g., Cas9) pre-complexed with one or more (e.g., 2 or 3) of a first gRNA comprising a first spacer sequence targeting the Pdcd1 gene, a second gRNA comprising a second spacer sequence targeting the Adora2a gene, and a third gRNA comprising a third spacer sequence targeting the Ctla4 gene. In some embodiments, the Cas (e.g., Cas9) and the one or more of the gRNAs are precomplexed to form an RNP (ribonucleoprotein).

In some embodiments, the present disclosure provides a kit comprising one or more vectors comprising one or more (e.g., e.g., two, three, four, or five) nucleotide sequences encoding a Cas (e.g., Cas9), a first guide RNA (gRNA) comprising a first spacer sequence targeting the Pdcd1 gene, a second gRNA comprising a second spacer sequence targeting the Adora2a gene, a third gRNA comprising a third spacer sequence targeting the Ctla4 gene, and a CAR.

### EXAMPLES

The following examples are provided to describe the disclosure in greater detail. They are intended to illustrate, not to limit, the disclosure.

### Example 1: In Vivo Screen for Combinatorial Checkpoint Blockades to Boost T Cells

To identify potential candidates for a combined immunotherapy, we applied a 4-gRNA multiplexed library in an in vivo screen for boosted tumor-infiltrating T cells (TILs). Following a multiplexed CRISPR library construction strategy, as further detailed below, we genetically engineered CD8+ T cells collected from OT-I mice. To investigate synergistic or additive anti-tumor efficacies of multiple gene knockouts, we engineered the T cells with a library of four gRNAs simultaneously.

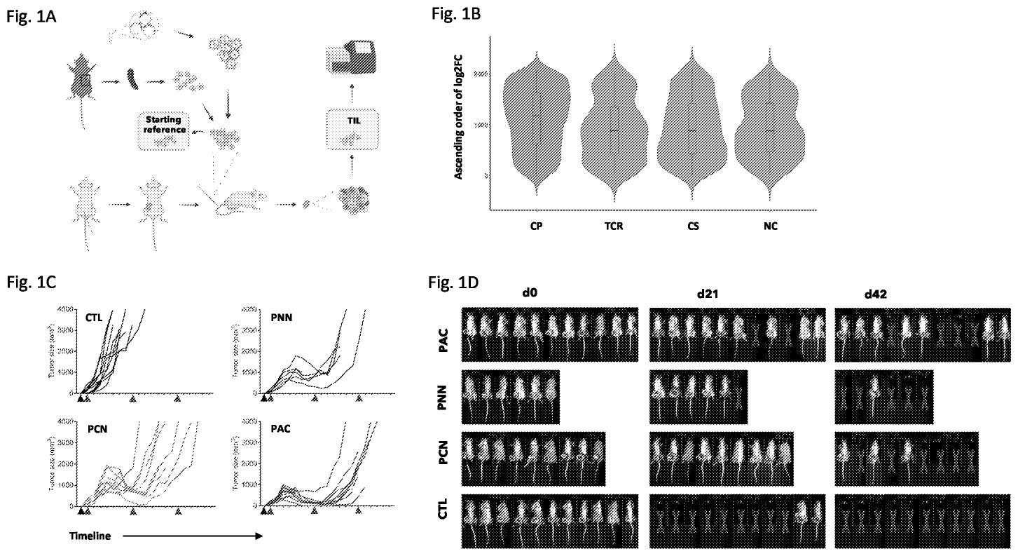

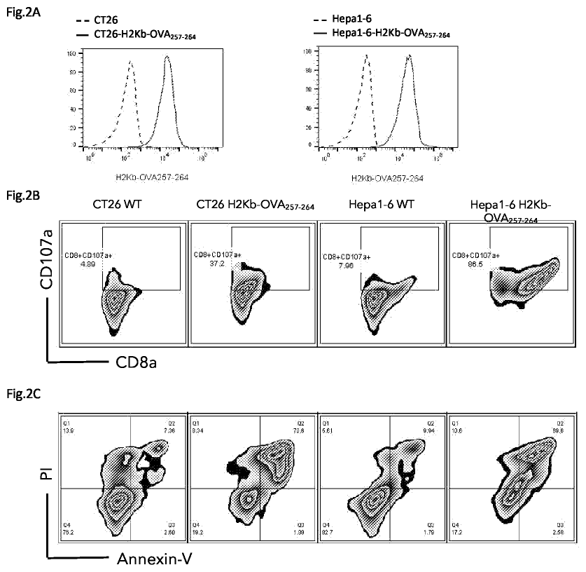

The engineered T cells were screened for activation capability in a tumor environment. The engineered T cells were injected into recipient mice inoculated with Hepa1-6 cells with stable H2Kb-OVA₂₅₇₋₂₆₄ expression (FIG. 1A and FIG. 2). At the endpoint of the screening, the engineered T cells that were present in the tumors in the mice were isolated by Fluorescence-activated Cell Sorting (FACS) and subjected to NGS (next generation sequencing) characterization.

More specifically, the in vivo screening library was designed to target six checkpoint genes (Btla, Pdcd1, Tigit, Ctla4, Havcr2 and Adora2a) and included all fifty-six possible combinations, composed of fifteen 4-gRNA combinations, twenty 3-gRNA combinations, fifteen 2-gRNA combinations and six single-gRNA combinations (denoted as “CP group” herein). Moreover, for each combination, we used non-targeting control gRNAs to fill the unoccupied positions if the number of the targeting gRNAs is less than 4. For example, for the six single-gRNA combinations, each of them contains three non-targeting control gRNAs to fill the unoccupied positions. For comparison, we also included combinations targeting two other groups of genes: one included four genes (Lat, Zap70, Cd3e, and CD247) involved in the first signaling of T cell activation (fifteen combinations, denoted as “TCR group” herein), the other included five co-stimulatory molecules (Il2ra, Tnfrsf9, Tnfrsf4, Tnfrsf18 and CD28) involved in the secondary signaling of T cell (thirty combinations, denoted as “CS group” herein). T cells engineered by combinations from the TCR group and CS group should be incapable of T cell activation. All together, we included 101 distinct combinations targeting one to four genes of the CP group, the TCR group, and the CS group. For each distinct combination, we designed a group of six gRNA-combos in the library to eliminate biases of individual guide RNA. Another eighty-four combinations included only non-targeting control gRNAs, which served as negative controls (denoted as “NT group” herein). The sequences of the gRNA combinations are listed in SEQ ID NOs: 14-703. The screening was conducted in three independent batches.

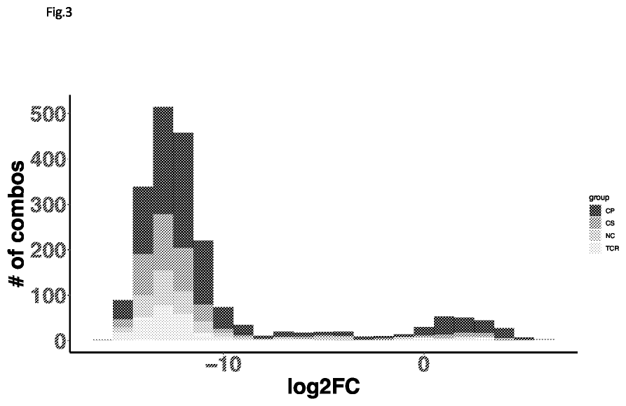

We calculated log2 transformed fold-change (log2FC) values to show the relative abundance of each gRNA combination in the tumor infiltrated lymphocytes (TIL) relative to the engineered T cells before being injected into the recipient mice (“SR,” representing “starting reference”) (further described below). It was contemplated that the T cells enriched in the tumors gained functions relevant to anti-tumor immunity, which were reflected by the gRNA combinations with high log2FC values. As shown in FIG. 3, most T cells did not successfully get enriched in the tumors and showed negative log2FC values (FIG. 3). Among the four groups (CP, TCR, CS, and NC), more T cells from the CP group showed a positive log2FC value (FIG. 1B, FIG. 3). These results indicated the effectiveness of the screening model.

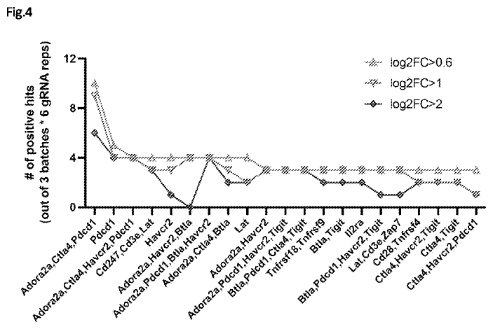

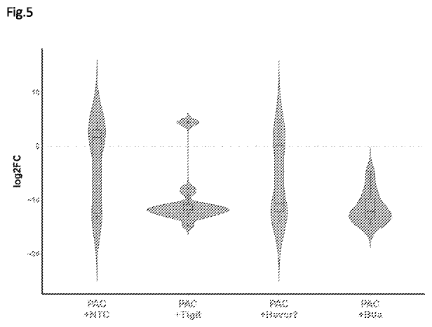

We ranked all gRNA combinations based on the corresponding T cell enrichments in the tumors from three screening batches and identified a top candidate of 3-gRNA combination that simultaneously targets Pdcd1 , Adora2a and Ctla4 (denoted as “PAC” herein) (FIG. 4). Among all the gRNA combinations, the PAC combination exhibited the best reproducibility across three independent batches of screen and different groups of gRNAs. We also examined other 4gRNA-combinations that included gRNAs targeting these three genes and found that only this specific combination maximumly activated the infiltrated T cells in tumors (FIG. 5). These results indicate the importance of identifying the precise combination of targets, as the anti-tumor ability of T cells may not be positively strengthened by knocking out more checkpoint genes.

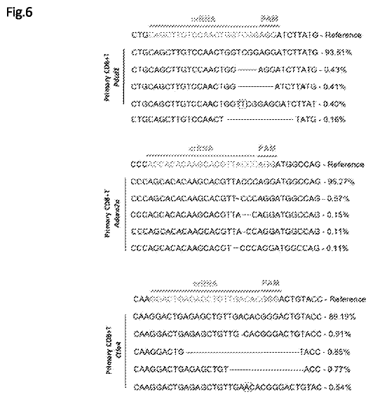

Next, we performed validation experiments to confirm the screen results. We prepared T cells knocked out only at the Pdcd1 loci (denoted as “PNN”), at Pdcd1 and Ctla4 loci (denoted as “PCN”), as well as Pdcd1 , Ctla4, and Adora2a loci (“PAC”). The knockout efficiencies of the gRNAs were confirmed (FIG. 6). We also shuffled gRNAs when making combos to eliminate biases from individual gRNA. The gRNAs used in the validation experiment were randomly picked from the gRNAs used in the screening experiment. For example, there were six different gRNA combinations targeting the PAC in the screening library. In the validation experiment, an individual gRNA was picked randomly from those gRNAs that have been used in the screening library. Thus, the specific gRNA combination to target PAC in the validation experiments were not present in the screening library.

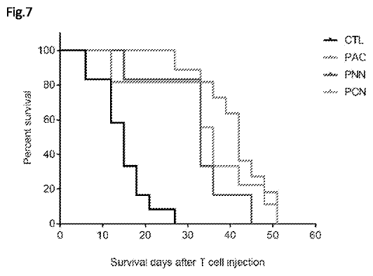

The engineered T cells were injected intravenously into the recipient mice inoculated with Hepa1-6 cancer cells expressing H2Kb-OVA₂₅₇₋₂₆₄. After the T cell therapy, the weight loss of the mice and the tumor size was monitored for eight weeks. We found that the growth of tumor size of the other two groups (PNN and PCN) was all controlled at different levels. T cells engineered by the PAC combination showed the best anti-tumor immune responses compared to T cells engineered by PCN or PNN, which were reflected by the tumor size and the survival rate of the mice (FIG. 1C-1D & FIG. 7). The rates of tumor growth in the PCN and PNN groups were slower compared to the CTRL group, in which non-engineered T cells were used.

These results indicated that the multiplexed CRISPR screen is an effective way to look for candidates for potential combinatorial immune checkpoint blockades, and for other potential combinatoral pathway blockades.

### Example 2: Screen Library and Vector Design

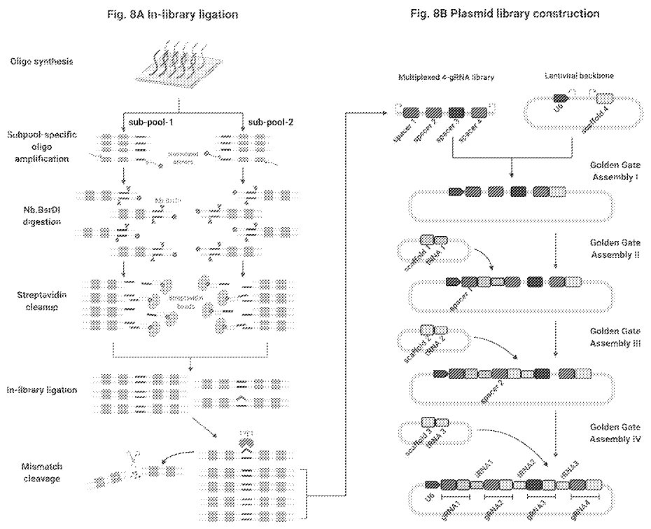

A screen library was designed and constructed via in-library ligation and plasmid library construction (FIG. 8A and FIG. 8B).

As shown in FIG. 8A, the in-library ligation was performed as follows. An oligo library with two sub-pools was synthesized. Each sub-pool was PCR amplified by specific PCR handles located at the 5′ and 3′ endsof oligos in two separate reactions. The reverse PCR primer of sub-pool-1 and the forward PCR primer of the sub pool-2 were modified with 5′-biotin. The oligos of SEQ ID NO: 4 and SEQ ID NO: 5 were used as a pair of primers to amplify the sub-pool-1 (Table 1). The oligos of SEQ ID NO: 6 and SEQ ID NO: 7 were used as a pair of primers to amplify the sub-pool-2 (Table 1). These PCR products had two Nb.BsrDI recognition sites around the complementary region, one on the top strand and the other on the bottom strand. After the Nb.BsrDI digestion, staggered nicks were generated, which released a small fragment from the 3′-end of sub-pool-1 PCR products and the 5′-end of sub-pool-2 PCR products. These small fragments were then captured and removed by streptavidin beads through the biotin modifications on them. The two 3′-over hanged sub-pools were then pooled together and ligated through sequence complementation. Each product from the sub-pool-1 had one and only one counterpart in the sub-pool-2, and ligation products carrying mismatches were digested by T7E1.

As shown in FIG. 8B, the plasmid library construction was performed as follows. The correct ligation products from the in-library ligation were cloned into a lentiviral backbone by golden gate assembly between the human U6 promoter and the Scaffold-4. Three more sequential golden gate assembly were performed to clone the following elements: the Scaffold-1 and human Gln-tRNA, the Scaffold-2 and human Gly-tRNA, and the Scaffold-3 and human Pro-tRNA. The final plasmid comprises a U6-driven multiplexed gRNA cassette separated by three tRNAs.

As noted above, for the check point blockade screening library, we included a group of six immune checkpoint genes (CP group), a group of four genes involved in the first signaling of T cell activation (TCR group) and a group of five co-stimulatory molecules involved in the secondary signaling (CS group). Within each group, all possible 4 gRNA combinations, 3 gRNA combinations, 2 gRNA combinations, and single gRNA construct were designed. For each construct, the unoccupied position was placed with non-targeting control gRNA if the number of the targeting gRNAs is less than four. Further, all combinations were represented by six groups of gRNAs that are distinct from each other. Additionally, 84 combinations containing only the non-targeting control gRNAs (NT group) were included and served as negative control. This screen library composed a total of 101 gene combinations represented by 606 gRNA groups and 84 negative control combinations including on non-targeting gRNAs.

For the screening part, a multiplexed CRISPR knockout vector that contained a 4-sgRNA tandem cassette (as illustrated in FIG. 8B) and a mKate2 reporter was generated. Using this vector, up to 4 checkpoints (e.g., shown as gRNA1, gRNA2, gRNA3, and gRNA4 illustrated in FIG. 8B) could be disrupted in single cell. A mKate2 reporter was used in order to separate the engineered T cells that infiltrated into the tumors.

For the validation part, a multiplexed CRISPR knockout vector that contained a Pdcd1-Adora2a-Ctla4 gRNA tandem cassette and a mKate2 reporter was generated (SEQ ID NO: 704). A vector that contained a Pdcd1-NTC-NTC gRNA tandem cassette and a BFP reporter were created as control, in which one NTC gRNAs replaced the Adora2a gRNA, one NTC gRNA replaced the Ctla4 gRNA, and a BFP reporter replaced the mKate2 reporter. A vector that contained a Pdcd1-Ctla4-NTC sgRNA tandem cassette and a BFP reporter was created as a control, in which one NTC gRNAs replaced the Adora2a gRNA and a BFP reporter replaced the mKate2 reporter.

TABLE 1

| | |
|---|---|
| Spacer of Pdcd1 | SEQ ID NO: 1: CAGCTTGTCCAACTGGTCGG |
| Spacer of Adora2a | SEQ ID NO: 2: AGCACACAAGCACGTTACCC |
| Spacer of Ctla4 | SEQ ID NO: 3: GGACTGAGAGCTGTTGACAC |
| F-BsrDI-1 | SEQ ID NO: 4: GACCGCGTCTCACACCG |
| R-BsrDI-1-biotin | SEQ ID NO: 5: CTGCGCTCCACGAGCCCGACGCAATG |
| F-BsrDI-2-biotin | SEQ ID NO: 6: CTGGCGTGGTCGCGTGCTCGGCAATG |
| F-BsrDI-2 | SEQ ID NO: 7: GATCAGGGCCGTCTCGAAAC |
| Murine nest-F | SEQ ID NO: 8: GGACTATCATATGCTTACCG |
| Murine nest-R | SEQ ID NO: 9: GCCCAGAattcTCGCATTC |
| Fwd-Libseq-G12 | SEQ ID NO: 10: |
| | AATGATACGGCGACCACCGAgatctACACTATAGCCTACAC |
| | TCTTTCCCTACACGACGCTCTTCCGATCTTGTGGAAAGGAC |
| | GAAACAC |
| Rev-Libseq-G12 | SEQ ID NO: 11: |
| | CAAGCAGAAGACGGCATACGAGATGGAGATGAGTGACTGGA |
| | GTTCAGACGTGTGCTCTTCCGATCTTGCTAGGACCGGCCTT |
| | AAAG |
| Fwd-Libseq-G23 | SEQ ID NO: 12: |
| | AATGATACGGCGACCACCGAGATCTACACTCTTTCCCTACA |
| | CGACGCTCTTCCGATCTGCTGGTTCCATGGTGTA |
| Rev-Libseq-G23 | SEQ ID NO: 13: |
| | CAAGCAGAAGACGGCATACGAGATCGAGTAATGTGACTGGA |
| | GTTCAGACGTGTGCTCTTCCGATCTCCTTAGCCGCTAATAG |
| | GTGAGC |

### Example 3: Screen and Validation Experiments

### Tumor Cells

Hepa1-6 cells were transduced with H-2Kb-OVA₂₅₇₋₂₆₄-expressing lentivirus. And the H-2Kb-OVA_{257.-264} expression in a mono-clone was validated via flow cytometry. The resulted cell line was named as Hepa1-6-H-2Kb-OVA_{257.264}. The established Hepa1-6-H-2K^{b}-OVA₂₅₇₋₂₆₄ cells were further transduced with a lentiviral vector (lenti-EF-1a-luciferase-T2A-BSD) for luciferase stable expression.

### Mice Models

Primary T cells were isolated from OT-I or Cas9+OT-I mice, which were bred from OT-I and Cas9 mouse obtained from the Jackson Laboratory. The tumor was inoculated to the NOD-Prkdc^{scid} Il2rg^{null}/Shjh mice purchased from Shanghai Jihui Laboratory Animal Care. The T cell donor mice were 10˜12 weeks old. The tumor recipient mice were 6˜8 weeks old. All mice were housed in standard individually ventilated and pathogen-free conditions in the laboratory facility of the Westlake University, under that animal protocol (AP #21-016-MLJ). All mice were used in accordance with Institutional Animal Care and Use Committee (IACUC) guidelines for Westlake University.

### T Cell Isolation and Culture

Spleens were isolated from the Cas9+OT-I mice, followed by mashing through 40 μm filter and RBCs lysis (BD Pharm Lyse). CD8+ T cells were purified by negative selection via CD8a+ T cell isolation Kit (Milteny). Cells were stimulated with 100U/ml recombinant human IL-2 (Peprotech), 1 μg/ml anti-mouse CD3ε(Ultraleaf, Clone 145-2C11, Biolegend) and 0.5 μg/ml anti-mouse CD28 (Ultraleaf, Clone 37.51, Biolegend) and cultured in RPMI-1640 with 10% FBS, 10 mM HEPES (Gibco), 100 μM non-essential amino acids (Gibco), 1 mM Sodium Pyruvate (Gibco), 50 μM β-mercaptoethanol (Sigma), 50 U/ml penicillin, and 50 μg/ml streptomycin (Gibco).

### T Cell Transduction, Transduction Efficiency Test, and Gene Editing Efficiency Test

After ex vivo stimulation for 24 h, CD8+ T cells were transduced with lentivirus in the presence of polybrene at 8 μg/ml during spinfection at 2,000 g for 2 h at 32° C. At 48 h after transduction, T cells were collected for transduction efficiency test via flow cytometry and adoptive transfer.

In a validation experiment, CD8+ T cells were transduced with lentivirus for 2 times at 24 h and 48 h after isolation. At 24 h after second transduction, T cells were collected for transduction efficiency test via flow cytometry and adoptive transfer after sorting via FACS. The gene editing efficiency was tested in T cells with a Pdcd1 -Adora2a-C′tla4 combined disruption. At 48 h after second transduction, mKate2+ T cells were sorted via FACS and pelleted for gDNA extraction. Then, the sgRNA target sequences of each gene were amplified by 2-step PCR for NGS sequencing. The list of oligos used in gene editing efficiency test were included in Table 1.

### Antigen Specificity Test for OT-I T Cells

OT-I CD8+ T cells were co-cultured with either Hepa1-6 cells or Hepa1-6 expressing H-2Kb-OVA₂₅₇₋₂₆₄ cells for 2 h and 48 h. In the 2 h test, cells were co-cultured at the presence of anti-CD107 (Biolegend). After 2 h, all cells were collected and stained with anti-CD8a (Biolegend) for degranulation analysis via flow cytometry (Cytoflex, Beckman). After 48 h, all cells were collected and stained with anti-CD8a, PI and Annexin V (Biolegend) for target cell apoptosis analysis via flow cytometry (Cytoflex, Beckman). All FCM Data were analyzed by Flowjo.

### Screening Experiment

Hepa1-6 cells expressing H-2K^{b}-OVA₂₅₇₋₂₆₄ were mixed with matrigel (1:1 volume) and injected subcutaneously into the right flank of NPSG mice at 1×10⁶/recipient. At d12 after tumor cell inoculation, 1×10⁷ CD8+ T cells with screening library transduction (5%˜10% mKate2+ cells in total cells) were adoptively transferred into each recipient via i.v. injection. Meanwhile, 2˜3×10⁶ CD8+ T cells with screening library transduction were frozen as a starting reference (SR). Weight loss and tumor size was measured at d0 and d7 after T cell injection. On d7 after injection, the tumor was collected and cut into small fragments. After consecutively mashing through 100 μm and 40 μm filters, RBCs in the cell suspension were lysed. Then, the tumor infiltrating CD8+ T cells were enriched by density gradient centrifugation via Lymphprep (StenCell). Cells at the interface were carefully collected and washed by PBS. Then, the cells were re-suspended into PBS and stained with anti-mouse CD8a for 30 mins on ice. Finally, CD8+mKate2+ TILs were sorted via FACS (BD Fusion). A total of 20,000-40,000 CD8+mKate2+ TIL could be collected per tumor. TIL from 3-4 recipient mice were mixed together and pelleted with carrier cells (Raji cell) at 1:50 (CD8+ T cells: carrier cells) for genomic DNA extraction.

### Genomic DNA Extraction and sgRNA Library PCR Amplification

Genomic DNA extraction was performed using TIANamp Genomic DNA kit (TIANGEN) and finally resuspended in 50 μl nuclease free water. To prepare the gRNA NGS library for the SR sample, all gDNA were amplified on thermocycling with parameters of 98° C. for 30 sec, 20˜22 cycles of (98° C. for 10 sec, 64° C. for 30 sec, 72° C. for 20 sec), 72° C. for 2 min. One NGS library generated amplicons covering the 1st and the 2nd gRNAs (G12 library), and another NGS library generated amplicons covering the 2nd and the 3rd gRNAs (G23 library). Primers of SEQ ID NO: 10 and SEQ ID NO: 11 were used as a pair of primers to amplify the G12 library. Primers of SEQ ID NO: 12 and SEQ ID NO: 13 were used as a pair of primers to amplify the G23 library. To prepare the gRNA NGS library for the TIL sample, two-step amplification was applied. In the 1st step, PCR reaction (400˜800 ng DNA input per reaction, 2˜4 reactions per sample) was performed using Ultra II Q5 Master Mix (NEB) with thermocycling parameters as 98° C. for 30 sec, 28˜30 cycles of (98° C. for 10 sec, 60° C. for 30 sec, 72° C. for 20 sec), 72° C. for 2 min. Primers of SEQ ID NO: 8 was used as the forward primer and SEQ ID NO: 9 was used as the reverse primer. And the PCR condition and primers of the 2nd step follows the condition of the SR library preparation, but with 8˜10 cycles.

The list of primers used in gene editing efficiency test were included in Table 1.

### Validation of Candidates

Hepa1-6 cells expressing H-2Kb-OVA257-264 with luciferase were mixed with matrigel (1:1 volume) and injected subcutaneously into the right flank of NPSG mice at 1×106/recipient. On d11-d12 after tumor cell inoculation, 1×10⁶ mKate2+ or BFP+ CD8+ T cells were sorted via FACS and adoptively transferred into each recipient via intravenous injection. Weight loss and tumor size were measured every 3 days after T cell injection. Meanwhile, the biological signal of tumor was monitored weekly by in vivo imaging via PHOTON IMAGERTM OPTIMA, in which luciferin was administered intraperitoneally 5 minutes prior to signal collection.

### Example 4: Data Analysis

In order to find the effective 4-gRNA combinations that enhance the capacity of the CD8+ T cell-mediated tumor elimination in vivo, the normalized read counts of each combination were used to compare their representatives between the TIL and SR libraries. Normalizations were conducted according to the depth of sequencing libraries. We calculated both the fold-change and the p-value for each 4-gRNA combination. The TIL and SR libraries were treated as two samples, and G12 library and G23 library of each sample were treated as technical replicates. We used the log2 fold-change of G12 and G23 between the TIL and SR libraries to pick out combinations for validations, which can be explained as Log2((Mean of TIL three batches g12+1)/(Mean of SR three batches g12+1)) and Log2((Mean of TIL three batches g23+1)/(Mean of SR three batches g23+1)).

Further embodiments are illustrated below.

Embodiment 1. A method for disrupting a Pdcd1 gene, an Adora2a gene, and an Ctla4 gene in a cell, comprising: introducing into a cell a Cas, a first guide RNA (gRNA) comprising a first spacer sequence targeting the Pdcd1 gene, a second gRNA comprising a second spacer sequence targeting the Adora2a gene, and a third gRNA comprising a third spacer sequence targeting the Ctla4 gene, wherein the Pdcd1 gene, the Adora2a gene, and the Ctla4 are disrupted.

Embodiment 2. The method of Embodiment 1, wherein the method comprises introducing into the cell one or more polynucleotides encoding the Cas and the first, second, and third gRNAs.

Embodiment 3. The method of Embodiment 1, wherein the Cas is pre-complexed with one or more of the first, second, and third gRNAs.

Embodiment 4. The method of any one of Embodiments 1 and 3, wherein the Cas pre-complexed with one or more of the first, second, and third gRNAs are introduced into the cell via electroporation.

Embodiment 5. The method of any one of Embodiments 1-4, wherein the first, second, and third gRNAs are single-molecule guide RNAs (sgRNAs).

Embodiment 6. The method of any one of Embodiments 1-5, wherein the first, second, and third gRNAs comprises SEQ ID NOs: 1, 2, and 3, respectively.

Embodiment 7. The method of any one of Embodiments 1-6, wherein the cell is an immune cell.

Embodiment 8. The method of any one of Embodiments 1-7, wherein the cell is a lymphocyte cell.

Embodiment 9. The method of any one of Embodiments 1-7, wherein the cell is a T cell.

Embodiment 10. The method of any one of Embodiments 1-9, wherein the Cas is a wild-type Cas9 or variant thereof.

Embodiment 11. The method of any one of Embodiments 1-10, further comprising: introducing into the cell a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 12. The method of any one of Embodiments 1-11, further comprising: introducing into the cell a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 13. The method of any one of Embodiments 11-12, wherein the cytoplasmic domain comprises a CD247 cytoplasmic domain.

Embodiment 14. The method of any one of Embodiments 11-13, wherein the one or more costimulatory signaling regions are selected from CD28, CD27, CD134 (OX40), and CD137 (4-1BB).

Embodiment 15. A method of treating cancer or tumor in a subject in need thereof, comprising administering to the subject an engineered immune cell comprising: a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, wherein the Pdcd1 gene, the Adora2a gene, and the Ctla4 are disrupted.

Embodiment 16. The method of Embodiment 15, wherein the method comprises administering to the subject an engineered immune cell comprising one or more polynucleotides encoding the Cas and the first, second, and third gRNAs.

Embodiment 17. The method of any one of Embodiments 15-16, wherein the first, second, and third gRNAs are single-molecule guide RNAs (sgRNAs).

Embodiment 18. The method of any one of Embodiments 15-17, wherein the first, second, and third gRNAs comprises SEQ ID NOs: 1, 2, and 3, respectively.

Embodiment 19. The method of any one of Embodiments 15-18, wherein the engineered immune cell is an engineered lymphocyte.

Embodiment 20. The method of any one of Embodiments 15-19, wherein the engineered immune cell is an engineered T cell.

Embodiment 21. The method of any one of Embodiments 15-20, wherein the Cas is a wild-type Cas9 or variant thereof.

Embodiment 22. The method of any one of Embodiments 15-21, wherein the engineered immune cell comprises a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 23. The method of any one of Embodiments 15-21, wherein the engineered immune cell comprises a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 24. The method of any one of Embodiments 22-23, wherein the cytoplasmic domain comprises a CD247 cytoplasmic domain.

Embodiment 25. The method of any one of Embodiments 22-24, wherein the one or more costimulatory signaling regions are selected from CD28, CD27, CD134 (OX40), and CD137 (4-1BB).

Embodiment 26. A vector comprising one or more of nucleotide sequences wherein the nucleotide sequences are selected from:
(a) a first nucleotide sequence encoding a first gRNA comprising a spacer sequence complementary with a first target domain of the Pdcd1 gene;
(b) a second nucleotide sequence encoding a second gRNA comprising a second spacer sequence complementary with a second target domain of the Adora2a gene;
(c) a third nucleotide sequence encoding a third gRNA comprising a third spacer sequence complementary with a third target domain of the Ctla4 gene; and
(c) a fourth nucleotide sequence encoding a Cas.

Embodiment 27. The vector of Embodiment 26, further comprising
a fifth nucleotide sequence encoding a CAR and p1 a promoter.

Embodiment 28. The vector of any one of Embodiments 26-27, wherein the vector is selected from an AAV vector, an adenovirus vector, retrovirus, a vaccinia virus vector, a herpes simplex virus vector, and a lentiviral vector.

Embodiment 29. A cell comprising: a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting an Ctla4 gene.

Embodiment 30. A cell comprising: one or more polynucleotides encoding a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene.

Embodiment 31. The cell of any one of Embodiments 29-30, further comprising a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 32. The cell of any one of Embodiments 29-30, further comprising: a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

Embodiment 33. The cell of any one of Embodiments 29-32, wherein the cell is a T cell from a human subject.

Embodiment 34. The cell of any one of Embodiments 29-32, wherein the cell is a T cell from a human subject having cancer or tumor.

Embodiment 35. A pharmaceutical composition comprising a population of the cell of Embodiment 33 or the T cell of Embodiment 34.

Embodiment 36. A kit comprising a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene.

Embodiment 37. A kit comprising one or more vectors comprising one or more nucleotide sequences encoding a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, and a CAR.

Embodiment 38. An engineered immune cell for treating cancer or tumor in a subject in need thereof, wherein the engineered immune cell comprises a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, wherein the Pdcd1 gene, the Adora2a gene, and the Ctla4 are disrupted.

Embodiment 39. Use of an engineered immune cell for the manufacture of a medicament for treating cancer or tumor, wherein the engineered immune cell comprises a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, wherein the Pdcd1 gene, the Adora2a gene, and the Ctla4 are disrupted.

While the disclosure has been particularly shown and described with reference to specific embodiments, it should be understood by those having skill in the art that various changes in form and detail may be made therein without departing from the spirit and scope of the present disclosure as disclosed herein.

1. A method for disrupting a Pdcd1 gene, an Adora2a gene, and an Ctla4 gene in a cell, comprising: introducing into a cell a Cas, a first guide RNA (gRNA) comprising a first spacer sequence targeting the Pdcd1 gene, a second gRNA comprising a second spacer sequence targeting the Adora2a gene, and a third gRNA comprising a third spacer sequence targeting the Ctla4 gene, wherein the Pdcd1 gene, the Adora2a gene, and the Ctla4 are disrupted.

2. The method of claim 1, wherein the method comprises introducing into the cell one or more polynucleotides encoding the Cas and the first, second, and third gRNAs.

3. The method of claim 1, wherein the Cas is pre-complexed with one or more of the first, second, and third gRNAs.

4. The method of claim 1, wherein the Cas pre-complexed with one or more of the first, second, and third gRNAs are introduced into the cell via electroporation.

5. The method of claim 1, wherein the first, second, and third gRNAs are single-molecule guide RNAs (sgRNAs).

6. The method of claim 1, wherein the first, second, and third gRNAs comprises SEQ ID NOs: 1, 2, and 3, respectively.

7. The method of claim 1, wherein the cell is an immune cell.

8. The method of claim 1, wherein the cell is a lymphocyte cell.

9. The method of claim 1, wherein the cell is a T cell.

10. The method of claim 1, wherein the Cas is a wild-type Cas9 or variant thereof.

11. The method of claim 1, further comprising: introducing into the cell a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

12. The method of claim 1, further comprising: introducing into the cell a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

13. The method of claim 11, wherein the cytoplasmic domain comprises a CD247 cytoplasmic domain.

14. The method of claim 11, wherein the one or more costimulatory signaling regions are selected from CD28, CD27, CD134 (OX40), and CD137 (4-1BB).

15. A method of treating cancer or tumor in a subject in need thereof, comprising administering to the subject an engineered immune cell comprising: a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, wherein the Pdcd1 gene, the Adora2a gene, and the Ctla4 are disrupted.

16. The method of claim 15, wherein the method comprises administering to the subject an engineered immune cell comprising one or more polynucleotides encoding the Cas and the first, second, and third gRNAs.

17. The method of claim 15, wherein the first, second, and third gRNAs are single-molecule guide RNAs (sgRNAs).

18. The method of claim 15, wherein the first, second, and third gRNAs comprises SEQ ID NOs: 1, 2, and 3, respectively.

19. The method of claim 15, wherein the engineered immune cell is an engineered lymphocyte.

20. The method of claim 15, wherein the engineered immune cell is an engineered T cell.

21. The method of claim 15, wherein the Cas is a wild-type Cas9 or variant thereof.

22. The method of claim 15, wherein the engineered immune cell comprises a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

23. The method of claim 15, wherein the engineered immune cell comprises a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

24. The method of claim 22, wherein the cytoplasmic domain comprises a CD247 cytoplasmic domain.

25. The method of claim 22, wherein the one or more costimulatory signaling regions are selected from CD28, CD27, CD134 (OX40), and CD137 (4-1BB).

26. A vector comprising one or more of nucleotide sequences wherein the nucleotide sequences are selected from:
(a) a first nucleotide sequence encoding a first gRNA comprising a spacer sequence complementary with a first target domain of the Pdcd1 gene;
(b) a second nucleotide sequence encoding a second gRNA comprising a second spacer sequence complementary with a second target domain of the Adora2a gene;
(c) a third nucleotide sequence encoding a third gRNA comprising a third spacer sequence complementary with a third target domain of the Ctla4 gene; and
(c) a fourth nucleotide sequence encoding a Cas.

27. The vector of claim 26, further comprising a fifth nucleotide sequence encoding a CAR and a promoter.

28. The vector of claim 26, wherein the vector is selected from an AAV vector, an adenovirus vector, retrovirus, a vaccinia virus vector, a herpes simplex virus vector, and a lentiviral vector.

29. A cell comprising: a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting an Ctla4 gene; or comprising: one or more polynucleotides encoding a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene.

30. (canceled)

31. The cell of claim 29, further comprising a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

32. The cell of claim 29, further comprising: a polynucleotide encoding a chimeric antigen receptor (CAR), wherein the CAR comprises an antigen binding domain, a transmembrane domain, one or more costimulatory domain, and a cytoplasmic domain comprising an immunoreceptor tyrosine-based activation motif (ITAM), wherein the antigen binding domain is capable of binding a cancer or tumor antigen.

33. The cell of claim 29, wherein the cell is a T cell from a human subject.

34. The cell of claim 29, wherein the cell is a T cell from a human subject having cancer or tumor.

35. A pharmaceutical composition comprising a population of the T cell of claim 3.

36. A kit comprising a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene; or comprising one or more vectors comprising one or more nucleotide sequences encoding a Cas, a first guide RNA (gRNA) comprising a first spacer sequence targeting a Pdcd1 gene, a second gRNA comprising a second spacer sequence targeting an Adora2a gene, a third gRNA comprising a third spacer sequence targeting a Ctla4 gene, and a CAR.

37.-39. (canceled)
